(19) United States
(12) Patent Application Publication
(10) Pub. No.: US 2015/0132337 A1
(43) Pub. Date: May 14, 2015

(54) COMBINATION GAS VACCINES AND THERAPEUTICS

(71) Applicant: NOVARTIS AG, BASEL (CH)

(72) Inventors: Guido GRANDI, Siena (IT); Immaculada MARGARIT Y ROS, Siena (IT); Sabrina CAPO, Siena (IT); Giuliano BENSI, Siena (IT); Andrea Guido Oreste MANETTI, Siena (IT)

(21) Appl. No.: 14/540,627

(22) Filed: Nov. 13, 2014

Related U.S. Application Data
(63) Continuation of application No. 12/561,619, filed on Sep. 17, 2009, now abandoned.
(60) Provisional application No. 61/097,551, filed on Sep. 17, 2008.

(51) Int. Cl. C07K 14/315

(52) U.S. Cl. CPC C07K 14/315
USPC 424/190.1; 435/252.33; 435/252.3; 435/252.31; 435/325; 435/254.2

(57) ABSTRACT

Compositions useful for reducing the risk of, preventing, and/or treating S. pyogenes (GAS) infections which comprise combinations of GAS antigens, nucleic acid molecules encoding the antigens, or antibodies which specifically bind to the antigens.

This application is a continuation of Ser. No. 12/561,619 filed on Sep. 17, 2009, which incorporates by reference and claims the benefit of Ser. No. 61/097,551 filed on Sep. 17, 2008.

This application incorporates by reference the contents of a 1.2 MB file created Sep. 16, 2009 and named “52611_US_NP_sequencelisting.txt,” which is the sequence listing for this application.

### FIELD OF THE INVENTION

The invention relates to the fields of immunology and vaccinology.

### BACKGROUND OF THE INVENTION

Group A streptococcus (“GAS,” S. pyogenes) is a frequent human pathogen, estimated to be present in between 5-15% of normal individuals without signs of disease. An acute infection occurs, however, when host defenses are compromised, when the organism is able to exert its virulence, or when the organism is introduced to vulnerable tissues or hosts. Related diseases include puerperal fever, scarlet fever, erysipelas, pharyngitis, impetigo, necrotizing fasciitis, myositis, and streptococcal toxic shock syndrome.

Efforts to develop a prophylactic vaccine for use against GAS have been ongoing for many decades. Currently, however, there are no GAS vaccines available to the public. There is a need in the art for such vaccines.

### BRIEF DESCRIPTION OF THE DRAWINGS

FIG. 1. Graph showing protective capacity of GAS antigen combinations in a subcutaneous challenge mouse model. “GAS57,” Spy0416; “GAS25,” Spy0167; “GAS40,” Spy0269 (SEQ ID NO:177); “dGAS57,” Spy0167 mutant D151A/S617A (SEQ ID NO:198); “dGAS25,” Spy0167 mutant P427L/W535F (SEQ ID NO:125).

FIG. 2. Photomicrograph of SDS-polyacrylamide gels demonstrating that Spy0416 point mutant D151A has lost the ability to cleave IL-8. “57,” Spy0416 (GAS57).

FIG. 3. Graph showing the results of an ELISA assay demonstrating that Spy0416 point mutant D151A has lost the ability to cleave IL-8.

FIG. 4A and FIG. 4B. Photomicrographs of SDS-polyacrylamide gels demonstrating that Spy0416 single mutants D151A and S617A and the mutant D151A+S617A have lost Spy0416 proteolytic activity.

FIG. 5. Graph showing the results of an ELISA assay demonstrating that single mutants D151A and S617A and Spy0416 mutant D151A+S617A have lost Spy0416 (“57”) proteolytic activity.

FIG. 6. Photomicrograph of an SDS-polyacrylamide gel demonstrating that wild-type Spy0416 is post-translationally modified into two polypeptide fragments of 150.5 kDa and 23.4 kDa.

FIG. 7. Photomicrograph of an SDS-polyacrylamide gel demonstrating that Spy0416 mutants D151A, S617A, and D151A+S617A are not post-translationally modified into two polypeptide fragments of 150.5 kDa and 23.4 kDa compared to wild-type (black arrows). A major band of 174 kDa corresponding to unprocessed protein is instead present in the lanes corresponding to inactive mutant strains (grey arrow). “57,” Spy0416.

FIGS. 8A-B. ELISA assay results demonstrating dose-dependent inhibition of Spy0416-mediated IL-8 cleavage by polyclonal antisera against Spy0416 (“57”) in two different experimental conditions. FIG. 8A, 8 hour incubation, 0.1 μg/ml of Spy0416. FIG. 8B, 24 hour incubation, 0.05 μg/ml of Spy0416.

FIG. 9. Graph showing results of hemolytic assay using E. coli extracts containing wild-type Spy0167 and Spy0167 mutant P427L.

FIG. 10. Photomicrograph of SDS-polyacrylamide gel showing purified Spy0167 mutant P427L.

FIG. 11. Graph showing results of hemolytic assay using purified wild-type Spy0167 and Spy0167 mutant P427L.

FIG. 12. Photomicrograph of SDS-polyacrylamide gel of E. coli lysate supernatants. Lane A, E. coli negative control; lane B, rSpy0167 wild-type, without tag; lane C, rSpy0167 P427L, without tag; and lane D, purified rSpy0167 wild-type, without tag (5 mg).

FIG. 13. Graph demonstrating that under the same conditions, Spy0167 mutant P427L is 1000 times less hemolytic than wild-type Spy0167.

FIG. 14. Graph demonstrating effects of cholesterol on hemolysis by wild-type Spy0167 and Spy0167 mutant P427L.

FIGS. 15A-B. Photomicrographs of SDS-PAGE analysis of total tag-less proteins in cell extracts. FIG. 15A, expression of Spy0167 wild-type and P427L tag-less proteins; FIG. 15B, expression of Spy0167 P427L+W535, P427L+C530G, and P427L+C530G+W535F tag-less proteins.

FIG. 16. Photomicrograph of SDS-PAGE analysis of total His-tagged proteins in cell extracts.

FIG. 17. Photomicrograph of SDS-PAGE analysis of purified His-tagged proteins.

FIGS. 18A-B. Photomicrographs of SDS-PAGE analysis of purified tag-less proteins. FIG. 18A, Lanes: A, Spy0167 wild-type tag-less; B, Spy0167 P427L tag-less; molecular weight markers (116-66.2-45-35-25-18.4-14.4); black arrow indicates Spy0167 protein purified from mutants and wild-type clones. FIG. 18B, lane A, Spy0167 Wild Type tag-less (3 μg), lane B, Spy0167 P427L-W535F tag-less (3 μg); molecular weight markers (116-66.2-45-35-25-18.4-14.4); black arrow indicates Spy0167 protein purified from mutants and wild-type clones.

FIG. 19. Photomicrograph of SDS-PAGE analysis of purified tag-less Spy0167 (“GAS25”) wild-type protein. Samples of different purification lots of wild-type Spy0167 were analyzed under reducing and non-reducing conditions.

FIG. 20. Graph showing results of hemolysis tests of His-tagged Spy0167 mutants.

FIG. 21. Graph showing inhibition of Spy0167-induced hemolytic activity by anti-Spy0167 antiserum.

FIG. 22. Graph showing titration of anti-Spy0167 antiserum inhibition of Spy0167 hemolysis.

FIG. 23. Graph showing Spy0167 hemolytic activity titration.

FIG. 24. Graph showing titration of hemolytic activity of wild-type Spy0167, chemically detoxified wild-type Spy0167, and Spy0167 mutants (P427L; P427L+W535F).

FIG. 25. Graph showing titration of hemolytic activity of wild-type Spy0167 and Spy0167 mutants (P427L; P427L+W535F).

FIG. 26. Graph showing titration of hemolytic activity of wild-type Spy0167 and chemically detoxified wild-type Spy0167.

FIG. 27. Graph showing dilution of antiserum against Spy0167 (“gas25”) mutant P427L+W535F required to obtain 50% reduction of Spy0167 hemolytic activity (50 ng/ml Spy0167).

FIG. 28. Graph showing dilution of antiserum against Spy0167 (“gas25”) mutant P427L+W535F required to obtain 50% reduction of Spy0167 hemolytic activity (100 ng/ml Spy0167).

FIG. 29. Titration curve showing that hemolysis inhibition assays were performed with toxin concentrations which allow 100% hemolysis.

FIG. 31A-GG. Alignments of Spy0416 (“gas57”) antigens from different strains/M types. The catalytic triad (D, H, S) is in bold black characters. FIG. 31A, amino acids 1-50 (amino acid numbers at the top of each of FIG. 10A-GG refers to the amino acid sequence of Spy0416M1_SF370, SEQ ID NO:1); FIG. 31B, amino acids 51-100; FIG. 31C, amino acids 101-150; FIG. 31D, amino acids 151-200; FIG. 31E, amino acids 201-250; FIG. 31F, amino acids 251-300; FIG. 31G, amino acids 301-350; FIG. 31H, amino acids 351-400, FIG. 31I, amino acids 401-450; FIG. 31J, amino acids 451-500; FIG. 31K, amino acids 501-550; FIG. 31L, amino acids 551-600; FIG. 31M, amino acids 601-650; FIG. 31N, amino acids 651-700; FIG. 31O, amino acids 701-750; FIG. 31P, amino acids 751-800; FIG. 31Q, amino acids 801-850; FIG. 31R, amino acids 851-900; FIG. 31S, amino acids 901-950; FIG. 31T, amino acids 951-1000; FIG. 31U, amino acids 1001-1050; FIG. 31V, amino acids 1051-1100; FIG. 31W, amino acids 1101-1150; FIG. 31X, amino acids 1151-1200; FIG. 31Y, amino acids 1201-1250; FIG. 31Z, amino acids 1251-1300; FIG. 31AA, amino acids 1301-1350; FIG. 31BB, amino acids 1351-1400; FIG. 31CC, amino acids 1401-1450; FIG. 31DD, amino acids 1451-1500; FIG. 31EE, amino acids 1501-1550; FIG. 31FF, amino acids 1551-1600; FIG. 31GG, amino acids 1601-1650. M1_SF370, SEQ ID NO:1; M1_{—}31075, SEQ ID NO:2; M1_{—}31237, SEQ ID NO:3; M1_{—}3348, SEQ ID NO:4; M2_{—}34585, SEQ ID NO:5; M3,1_{—}21398, SEQ ID NO:6; M44-61_{—}20839, SEQ ID NO:7; M6,31_{—}20022, SEQ ID NO:8; M11_{—}20648, SEQ ID NO:9; M23_{—}2071, SEQ ID NO:10; M18,3_{—}40128, SEQ ID NO:11; M4_{—}10092, SEQ ID NO:12; M4_{—}30968, SEQ ID NO:13; M6,31_{—}22692, SEQ ID NO:14; M68,5_{—}22814, SEQ ID NO:15; M68_{—}23623, SEQ ID NO:16; M2_{—}10064, SEQ ID NO:17; M2_{—}10065, SEQ ID NO:18; M77_{—}10251, SEQ ID NO:19; M77_{—}10527, SEQ ID NO:20; M77_{—}20696, SEQ ID NO:21; M89_{—}21915, SEQ ID NO:22; M89_{—}23717, SEQ ID NO:23; M94_{—}10134, SEQ ID NO:24; M28_{—}10164, SEQ ID NO:25; M28_{—}10218, SEQ ID NO:26; M29_{—}10266, SEQ ID NO:27; M28_{—}10299, SEQ ID NO:28; M28_{—}30176, SEQ ID NO:29; M28_{—}30574, SEQ ID NO:30; M6,9_{—}21802, SEQ ID NO:31; M75_{—}10012, SEQ ID NO:32; M75_{—}20671, SEQ ID NO:33; M75_{—}30603, SEQ ID NO:34; M75_{—}30207, SEQ ID NO:35; M22_{—}20641, SEQ ID NO:36; M22_{—}23465, SEQ ID NO:37; M3,1_{—}30610, SEQ ID NO:38; M3,1_{—}40603, SEQ ID NO:39; M3,28_{—}24214, SEQ ID NO:40; M3,34_{—}10307, SEQ ID NO:41; M4_{—}40427, SEQ ID NO:42; M3_{—}2721, SEQ ID NO:43; M12_{—}10296, SEQ ID NO:44; M12_{—}10035, SEQ ID NO:45; M12_{—}20069, SEQ ID NO:46; M12_{—}22432, SEQ ID NO:47; M4_{—}40499, SEQ ID NO:48; and M6,1_{—}21259, SEQ ID NO:49.

FIGS. 32A-R. Alignments of Spy0269 (“gas40”) antigens from different strains/M types. FIG. 32A, amino acids 1-50 (amino acid numbers at the top of each of FIG. 10A-GG refers to the amino acid sequence of Spy0269M1_SF370, SEQ ID NO:50); FIG. 32B, amino acids 51-100; FIG. 32C, amino acids 101-150; FIG. 32D, amino acids 151-200; FIG. 32E, amino acids 201-250; FIG. 32F, amino acids 251-300; FIG. 32G, amino acids 301-350; FIG. 32H, amino acids 351-400, FIG. 32I, amino acids 401-450; FIG. 32J, amino acids 451-500; FIG. 32K, amino acids 501-550; FIG. 32L, amino acids 551-600; FIG. 32M, amino acids 601-650; FIG. 32N, amino acids 651-700; FIG. 32O, amino acids 701-750; FIG. 32P, amino acids 751-800; FIG. 32Q, amino acids 801-850; FIG. 32R, amino acids 851-874. M1_SF370, SEQ ID NO:50; clinicalisolate_{—}40s88, SEQ ID NO:51; M11_{—}2727, SEQ ID NO:52; M22_{—}20641, SEQ ID NO:53; M22_{—}23465, SEQ ID NO:54; M22_{—}23621, SEQ ID NO:55; M3.1_{—}30610, SEQ ID NO:56; M3.1_{—}40603, SEQ ID NO:57; M3.34_{—}10307, SEQ ID NO:58; M3_MGAS315, SEQ ID NO:59; M4_{—}40427, SEQ ID NO:60; M3_{—}2721, SEQ ID NO:61; M3_{—}3040, SEQ ID NO:62; M3_{—}3135, SEQ ID NO:63; M12_{—}10035, SEQ ID NO:64; M12_{—}22432, SEQ ID NO:65; M4_{—}40499, SEQ ID NO:66; M78_{—}3789, SEQ ID NO:67; M89_{—}10070, SEQ ID NO:68; M89_{—}21915, SEQ ID NO:69; M89_{—}23717, SEQ ID NO:70; M89_{—}5476, SEQ ID NO:71; M23_DSM2071, SEQ ID NO:72; M4_{—}2722, SEQ ID NO:73; M4_{—}10092, SEQ ID NO:74; M4_{—}30968, SEQ ID NO:75; M4_{—}2634, SEQ ID NO:76; M28_{—}10164, SEQ ID NO:77; M28_{—}10218, SEQ ID NO:78; M28_{—}10266, SEQ ID NO:79; M28_{—}10299, SEQ ID NO:80; M28_{—}30176, SEQ ID NO:81; M28_{—}4436, SEQ ID NO:82; M8_{—}2725, SEQ ID NO:83; M44_{—}3776, SEQ ID NO:84; M6_{—}2724, SEQ ID NO:85; M6_{—}2894, SEQ ID NO:86; M6_{—}3650, SEQ ID NO:87; M6_{—}5529, SEQ ID NO:88; M5, SEQ ID NO:89; M77_{—}4959, SEQ ID NO:90; M2_{—}10064, SEQ ID NO:91; M2_{—}10065, SEQ ID NO:92; M75_{—}5531, SEQ ID NO:93; M50_{—}4538, SEQ ID NO:94; M62_{—}5455, SEQ ID NO:95; M44_{—}5481, SEQ ID NO:96; M5_{—}4883, SEQ ID NO:97; M9?_{—}2720, SEQ ID NO:98; M2_{—}2726, SEQ ID NO:99; M12_{—}20296, SEQ ID NO:100; M1_{—}2580, SEQ ID NO: 101; M1_{—}2913, SEQ ID NO:102; M1_{—}3280, SEQ ID NO:103; M1_{—}3348, SEQ ID NO:104; M78_{—}3789, SEQ ID NO:105; M?_{—}2719, SEQ ID NO:106.

FIG. 33A-C. Alignments of Spy0167 (“gas25”) antigens from different strains/M types. FIG. 33A, amino acids 1-150 (amino acid numbers at the top of each of FIG. 10A-GG refers to the amino acid sequence of Spy0167M1_SF370, SEQ ID NO:107); FIG. 33B, amino acids 151-300; FIG. 33C, amino acids 301-500. M12_{—}2096, SEQ ID NO:108; M12_{—}9429, SEQ ID NO:109; M1_{—}5005, SEQ ID NO:110; M1_{—}3348, SEQ ID NO:111; M2_{—}10270, SEQ ID NO:112; M28_{—}6180, SEQ ID NO:13; M6_{—}10394, SEQ ID NO:114; M18_{—}8232, SEQ ID NO:115; M5_Manfredo, SEQ ID NO:116; M3_{—}315, SEQ ID NO:117; M3_SSI, SEQ ID NO:119; M4_{—}10750, SEQ ID NO:119.

FIG. 34. Graph showing results of whole blood bactericidal assays demonstrating that anti-glycoconjugate (GC) antibodies mediate killing of S. pyogenes.

FIG. 35. Graph showing results of whole blood bactericidal assays demonstrating that the combination of anti-glycoconjugate (GC) antibodies and antibodies generated against GAS antigen combinations enhance killing of S. pyogenes. “Freund,” Freund's adjuvant; “M1,” S. pyogenes M1 protein; “COMBO,” Spy0416 mutant D151A/S617A, Spy0167 mutant P427L/W535F5, and wild-type Spy0269; “GC,” GAS polysaccharide antigen conjugated to CRM197.

FIGS. 36A-D. Graphs showing results of cellular toxicity assays comparing various antigens with positive (tumor necrosis factor α, TNF-α) and negative (NT, not treated) controls. FIG. 36A, Spy0269 (GAS40, SEQ ID NO:177); FIG. 36B, Spy0416 (GAS57) and GAS57 mutant D151A/S617A (GAS57 DM, SEQ ID NO:198); FIG. 36C, Spy0167 (GAS25) and GAS25 mutant P427L/W535F (GAS25 DM, SEQ ID NO:125); FIG. 36D, glycoconjugate (GC).

FIGS. 37A-D. Graphs demonstrating validation of ELISA assay. FIG. 37A, Spy0167 (GAS25); FIG. 37B, Spy0269 (GAS40); FIG. 37C, Spy0416 (GAS57); FIG. 37D, glycoconjugate (GC).

FIGS. 38A-F. Graphs showing results of ELISA assays testing doses of individual antigens. FIGS. 38A, 38D, Spy0167 (GAS25) (two experiments); FIGS. 38B, 38E, Spy0416 (GAS57) (two experiments); FIGS. 38C, F, Spy0269 (GAS40) (two experiments). “GMT,” geometric mean titers.

FIG. 39. Graph showing results of ELISA and challenge assays testing combinations of Spy0416 mutant D151A/S617A (GAS57), wild-type Spy0269 (GAS40), and dose ranges of Spy0167 mutant P427L/W535F (GAS25) in alum.

FIG. 40. Analysis of LogNormal model adopted as first approximation of mean survival time (MST; Mu) analysis, demonstrating that Mu decreases with decreasing doses of Spy0167 (GAS25).

FIGS. 41A-B. Bar graphs demonstrating that antibodies to Spy0419 and Spy0167 block toxic activity. FIG. 41A, Spy0419 (GAS57). FIG. 41B, Spy0167 (GAS25). The titer is defined as the dilution factor required to neutralize 50% of maximum hemolysis.

### DETAILED DESCRIPTION OF THE INVENTION

The invention provides multi-component compositions which are useful for treating, reducing the risk of, and/or preventing S. pyogenes infections. In some embodiments compositions of the invention are vaccine compositions which provide effective prophylaxis against pharyngitis caused by S. pyogenes infections in children.

Compositions of the invention are useful for preventing and/or treating S. pyogenes infections. Compositions of the invention comprise combinations of GAS antigens, combinations of nucleic acid molecules encoding the GAS antigens, or combinations of antibodies which specifically bind to the GAS antigens. Compositions of the invention comprise combinations of two or more GAS antigens, combinations of one or more nucleic acids molecules encoding the GAS antigens, or combinations of antibodies which specifically bind to the GAS antigens. The GAS antigens are GAS protein antigens. “GAS protein antigen”, unless otherwise defined encompasses a full-length GAS protein as well as fragments, fusions and mutants of the GAS protein as described below. Some compositions further comprise a Group A polysaccharide antigen as defined below. The invention also includes compositions comprising mixtures of combinations of GAS antigens, combinations of nucleic acid molecules encoding the GAS antigens, and combinations of antibodies which specifically bind to the GAS antigens.

Compositions of the invention preferably have one or more of the following properties:
confer statistically significant protection against one or more S. pyogenes strains (e.g., M1 3348, M12 EMS, M23_{—}2071, M6 S43);
elicit antibodies which mediate in vitro bacterial killing (opsonophagocytic killing);
elicit antibodies which inactivate streptolysin O hemolytic activity;
elicit antibodies which block Spy0416 protease activity; and/or
elicit antibodies which prevent cell adhesion and/or cell division.

As described in the Examples below, compositions of the invention provide protection against different mouse-adapted S. pyogenes strains in lethal challenge models; elicit functional antibodies which neutralize potent toxins expressed by a majority of S. pyogenes strains; and mediate bacterial killing in vitro.

Some compositions of the invention comprise a combination of three or more different GAS antigens selected from Spy0167 (also referred to as streptolysin O, Spy0167, or “GAS25”); Spy0269 (also referred to as “GAS40”); Spy0416 (also referred to as “GAS57”); Spy0714 (also referred to as “GAS67”); Spy1390 (also referred to as “GAS89”); Spy2000 (also referred to as “GAS100”); a mutant Spy0167 protein as defined below; and a mutant Spy0416 as defined below.

In other embodiments compositions of the invention comprise only two GAS antigens, although the compositions may comprise antigens from other organisms as well as other components, as described below. Some compositions comprise as the two GAS antigens Spy0167 and a second GAS antigen selected from the group consisting of Spy0269; Spy0416; a mutant Spy0167 protein as described below; a mutant Spy0416 protein as described below; Spy0714; Spy1390; and Spy2000.

Other compositions comprise as the two GAS antigens Spy0269 and a second GAS antigen selected from the group consisting of Spy0167, the mutant Spy0167 protein, the mutant Spy0416 protein, Spy0714, Spy1390, and Spy2000.

Some compositions comprise as the two GAS antigens Spy0416 and a second GAS antigen selected from the group consisting of Spy0167, the mutant Spy0167 protein, the mutant Spy0416 protein, Spy0714, Spy1390, and Spy2000.

Other compositions comprise as the two GAS antigens the mutant Spy0167 protein and a second GAS antigen selected from the group consisting of Spy0167, Spy0269, Spy0416, the mutant Spy0416 protein, Spy0714, Spy1390, and Spy2000.

Other compositions comprise as the two GAS antigens the mutant Spy0416 protein and a second GAS antigen selected from the group consisting of Spy0167, Spy0269, Spy0416, the mutant Spy0167 protein, Spy0714, Spy1390, and Spy2000.

Some compositions comprise as the two GAS antigens Spy0714 and a second GAS antigen selected from the group consisting of Spy0167, Spy0269, Spy0416, the mutant Spy0167 protein, the mutant Spy0416 protein, Spy1390, and Spy2000.

Some compositions comprise as the two GAS antigens Spy1390 and a second GAS antigen selected from the group consisting of Spy0167, Spy0269, Spy0416, the mutant Spy0167 protein, the mutant Spy0416 protein, Spy0714, and Spy2000.

Other compositions comprise as the two GAS antigens Spy2000 and a second GAS antigen selected from the group consisting of Spy0167, Spy0269, Spy0416, the mutant Spy0167 protein, the mutant Spy0416 protein, Spy0714, and Spy1390.

Other GAS antigens which can be included in compositions of the invention include Spy0019 (GAS5), Spy0163 (GAS23), Spy0385 (GAS56), Spy0714 (GAS67), Spy0737 (GAS68), Spy1274 (GAS84), Spy1361 (GAS88), Spy1390 (GAS89), Spy1733 (GAS95), Spy1882 (GAS98), Spy1979 (GAS99), Spy2000 (GAS100), Spy2016 (GAS102), Spy0591 (GAS130), Spy1105 (GAS159), Spy1718 (GAS179), Spy2025 (GAS193), Spy2043 (GAS195), Spy1939 (GAS277), Spy1625 (GAS372), and Spy1134 (GAS561).

Preferred combinations of GAS antigens include the following, each of which may also include a GAS polysaccharide antigen, as described below, and/or an adjuvant, such as alum:
i. Spy0167 and Spy0269;
ii. Spy0167, Spy0269, and Spy0416;
iii. Spy0167 and Spy0416;
iv. Spy0167 mutant P427L/W535F and Spy0269;
v. Spy0167 mutant P427L/W535F, Spy0269, and Spy0416;
vi. Spy0167 mutant P427L/W535F and Spy0416;
vii. Spy0269 and Spy0416 mutant D151A/S617A;
viii. Spy0167, Spy0269, and Spy0416 mutant D151A/S617A;
ix. Spy0167 and Spy0416 mutant D151A/S617A;
x. Spy0167 mutant P427L/W535F, Spy0269, and Spy0416 mutant D151A/S617A; and
xi. Spy0167 mutant P427L/W535F and Spy0416 mutant D151A/S617A.

The compositions can contain other components, such as pharmaceutically acceptable vehicles, antigens of other microorganisms, adjuvants, etc. In particular, any of the compositions described herein may further comprise a Group A polysaccharide antigen as described below and/or an adjuvant, such as alum.

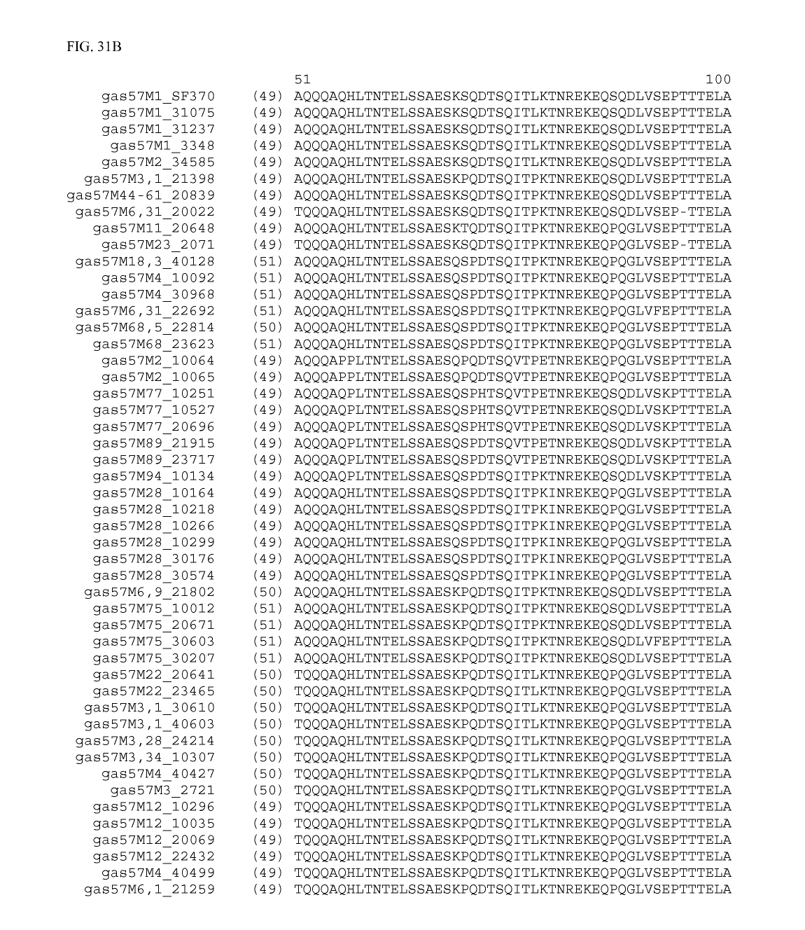

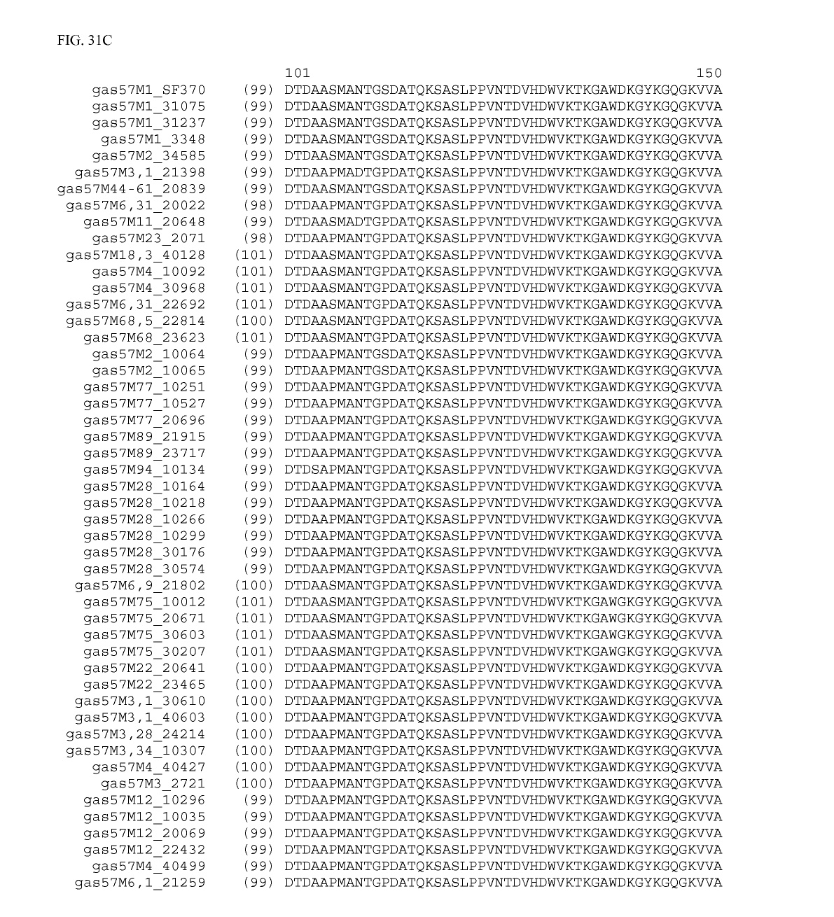

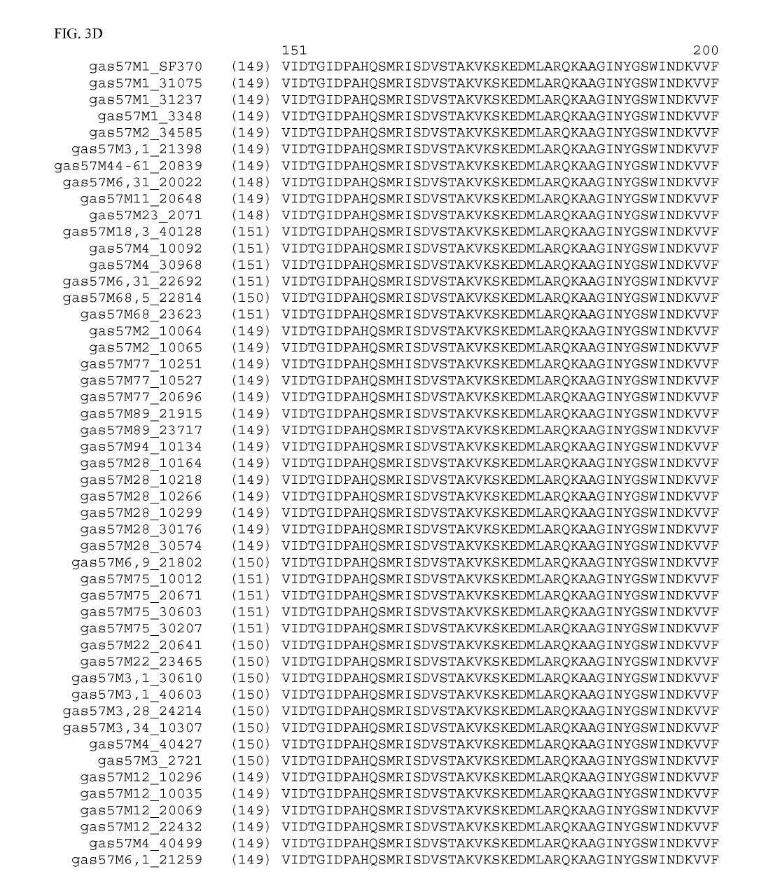

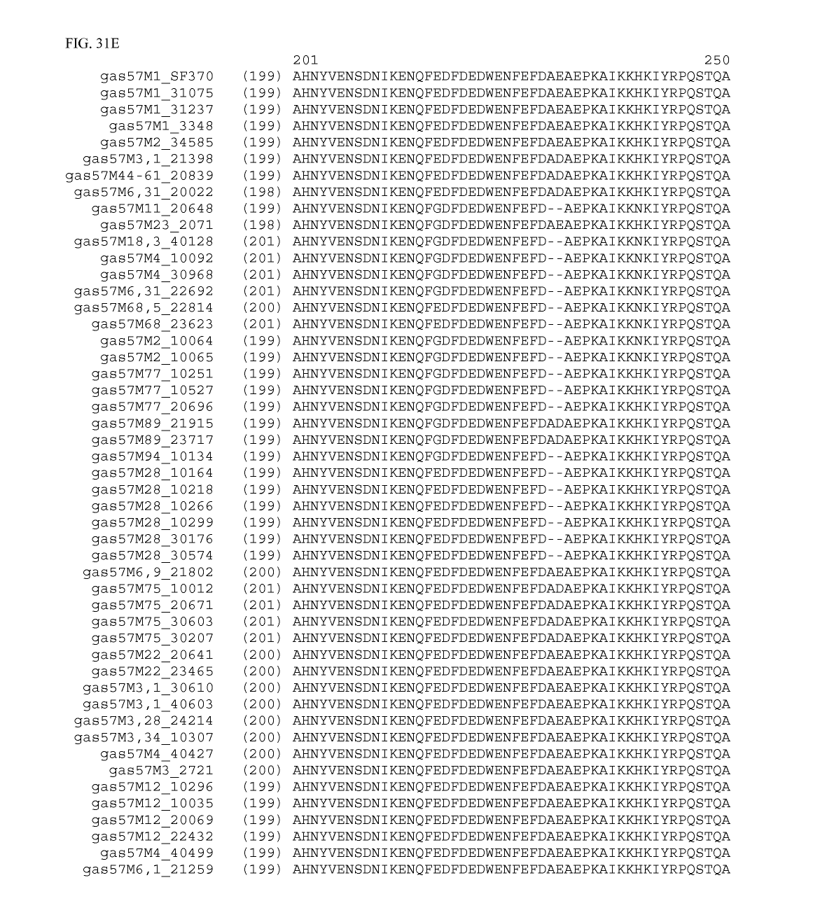

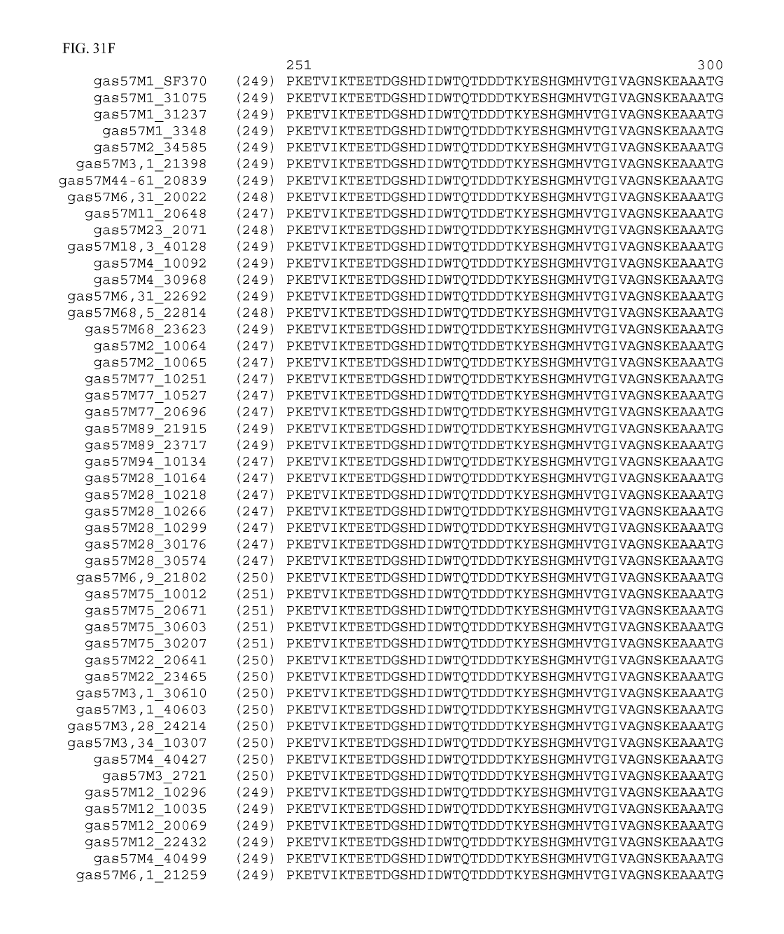

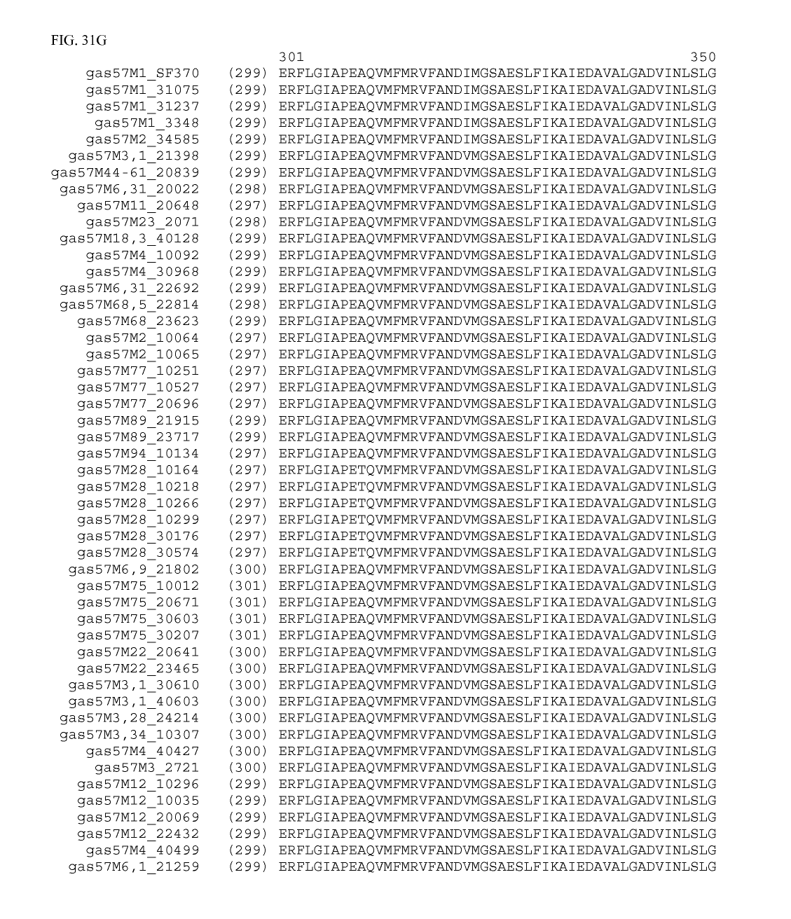

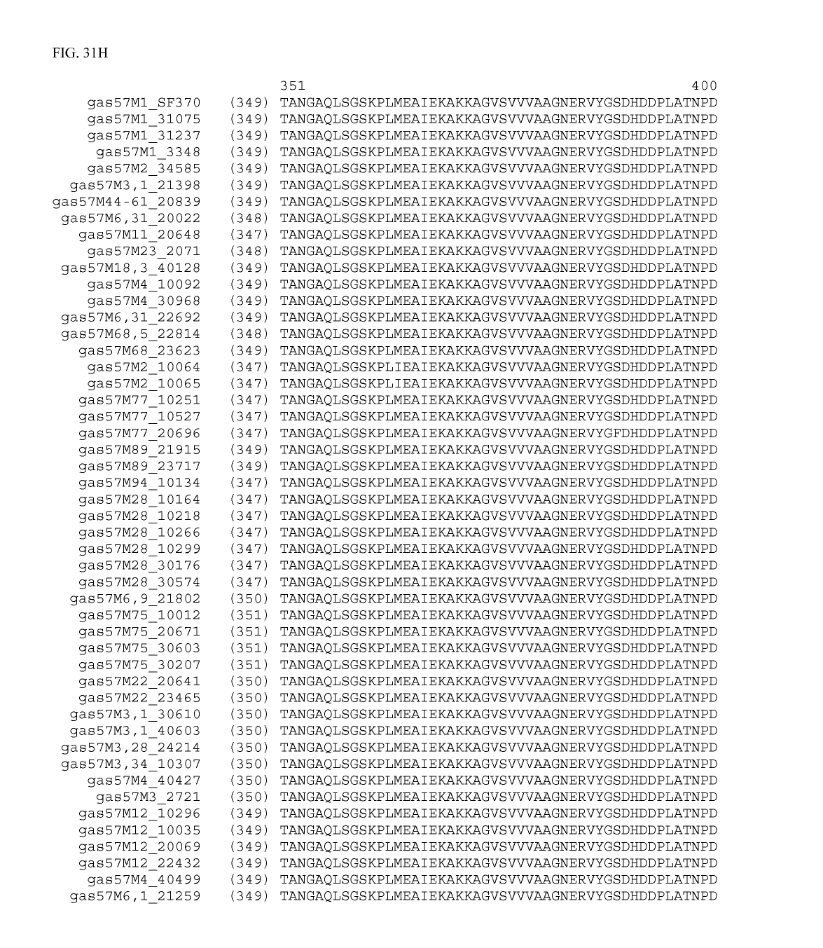

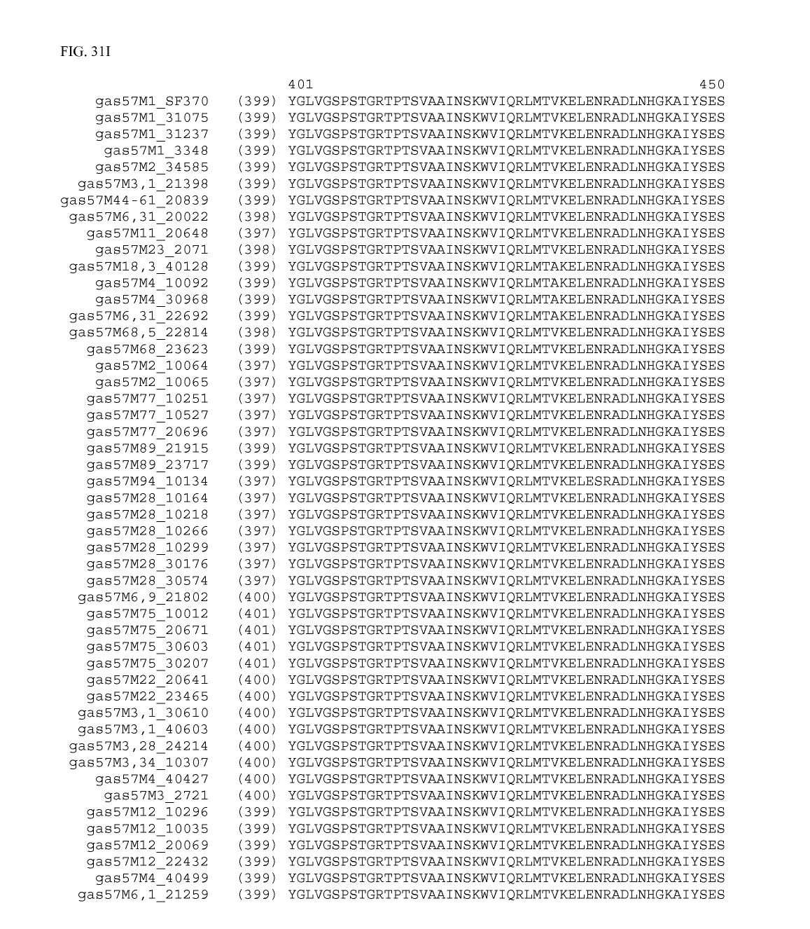

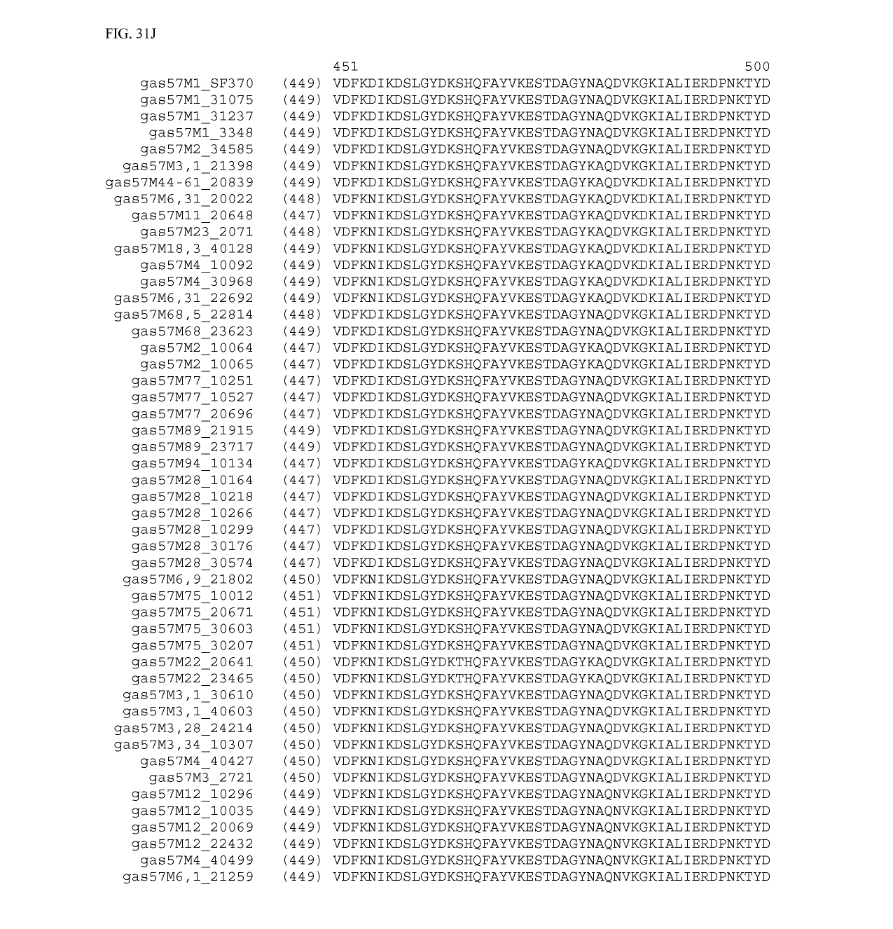

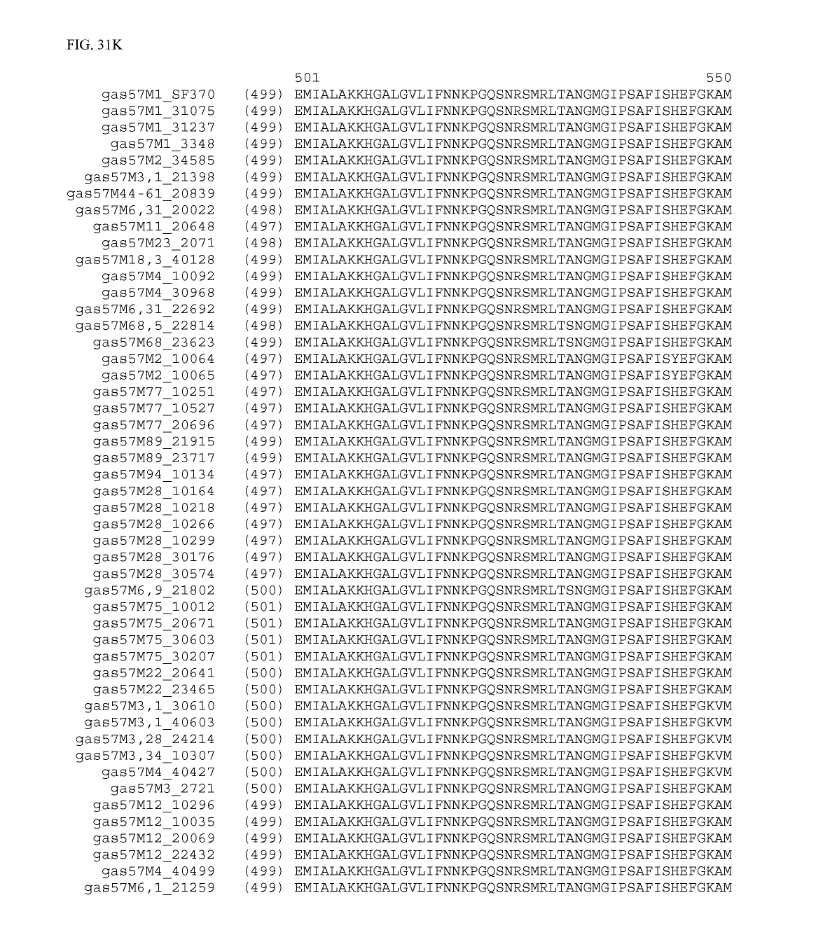

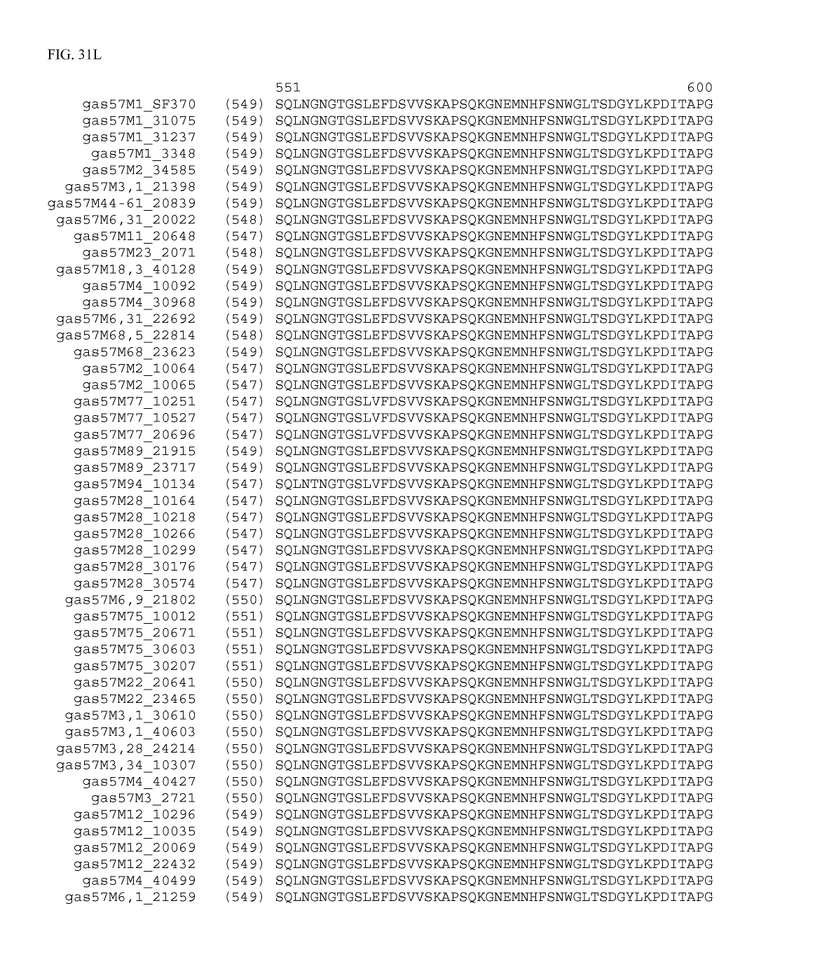

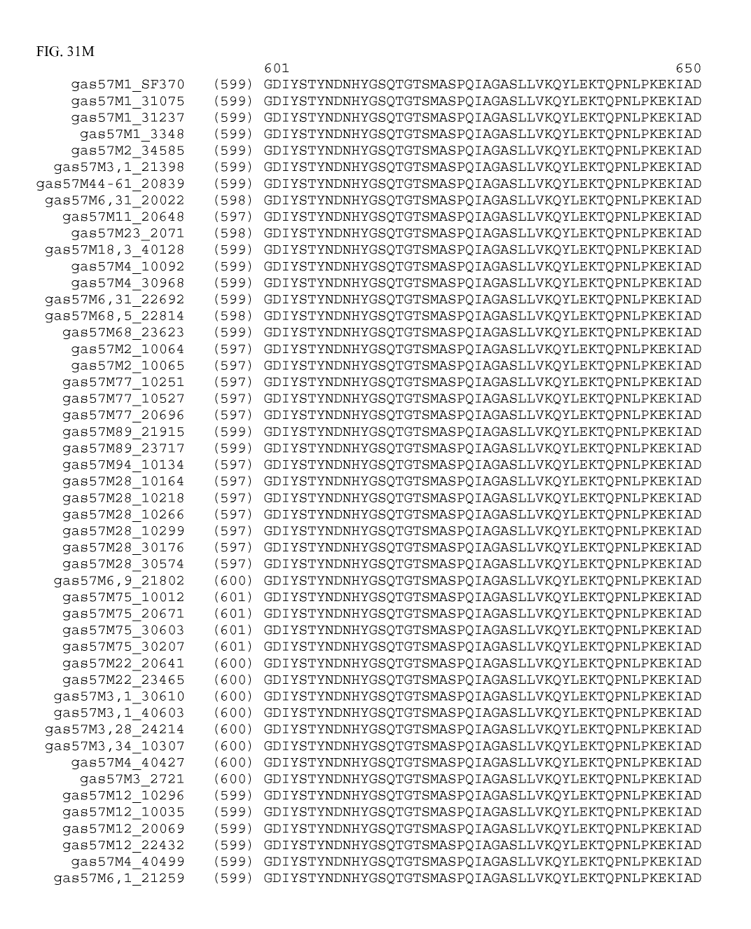

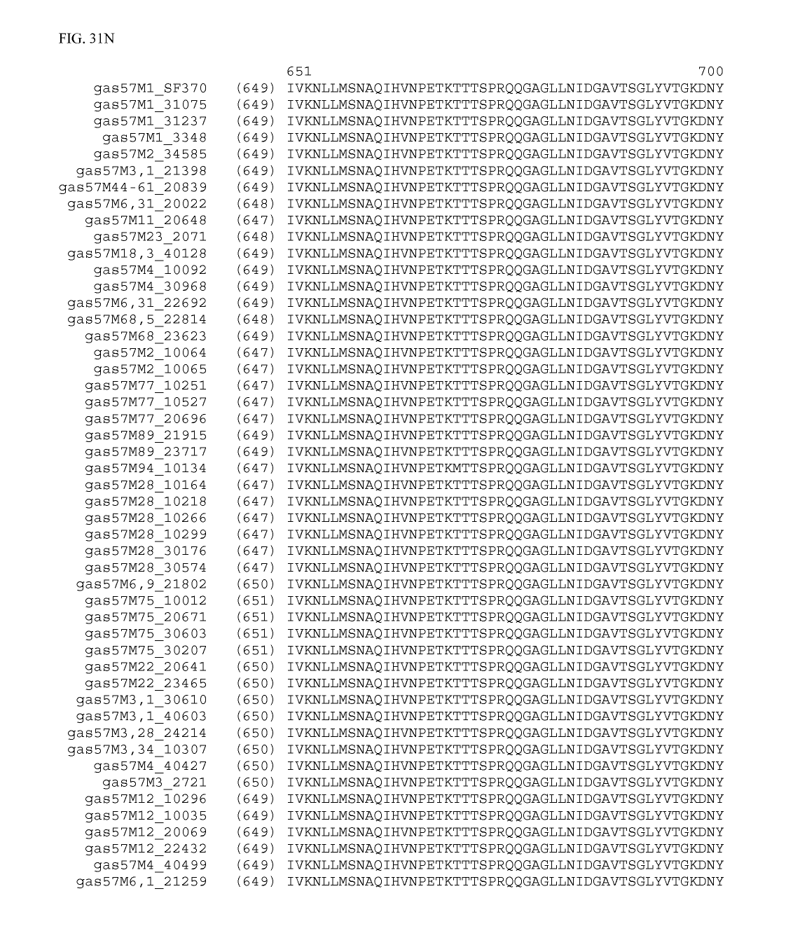

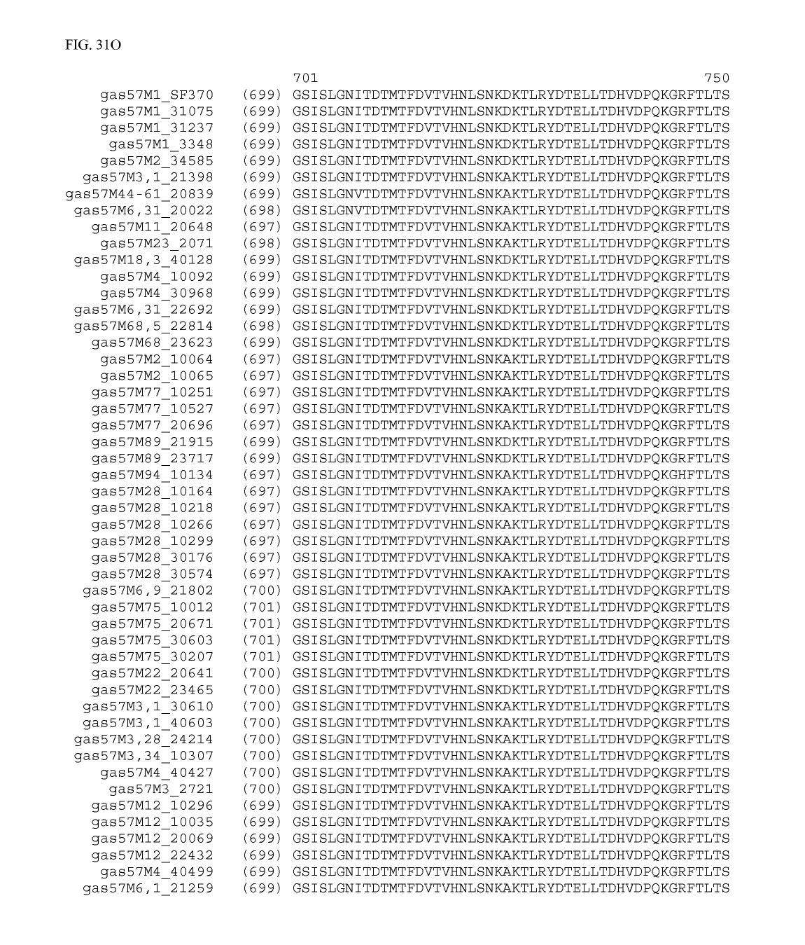

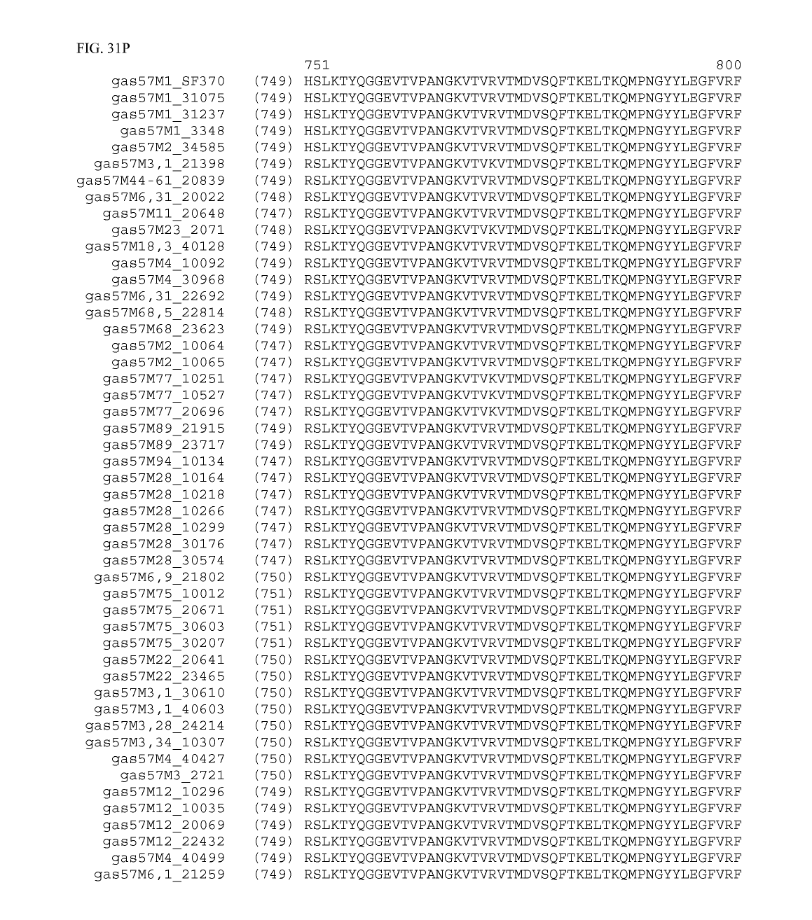

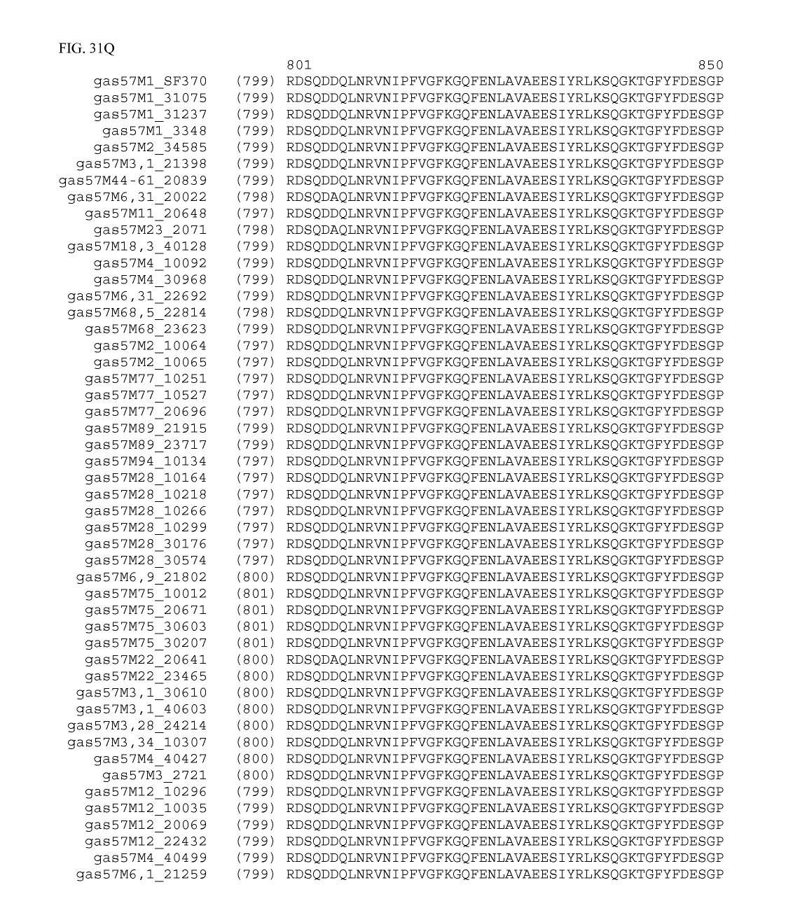

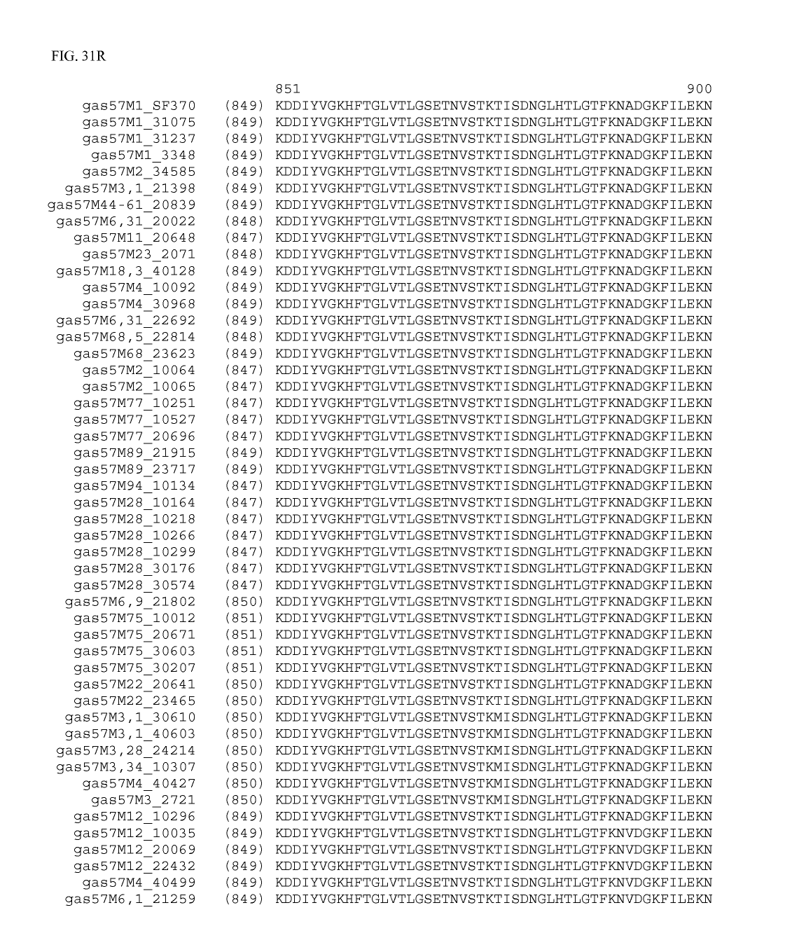

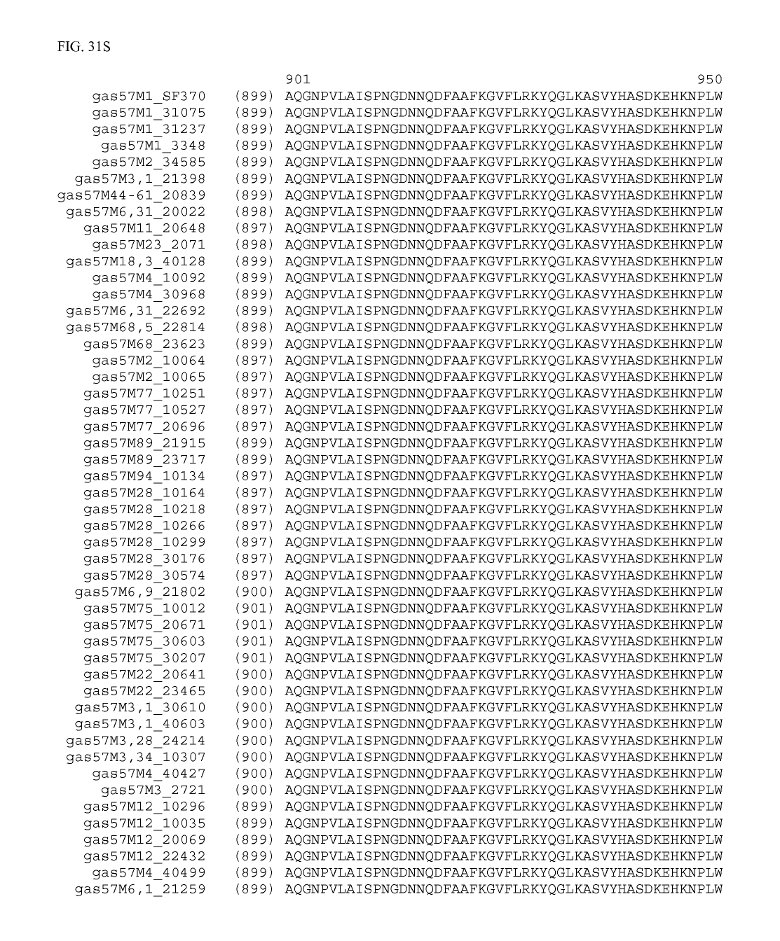

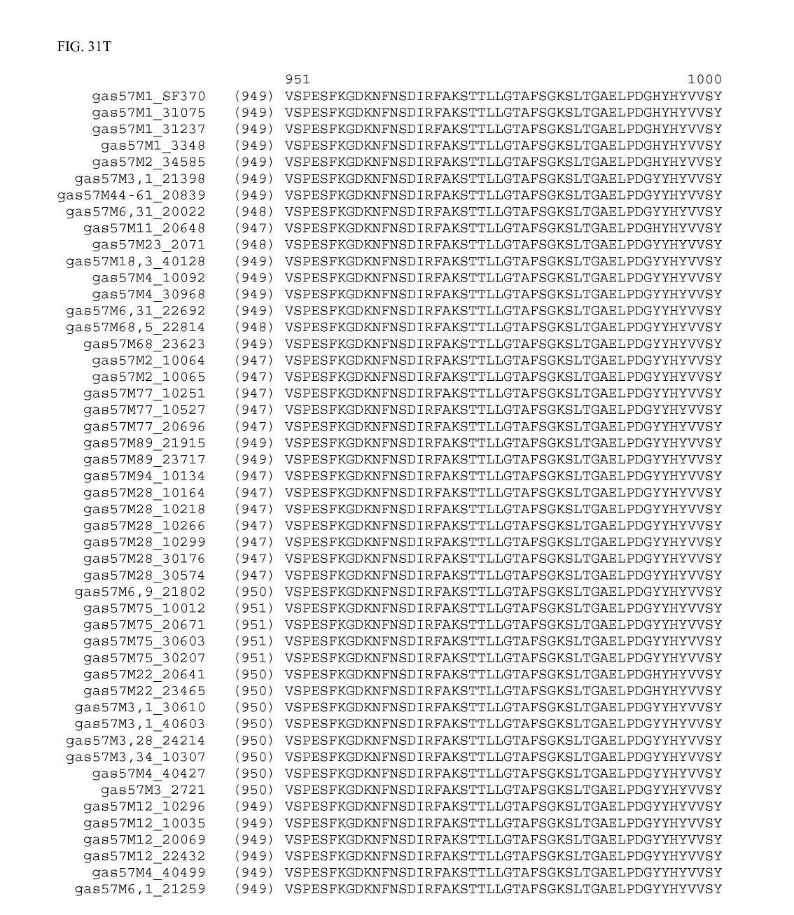

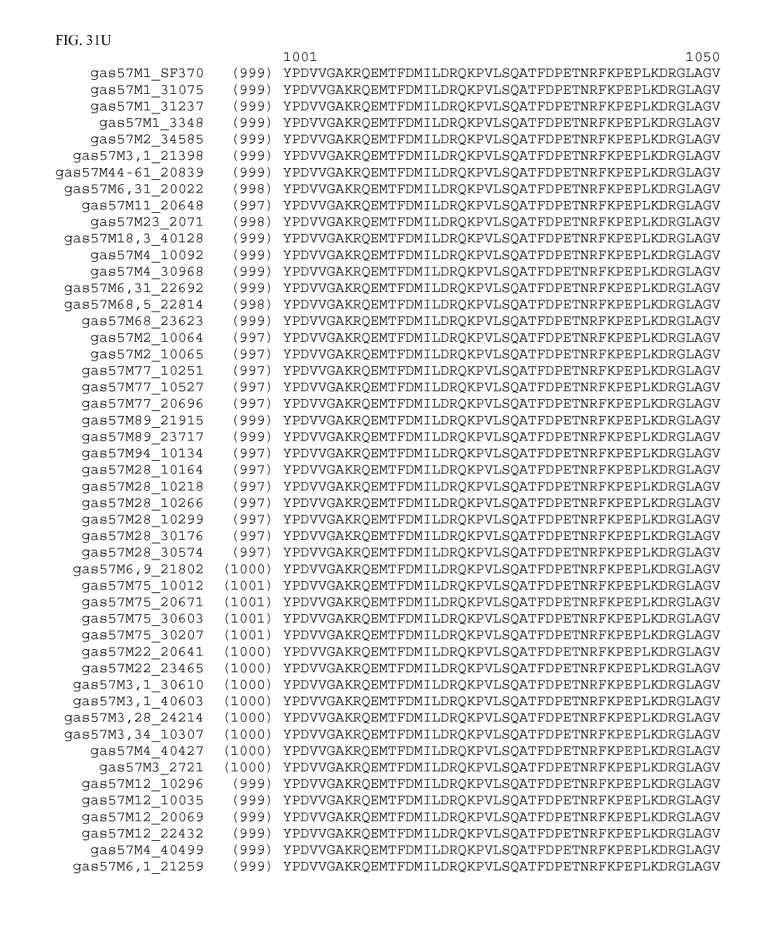

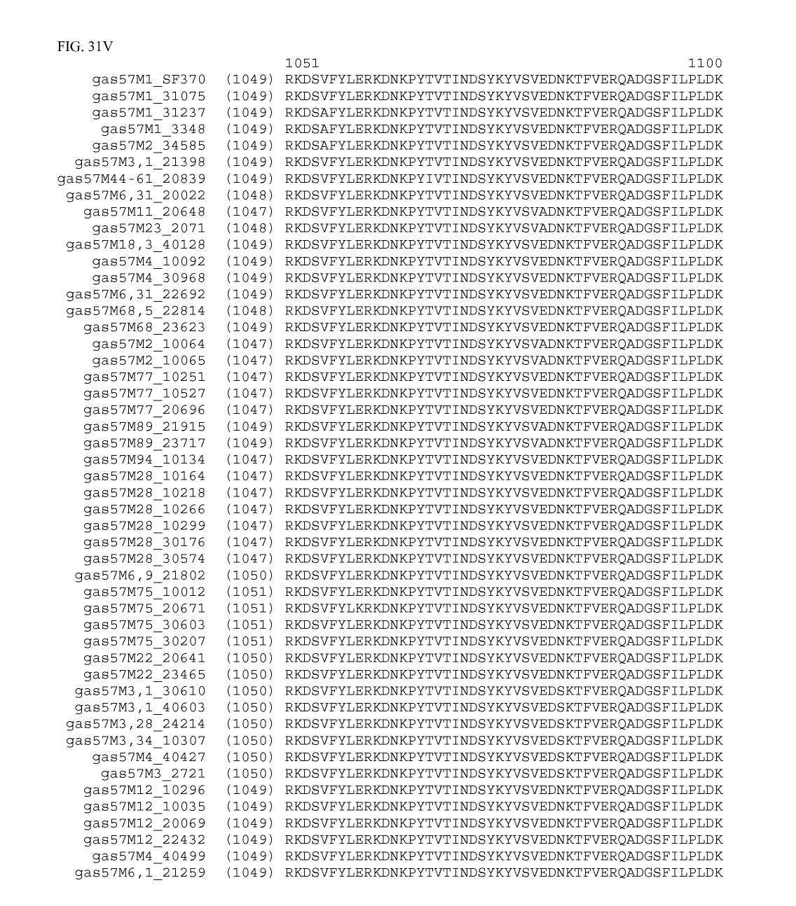

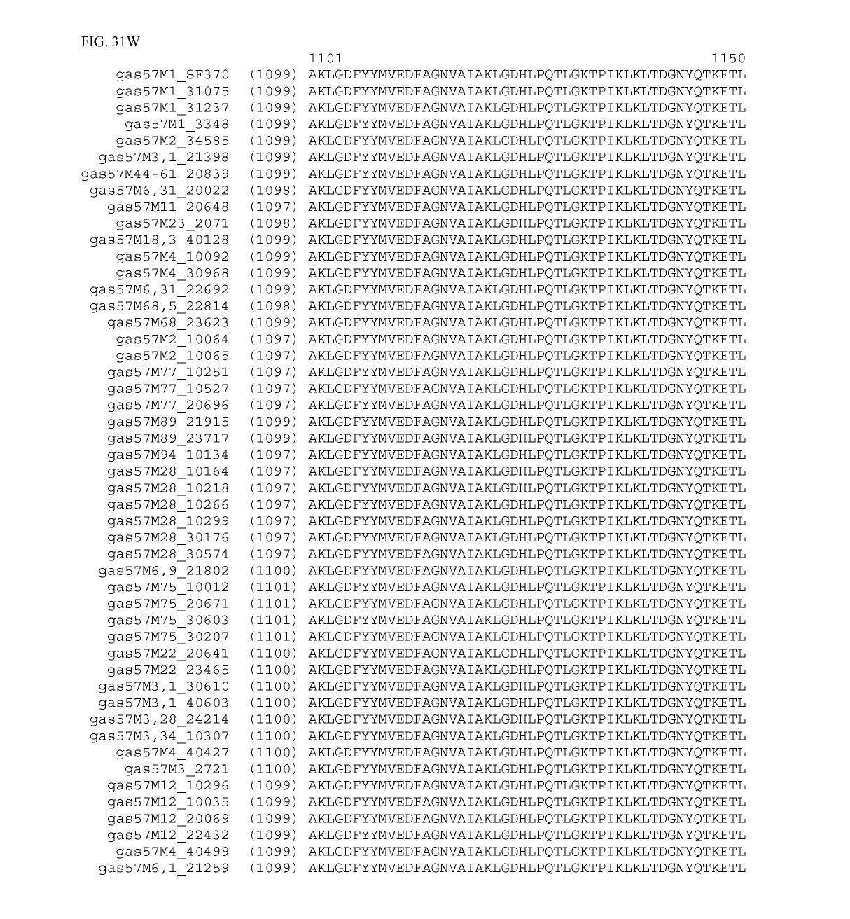

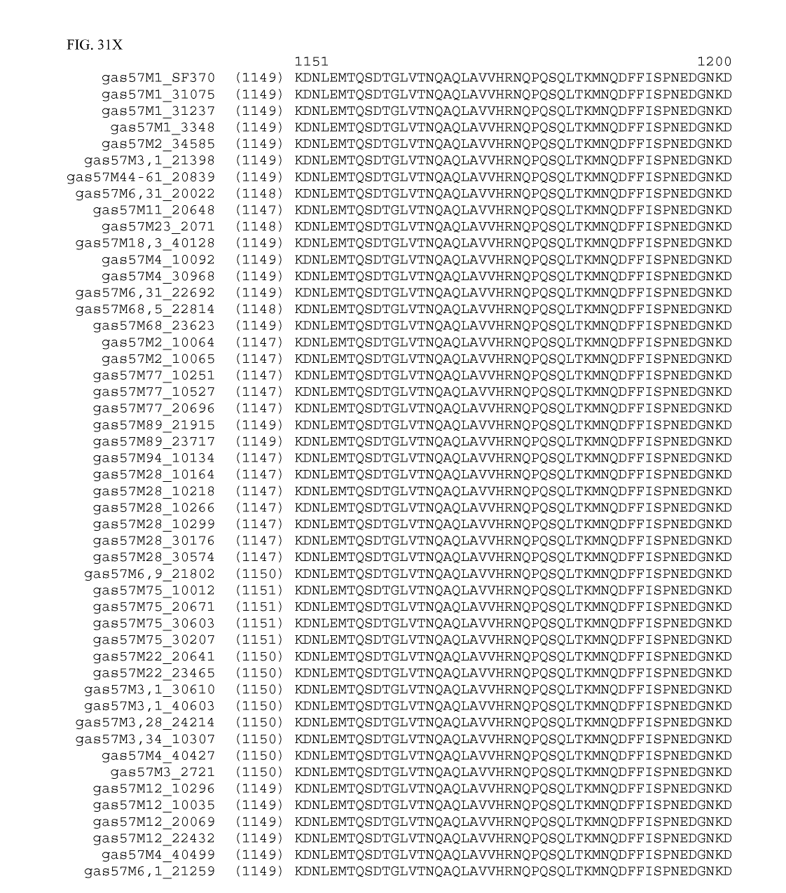

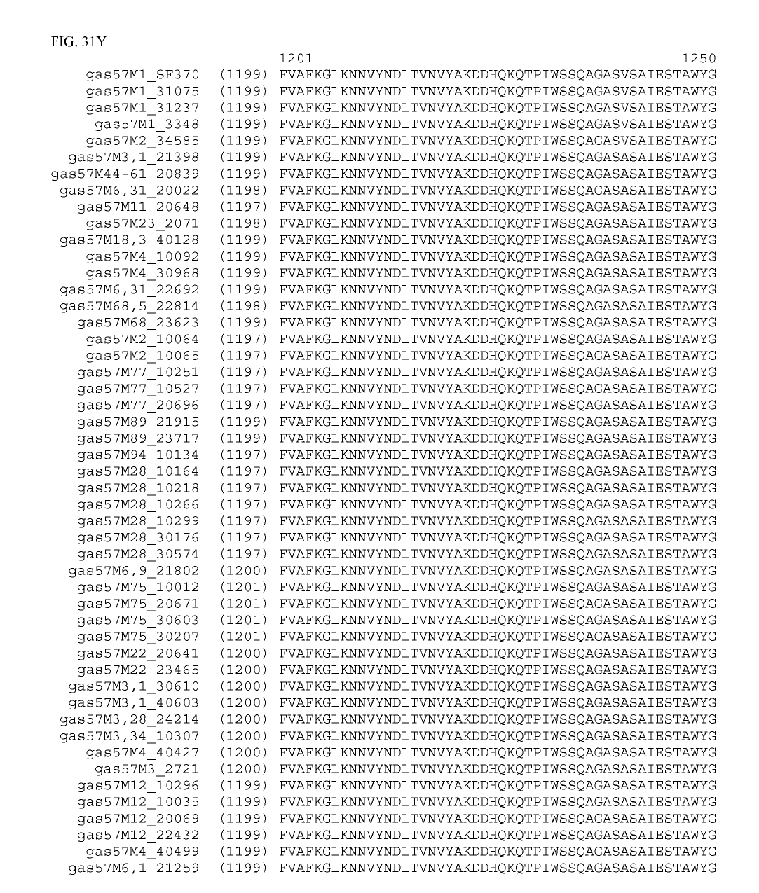

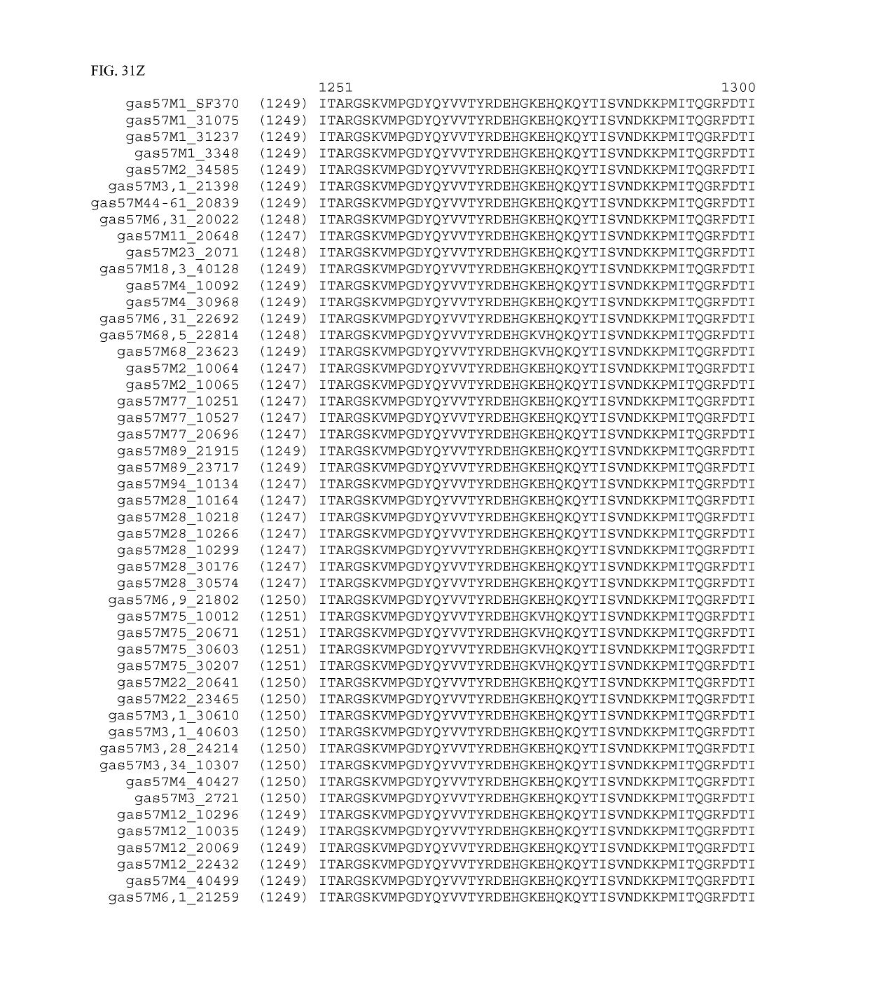

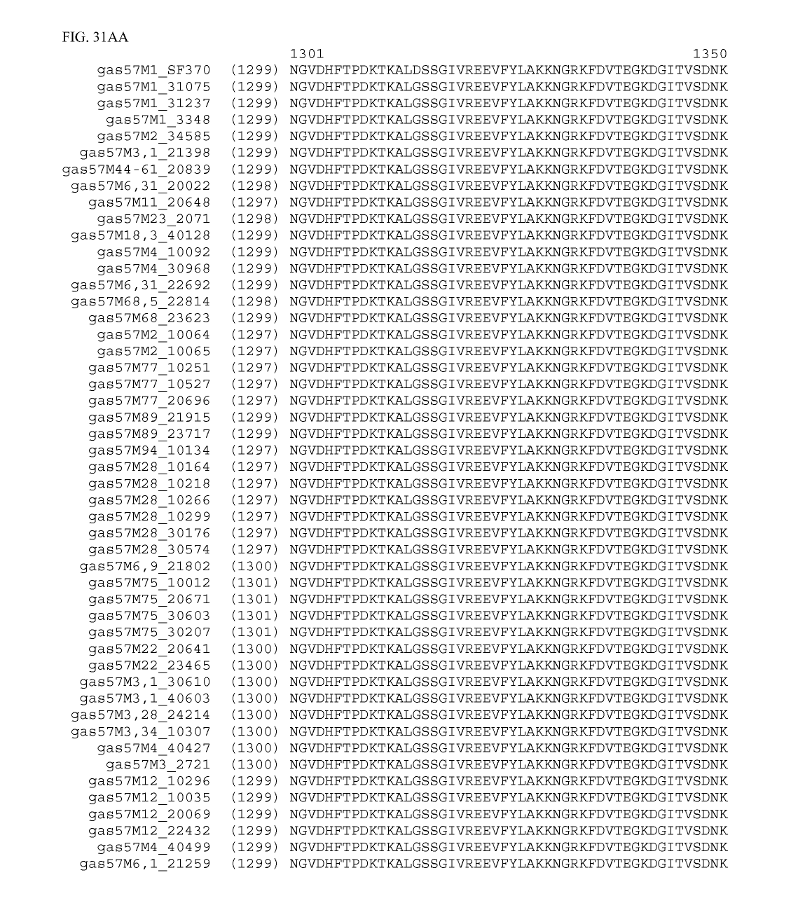

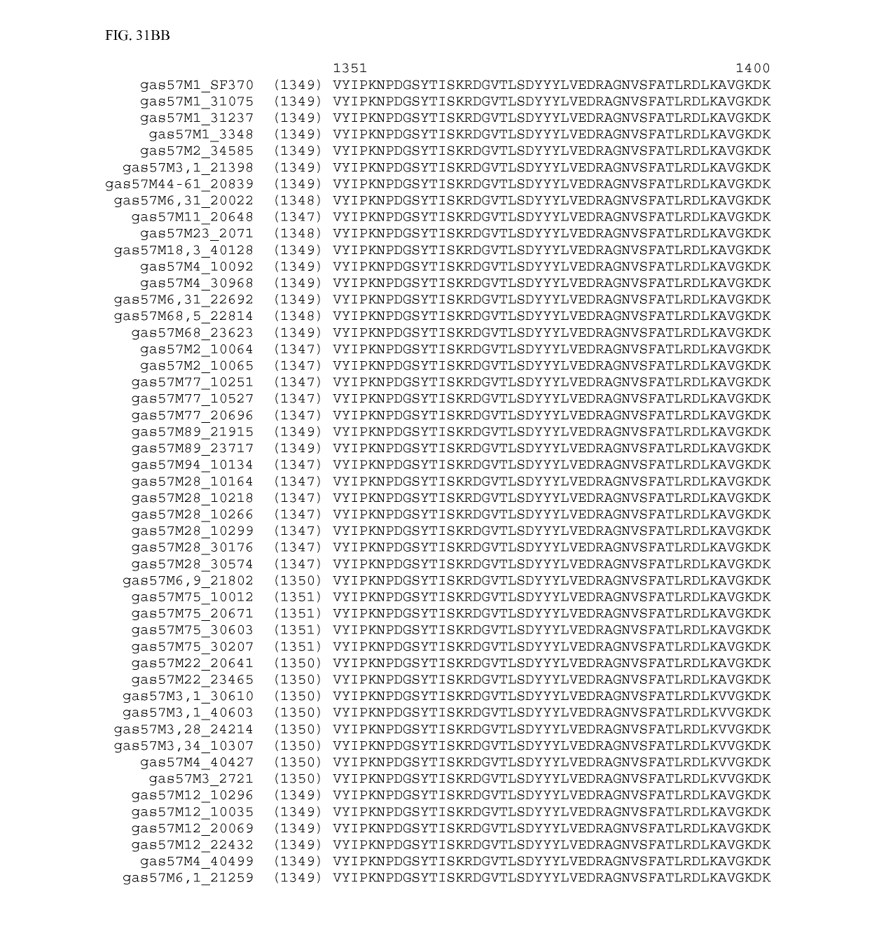

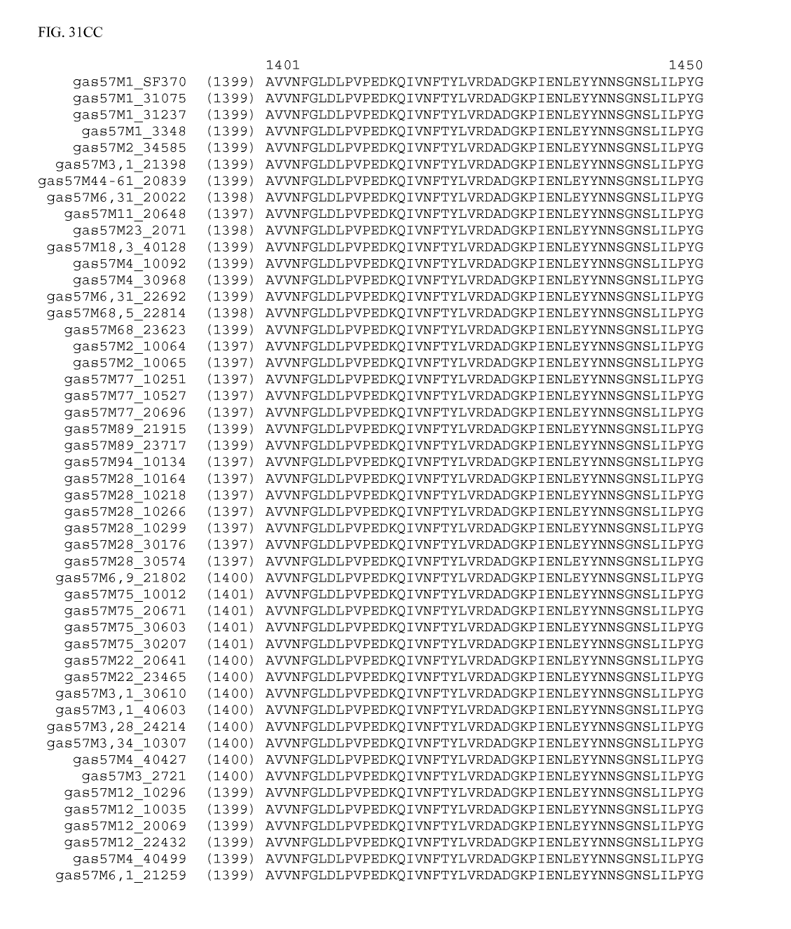

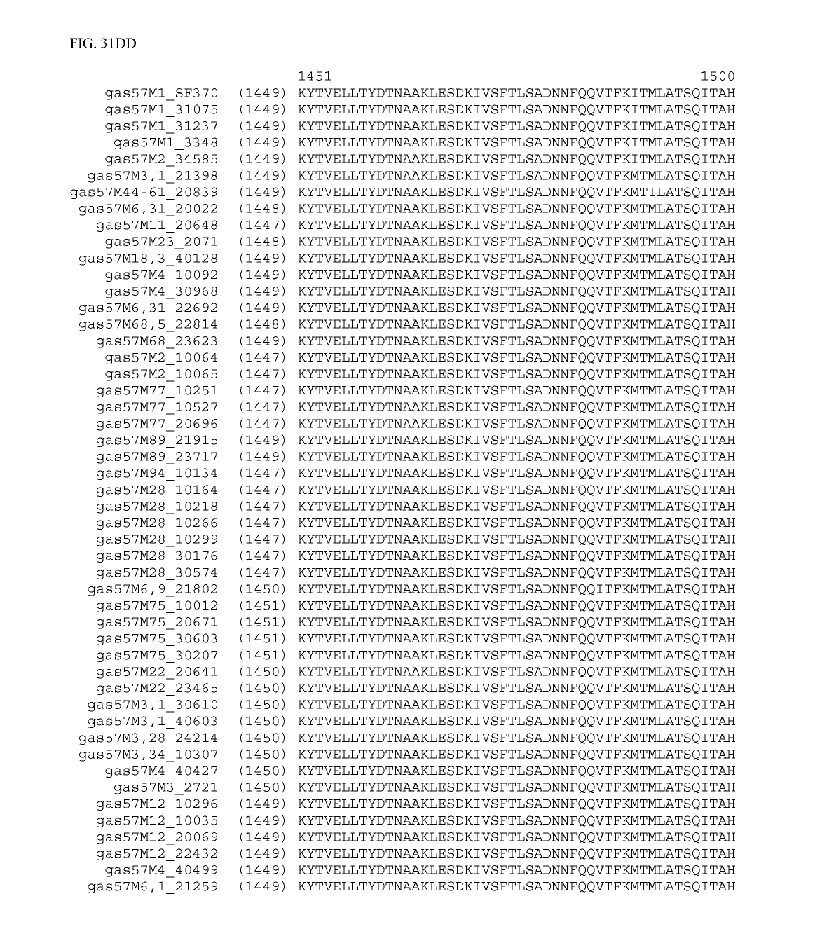

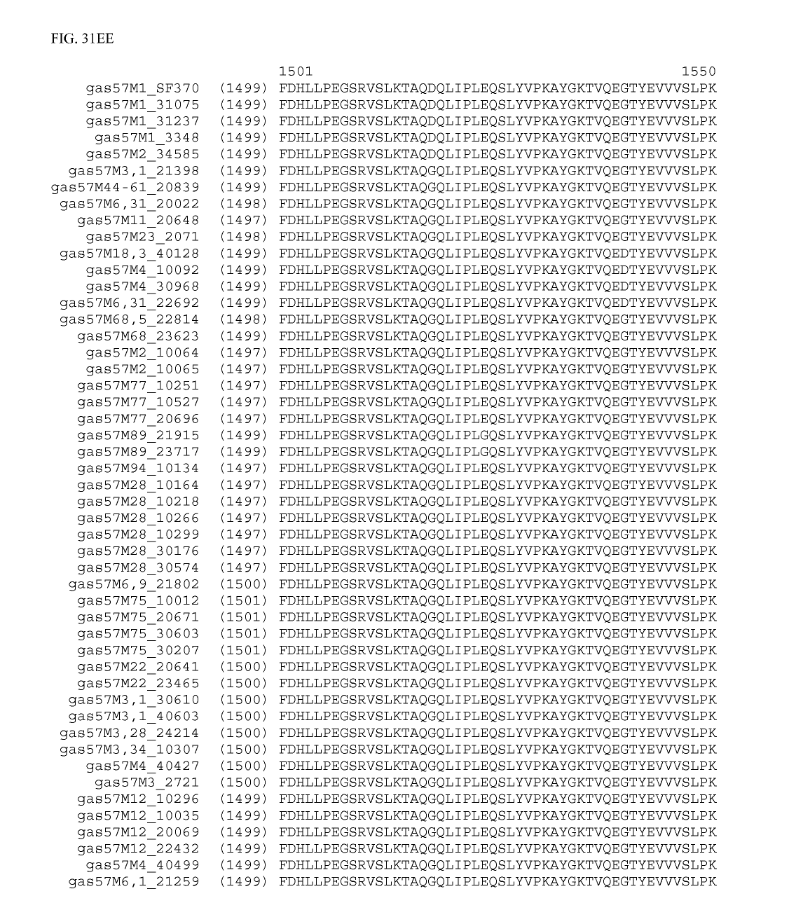

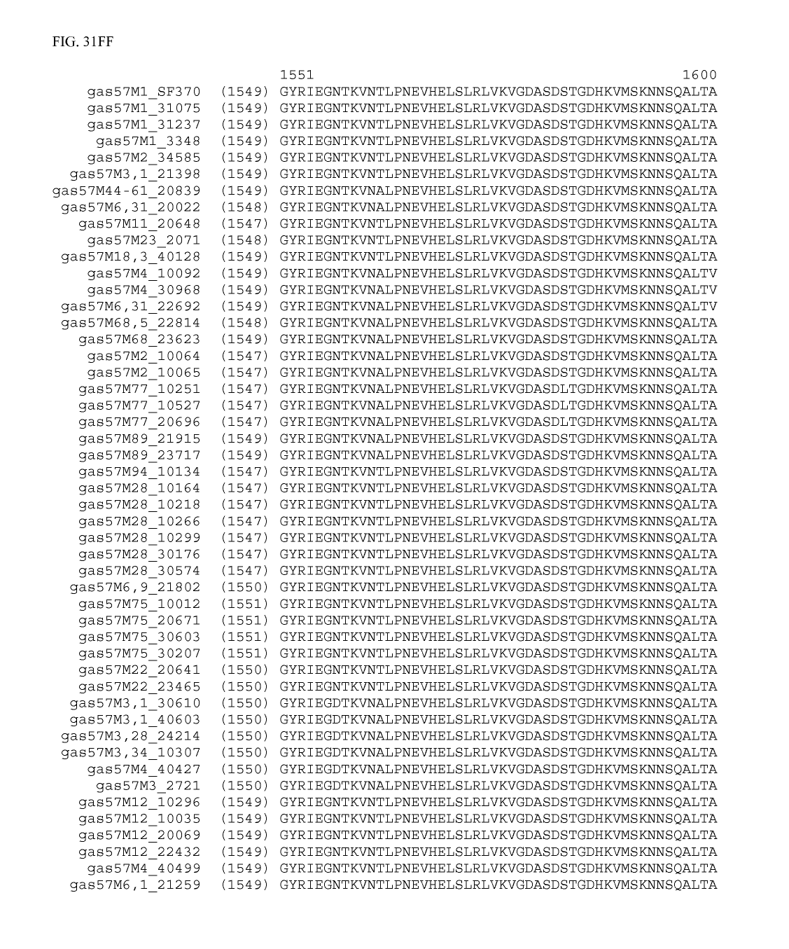

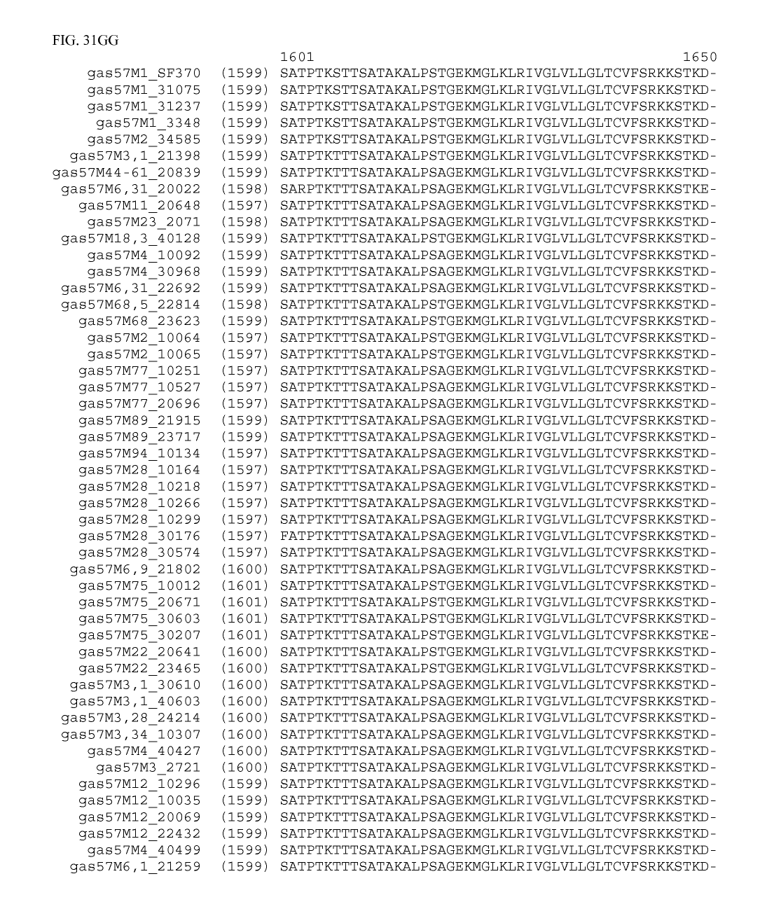

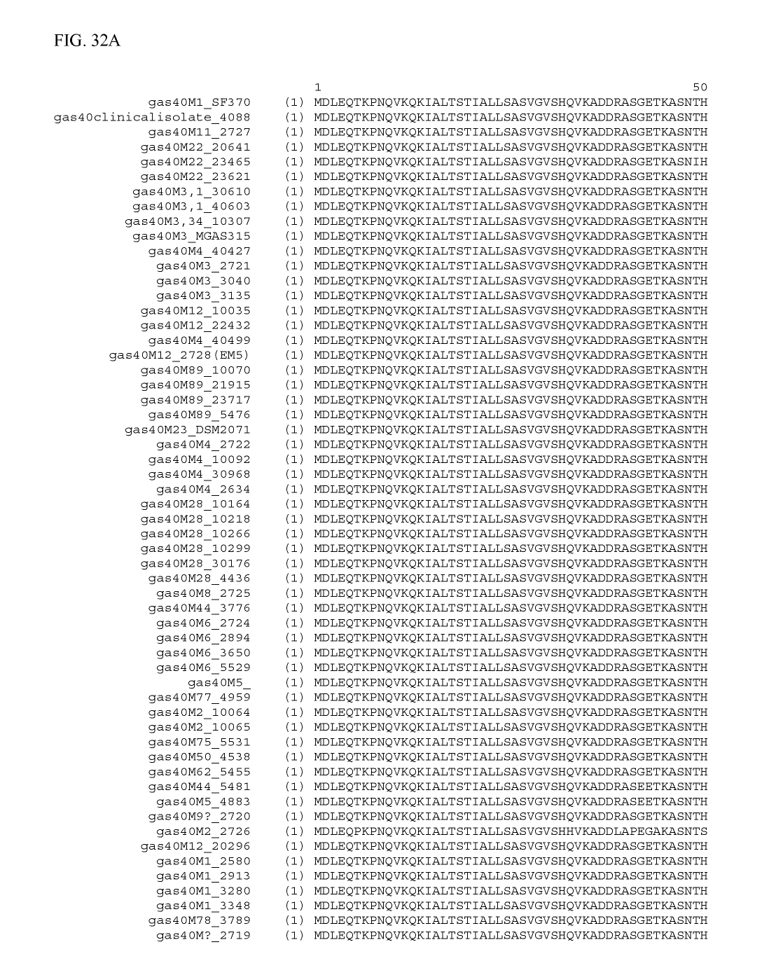

As there is variance among wild-type GAS antigens between GAS M types and GAS strain isolates, references to the GAS amino acid or polynucleotide sequences herein include equivalent amino acid or polynucleotide sequences having some degree of sequence identity thereto, typically because of conservative amino acid substitutions (see Example 30 and FIGS. 31-33.

In some embodiments, variants of Spy0167, Spy0269, Spy0416, Spy0714, Spy1390, Spy2000, and disclosed mutants thereof have amino acid sequences which are at least 90%, 91%, 92%, 93%, 94%, 95%, 96%, 97%, 98%, or 99% identical to any of the Spy0167, Spy0269, Spy0416, Spy0714, Spy1390, Spy2000 amino acid sequences disclosed herein, respectively. Typically any difference between the amino acid sequence of a GAS antigen and the amino acid sequence of a variant of the GAS antigen is due to one or more conservative amino acid substitutions. As indicated in FIG. 31 for example, one, two, or three amino acid deletions also are possible.

In some embodiments conservative amino acid substitutions are based on chemical properties and include substitution of a positively-charged amino acid for another positively charged amino acid (e.g., H, K, R); a negatively-charged amino acid for another negatively charged amino acid (e.g., D, E); a very hydrophobic amino acid for another very hydrophobic amino acid (e.g., C, F, I, L, M, V, W); a less hydrophobic amino acid for another less hydrophobic amino acid (e.g., A, G, H, P, S, T, Y); a partly hydrophobic amino acid for another partly hydrophobic amino acid (e.g., K, R); an aliphatic amino acid for another aliphatic amino acid (e.g., A, I, L, M, P, V); a polar amino acid for another polar amino acid (e.g., A, D, E, G, H, K, N, P, Q, R, S, T, Y); an aromatic amino acid for another aromatic amino acid (e.g., F, H, W, Y); and a small amino acid for another small amino acid (e.g., D, N, T).

In some embodiments, conservative amino acid substitutions are determined using the BLOSUM62 table. The BLOSUM62 table is an amino acid substitution matrix derived from about 2,000 local multiple alignments of protein sequence segments, representing highly conserved regions of more than 500 groups of related proteins (Henikoff & Henikoff, Proc. Natl. Acad. Sci. USA 89:10915, 1992). The BLOSUM62 substitution frequencies can be used to define conservative amino acid substitutions that may be introduced into amino acid sequences of Spy0167, Spy0269, Spy0416, Spy0714, Spy1390, and Spy2000 antigens. In these embodiments a conservative substitution preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

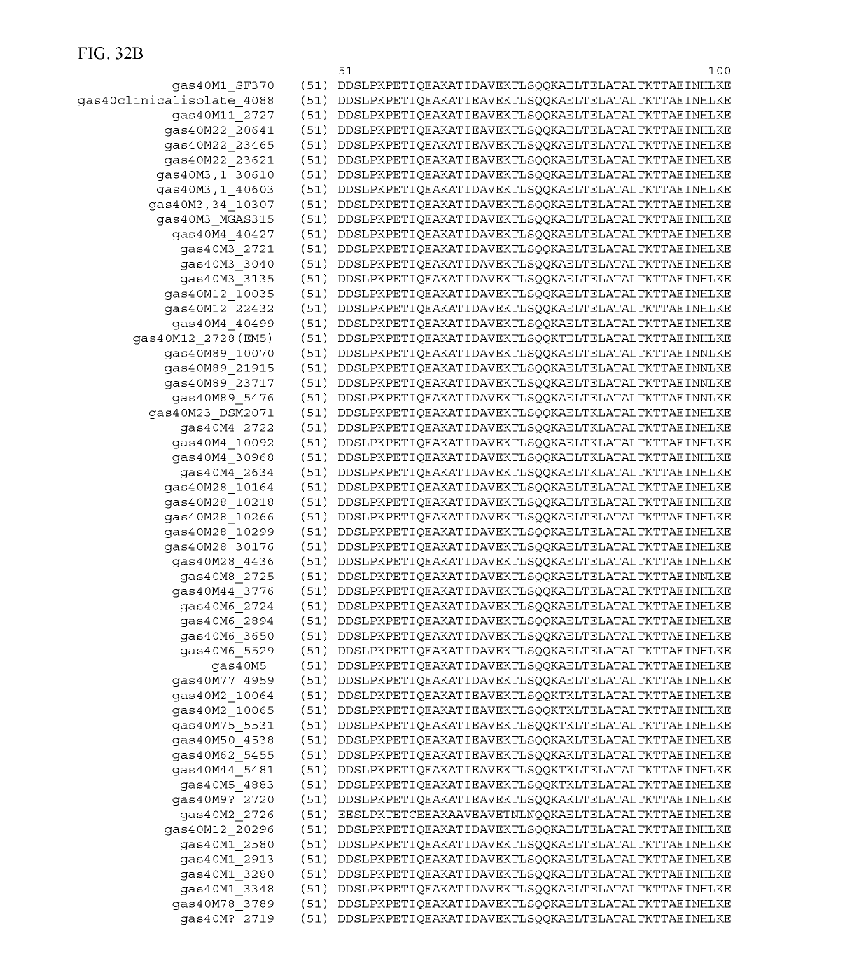

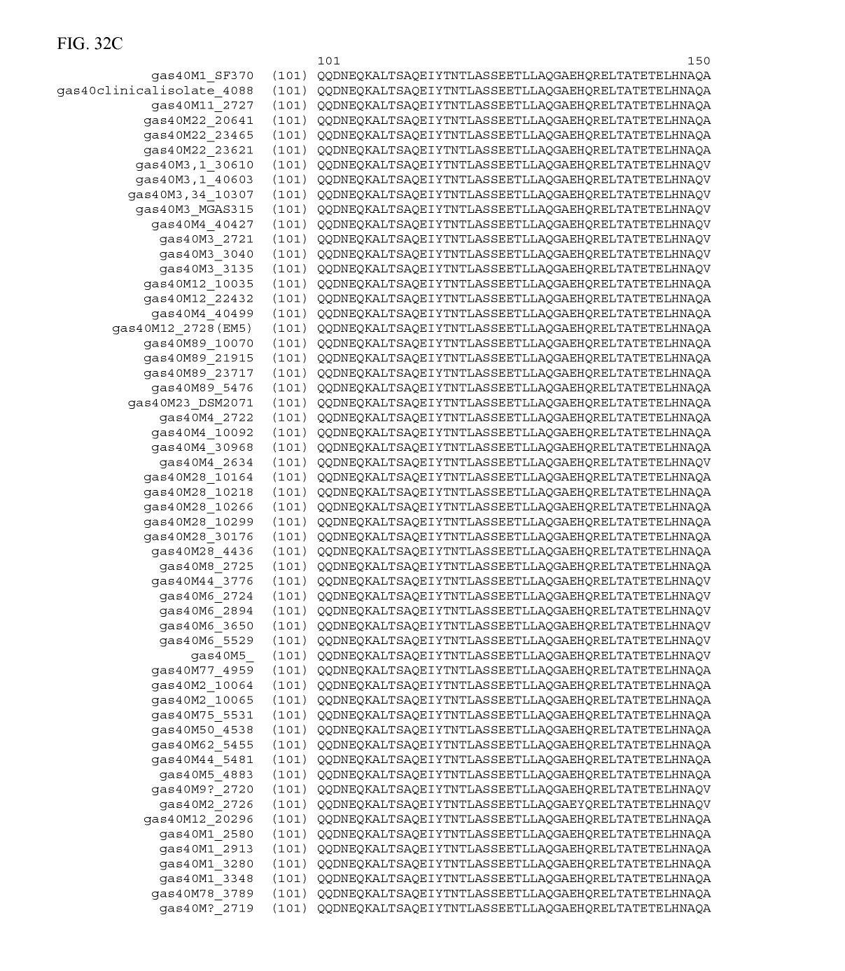

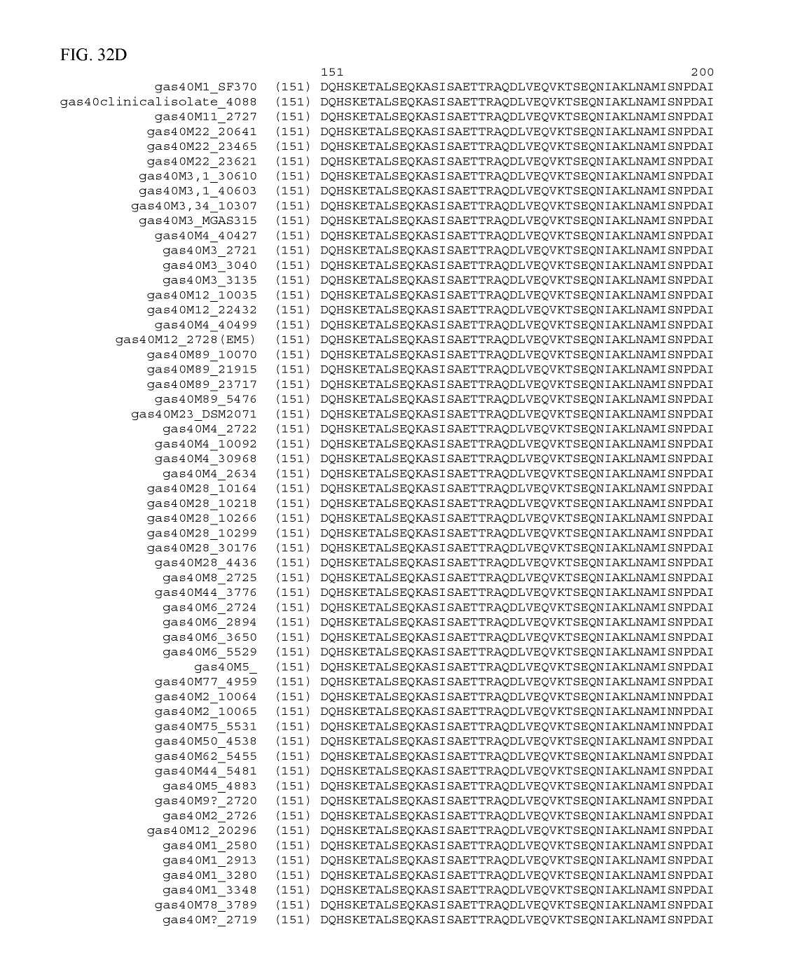

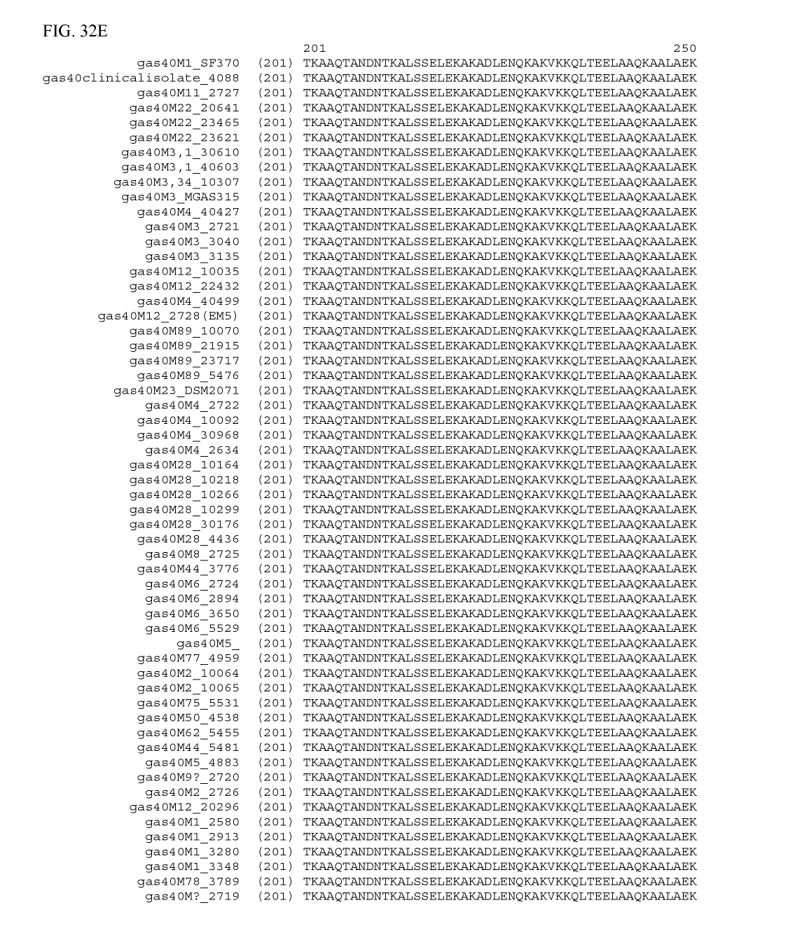

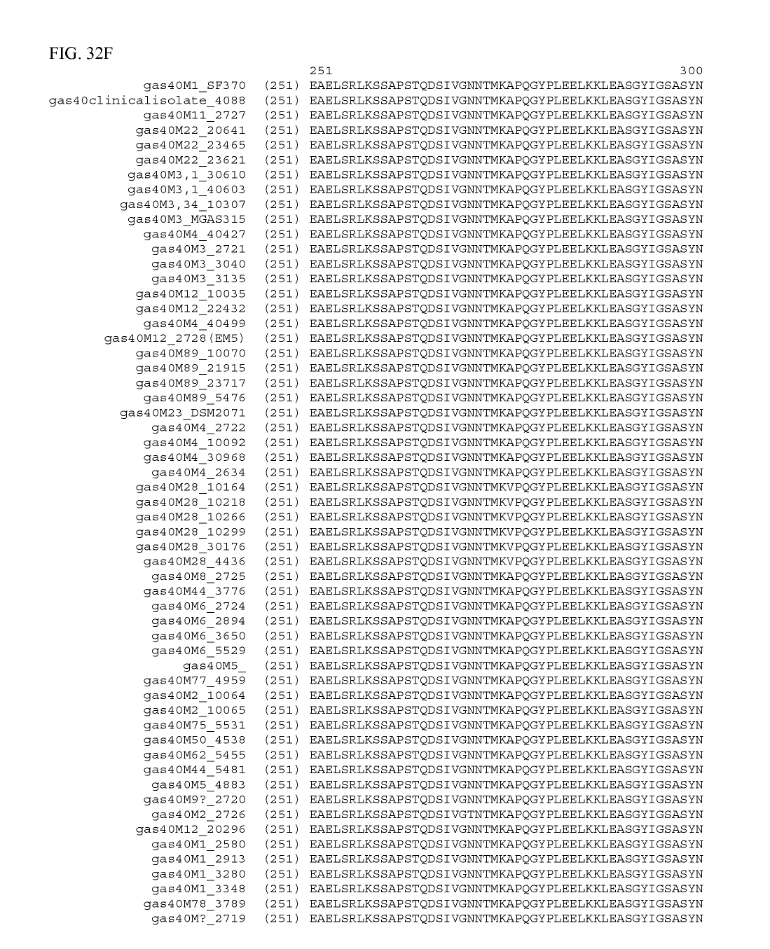

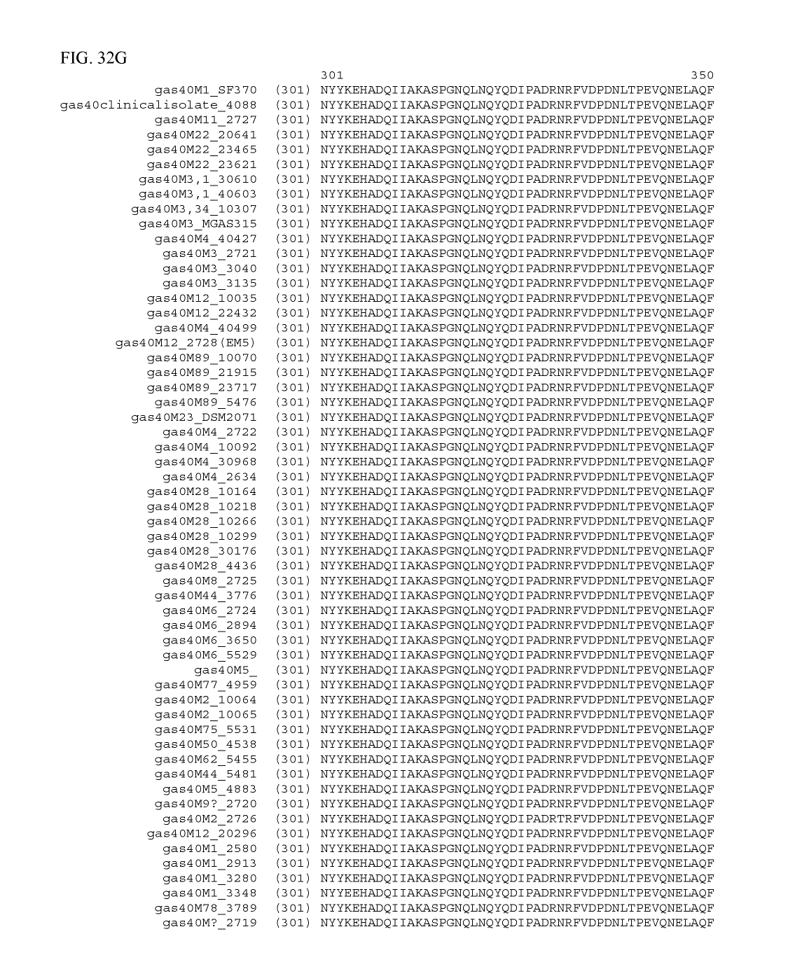

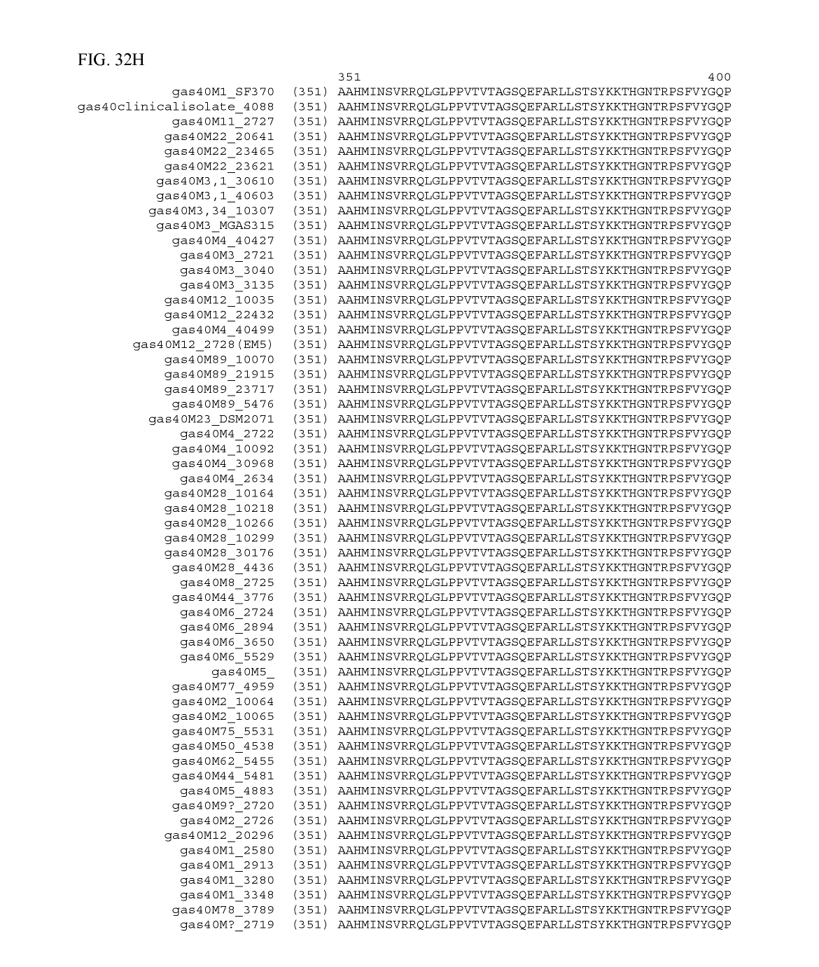

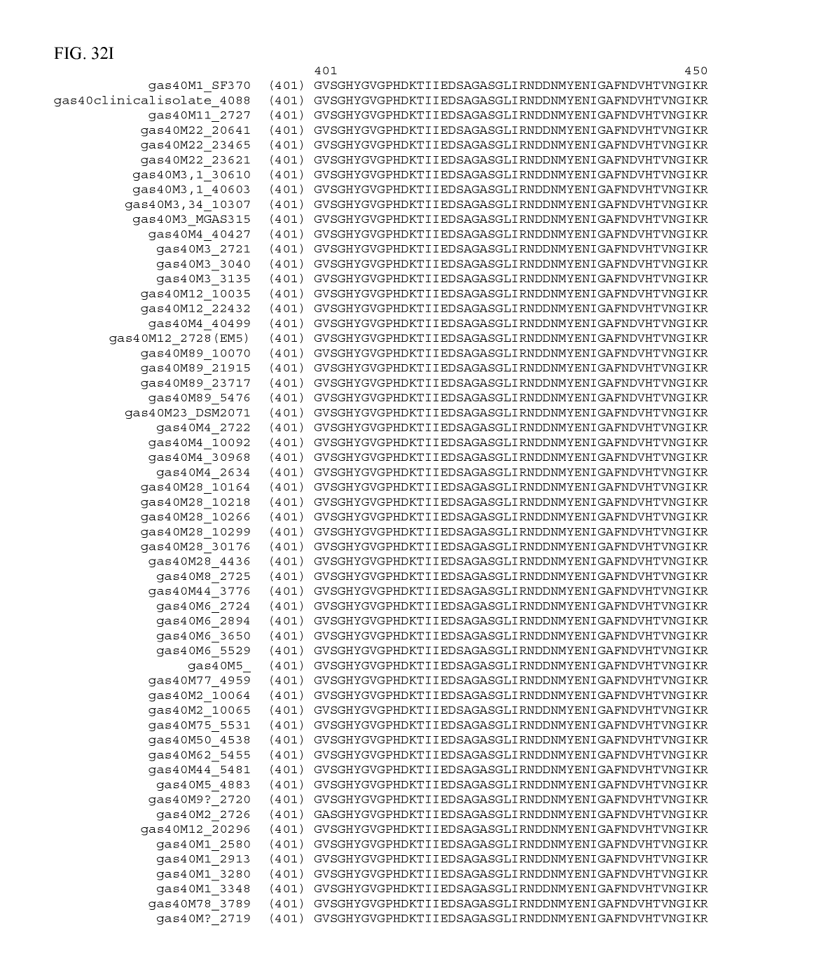

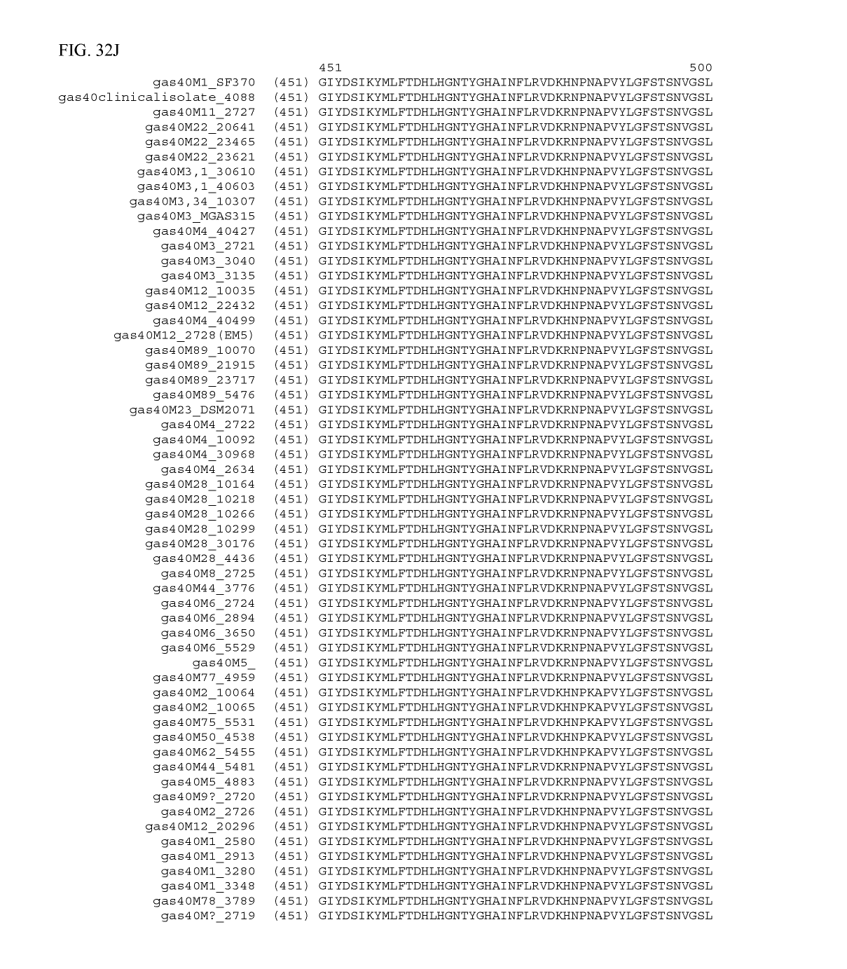

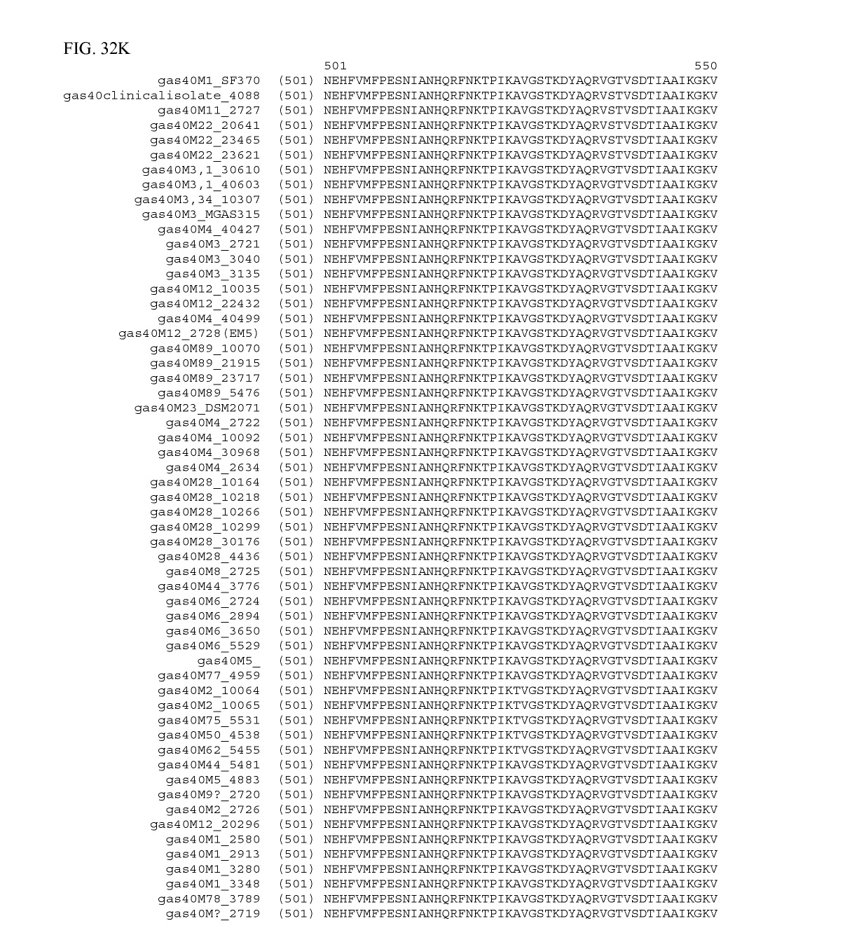

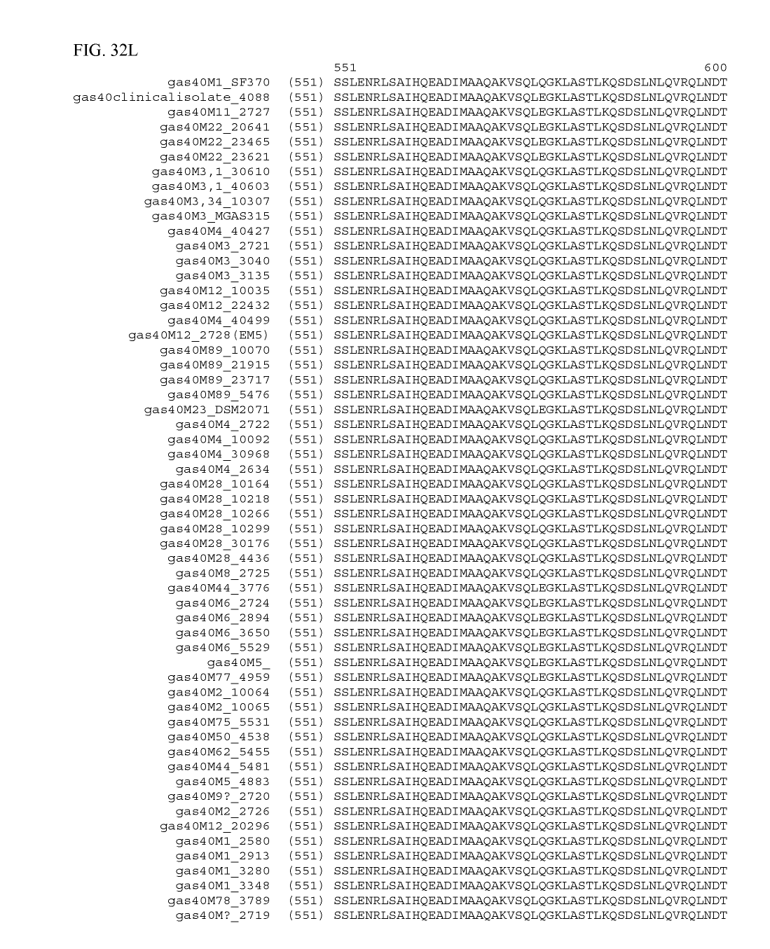

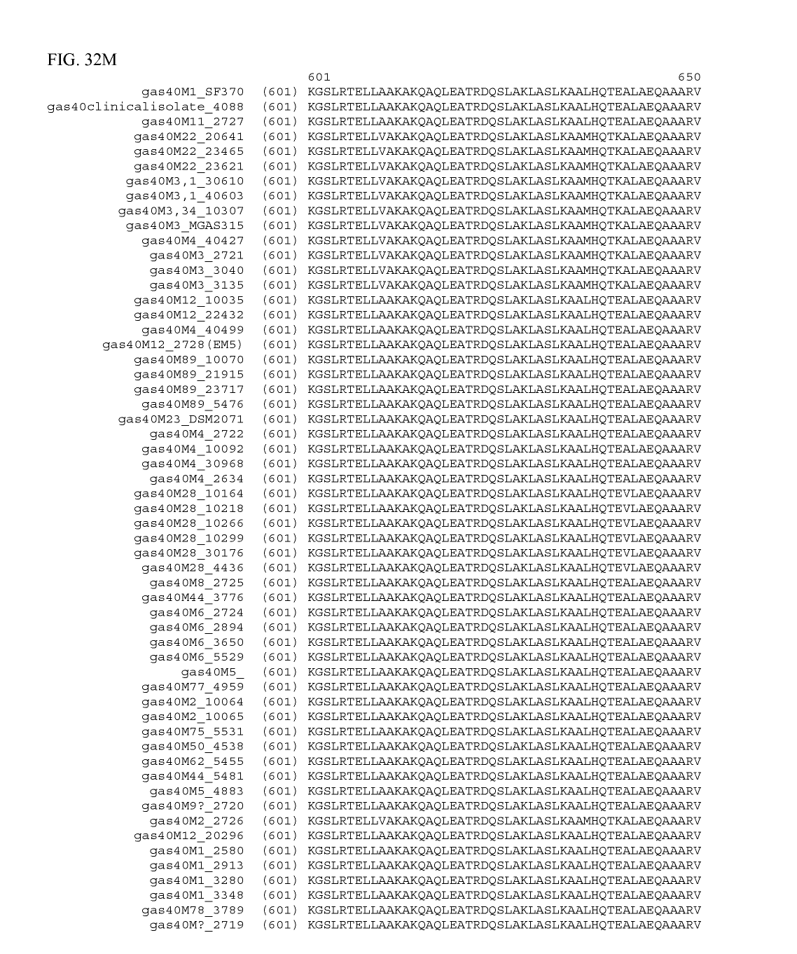

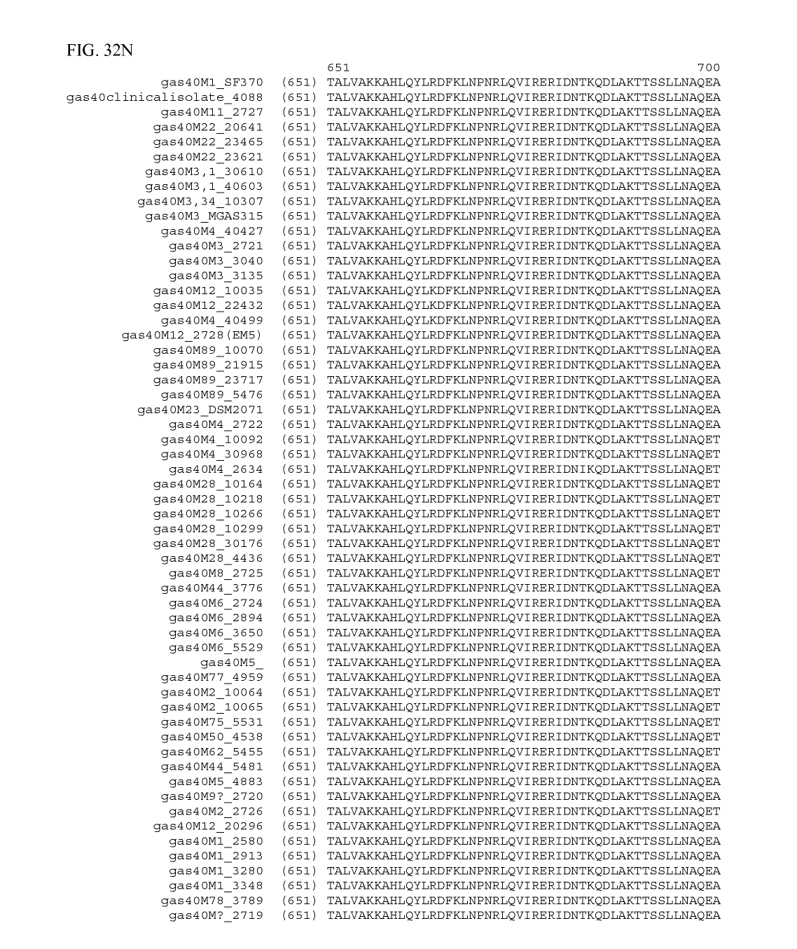

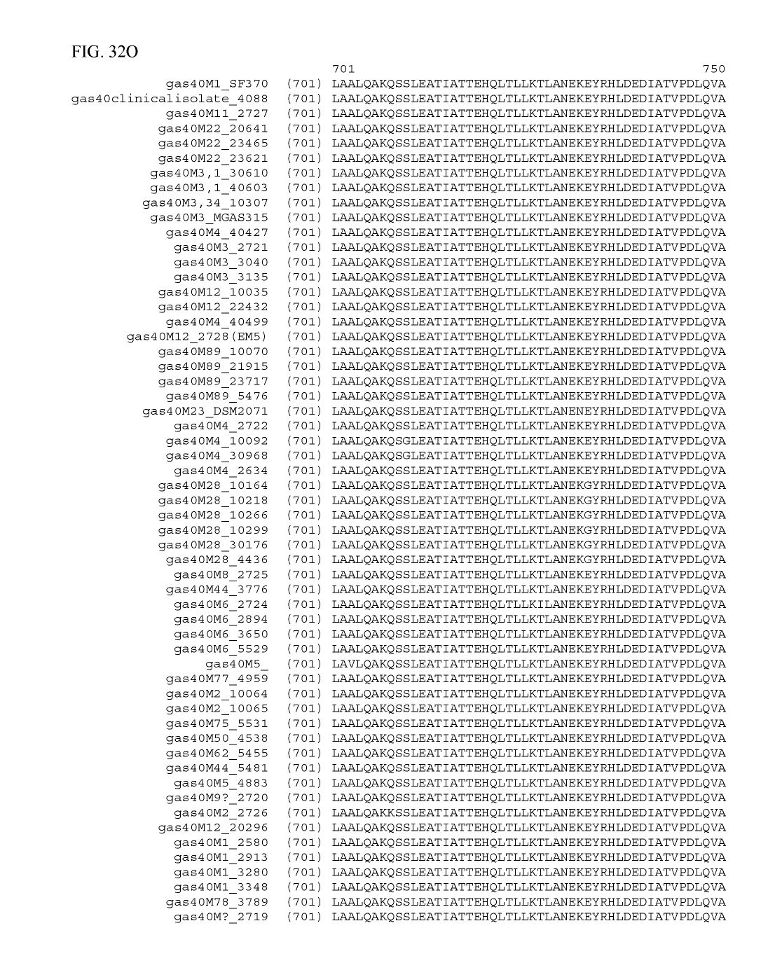

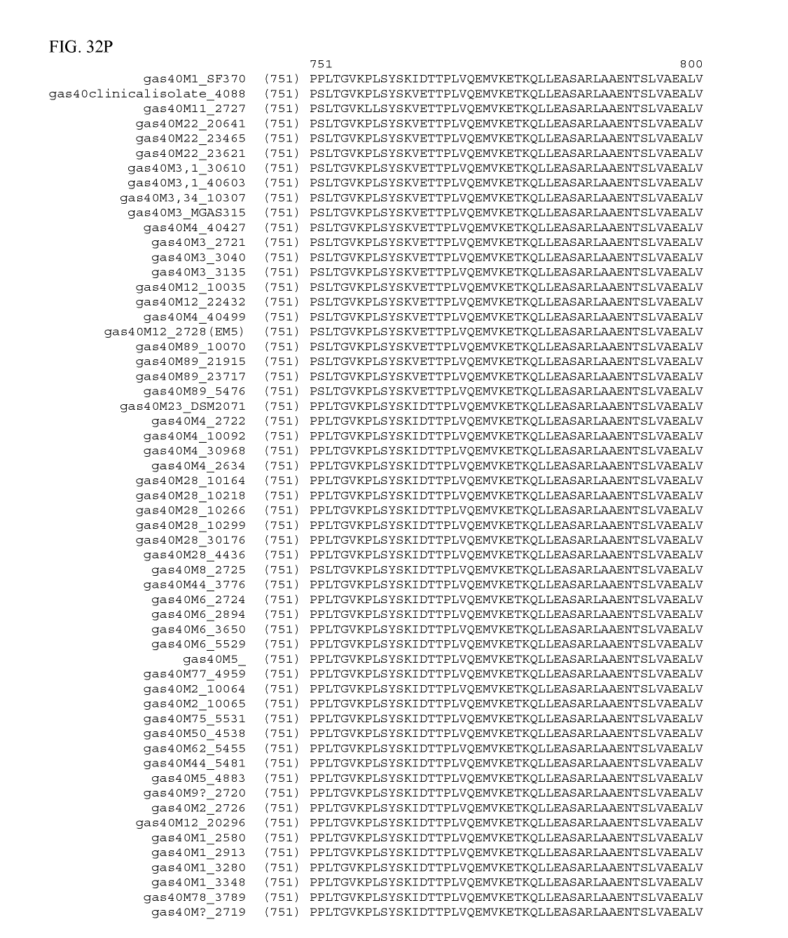

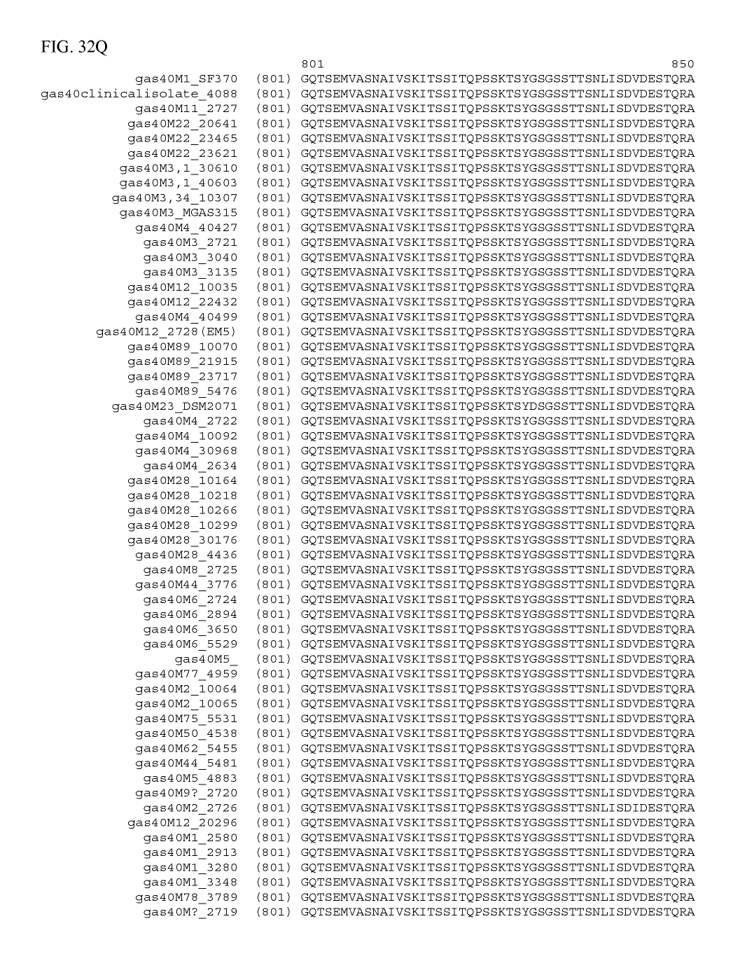

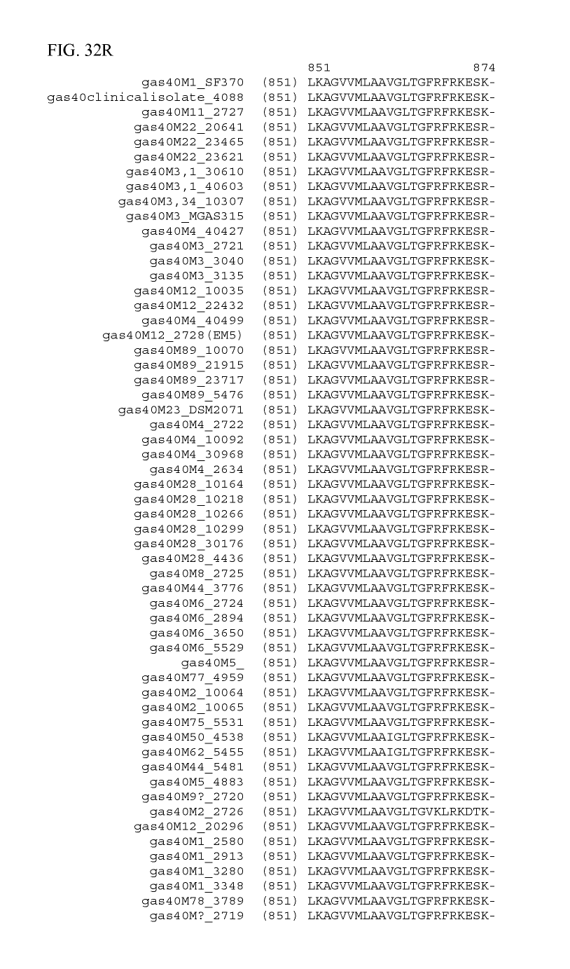

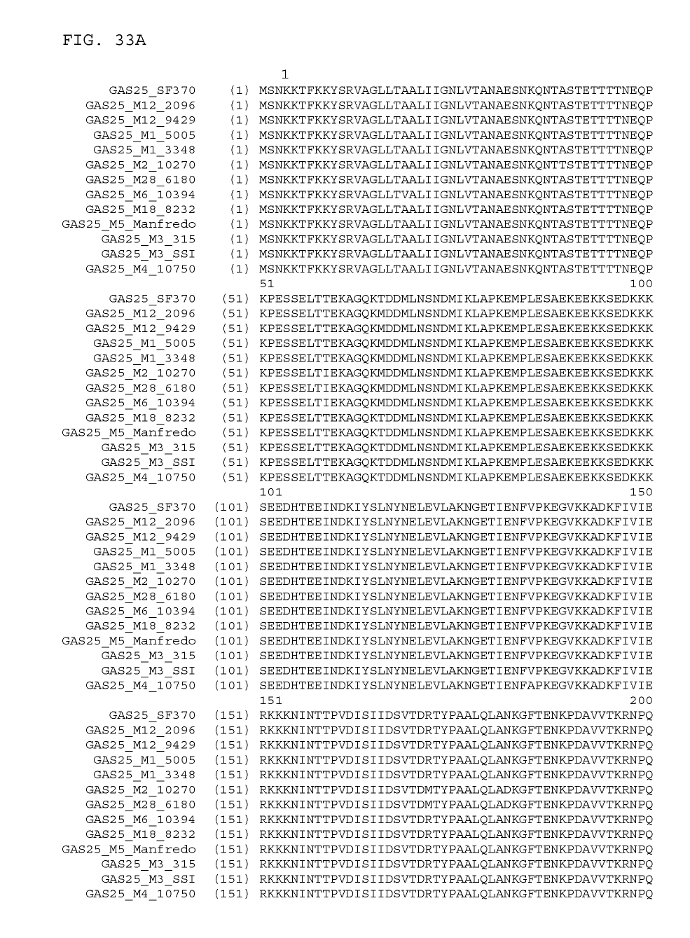

Particular amino acid substitutions or alterations can be identified by aligning the various Spy0167, Spy0269, Spy0416, Spy0714, Spy1390, and Spy2000 amino acid sequences as shown for wild-type Spy0416 in FIG. 31, wild-type Spy0269 in FIG. 32, and wild-type Spy0167 in FIG. 33. For example, based on the alignment in FIG. 31, Table 1 indicates some particular options at certain amino acid positions with respect to the Spy0416 amino acid sequence shown in SEQ ID NO: 1. Similarly, options for amino acid variations in the amino acid sequences of Spy0269 and Spy0167 can be identified by inspection of FIGS. 32 and 33, respectively.

TABLE 1

| position | options |
|---|---|
| 38 | S, T |
| 40 | M, S, T |
| 49 | A, T |
| 55 | H, P |
| 67 | K, Q |
| 68 | S, P |
| 69 | Q, P |
| 74 | I, V |
| 77 | E, K |
| 85 | S, P |
| 87 | D, G |
| 91 | E, K |
| 93 | T or missing |
| 102 | A, S |
| 104 | S, P |
| 110 | S, P |
| 228 | A or missing |
| 229 | D, E, or missing |
| 749 | H, R |

Variants of the GAS antigens described below preferably are immunogenic and confer protection against GAS lethal challenge in a mouse model (see the Examples, below).

In some embodiments compositions comprise one or more nucleic acid molecules encoding the GAS protein antigens disclosed above. In other embodiments compositions comprise no more than two nucleic acid molecules encoding two GAS protein antigens. In other embodiments compositions comprise combinations of antibodies, wherein each antibody selectively binds to a GAS antigen selected from the GAS protein antigens disclosed above.

### Spy0167 and Immunogenic Mutants Thereof

Spy0167 (streptolysin, SLO, GAS25) is a potent pore-forming toxin which induces host cell lysis and is described, inter alia, in WO 02/34771. Amino acid sequences for wild-type Spy0167 are shown in SEQ ID NOS:107-119. Unless otherwise defined, a “Spy0167 antigen” includes full-length Spy0167 and Spy0167 mutants, fragments, and fusions, as described below.

In some embodiments a Spy0167 antigen consists essentially of the amino acid sequence SEQ ID NO:174 (“peptide 1”), the amino acid sequence SEQ ID NO:175 (“peptide 2”), or the amino acid sequence SEQ ID NO:176 (“peptide 3”). In some embodiments a Spy0167 antigen consists essentially of, from N to C terminus, the amino acid sequence SEQ ID NO:175 (“peptide 2”) and the amino acid sequence SEQ ID NO:176 (“peptide 3”) covalently attached to the amino acid sequence SEQ ID NO:175. “Covalently attached” as used herein includes direct covalent linkage as well as linkage via one or more additional amino acids. In other embodiments a Spy0167 antigen consists essentially of, from N to C terminus, the amino acid sequence SEQ ID NO:174; a glycine residue covalently attached to the amino acid sequence SEQ ID NO:174; the amino acid sequence SEQ ID NO:175 covalently attached to the glycine; and the amino acid sequence SEQ ID NO:176 covalently attached to the amino acid sequence SEQ ID NO:175.

Other Spy0167 antigens are fragments of Spy0167 which are less than full-length Spy0167 by at least one amino acid. Preferably the fragments retain an immunological property of the antigen, such as the ability to bind specific antibodies. Preferred amino acid fragments comprise 7 or more amino acids (e.g., 8, 10, 12, 14, 16, 18, 20, 25, 30, 35, 40, 50 or more).

In some embodiments, a Spy0167 antigen is a monomer which comprises the amino acid sequence SEQ ID NO:174. In other embodiments a Spy0167 antigen is a monomer which comprises, from N to C terminus, the amino acid sequence SEQ ID NO:175 and the amino acid sequence SEQ ID NO:176 covalently attached to the amino acid sequence SEQ ID NO:175. In other embodiments a Spy0167 antigen is a monomer which comprises, from N to C terminus, the amino acid sequence SEQ ID NO:174; a glycine residue covalently attached to the amino acid sequence SEQ ID NO:174; the amino acid sequence SEQ ID NO:175 covalently attached to the glycine; and the amino acid sequence SEQ ID NO:176 covalently attached to the amino acid sequence SEQ ID NO:175.

Fusion Proteins

As disclosed above, Spy0167 antigens used in the invention may be present in the composition as individual separate polypeptides (e.g., “peptide 1,” “peptide 2,” “peptide 3,” “peptide 1+2+3,” “peptide 2+3”), but there also are embodiments in which at least two (i.e., 2, 3, 4, 5, 6, 7, 8, 9, 10, 11, 12, 13, 14, 15, 16, 17, 18, 19 or 20) antigens are expressed as a single polypeptide chain (a “fusion protein” or “hybrid polypeptide”). Hybrid polypeptides offer two principal advantages. First, a polypeptide that may be unstable or poorly expressed on its own can be assisted by adding a suitable hybrid partner that overcomes the problem. Second, commercial manufacture is simplified as only one expression and purification need be employed in order to produce two polypeptides which are both antigenically useful.

Mutant Forms of Spy0167

Mutant forms of Spy0167 have at least 50% less hemolytic activity than wild-type Spy0167 (e.g., 50, 55, 60, 65, 70, 75, 80, 85, 90, 95, 96, 97, 98, 99, or 100%) relative to wild-type Spy0167 as determined by a hemolysis assay (e.g., see Example 1) but are immunogenic and preferably confer protection against GAS lethal challenge in a mouse model (see Examples 4, 7, 8). Spy0167 mutants include those with an amino acid alteration (i.e., a substitution, deletion, or insertion) at one or more of amino acids P427, W535, C530, A248, and D482 numbered according to the wild-type Spy0167 sequence shown in SEQ ID NO:107. Examples of such mutants include P427L (SEQ ID NO:120), W535F (SEQ ID NO:121), C530G (SEQ ID NO:122), ΔA248 (SEQ ID NO:123), W535F/D482N (SEQ ID NO:124), P427L/W535F (SEQ ID NO:125), P427L/C530G (SEQ ID NO:126), and P427L/C530G/W535F (SEQ ID NO:127).

Spy0167 mutants for use in the invention include single, double, or triple amino acid alterations (“single mutants,” “double mutants,” “triple mutants”) at positions P427, W535, C530, A248, and/or D482. Thus, Spy0167 mutants can comprise the following:
i. P427L (SEQ ID NO:120), P427R, P427N, P427C, P427Q, P427E, P427G, P427H, P4271, P427L, P427K, P427M, P427F, P427A, P427S, P427T, P427W, P427Y, or P427V;
ii. W535F (SEQ ID NO:121), W535R, W535N, W535D, W535C, W535Q, W535E, W535G, W5351, W535L, W535K, W535M, W535A, W535P, W535S, W535T, W535Y, or W535V;
iii. C530G (SEQ ID NO:122), C530R, C530N, C530D, C530S, C530Q, C530E, C530A, C530H, C5301, C530L, C530K, C530M, C530F, C530P, C530T, C530W, C530Y, or C530V;
iv. D482L, D482R, D482N, D482C, D482Q, D482E, D482G, D482H, D4821, D482L, D482K, D482M, D482F, D482A, D482S, D482T, D482W, D482Y, or D482V;
v. A248L, A248R, A248N, A248C, A248Q, A248E, A248G, A248H, A2481, A248L, A248K, A248M, A248F, A248S, A248T, A248W, A248Y, or A248V
vi. ΔP427; or ΔW535; or ΔC530; or ΔD482; or ΔA248 (SEQ ID NO:123); and
vii. combinations thereof, such as:
   1. double mutants W535F/D482N (SEQ ID NO:124), P427L/W535F (SEQ ID NO:125), and P427L/C530G (SEQ ID NO:126), P427L/A248L, P427L/D482L, W535F/C530G, W535F/A248L, W535F/D482L, C530G/A248L, and A248L/D482L; and
   2. triple mutants P427L/C530G/A248L, P427L/C530G/D482L, P427L/A248L/D482L, P427L/C530G/W535F (SEQ ID NO:127), W535F/C530G/A248L, W535F/C530G/D482L, W535F/A248L/D482L, and C530G/A248L/D482L

Mutant Spy0167 proteins also include fusion polypeptides which comprise a mutant Spy0167 protein as disclosed above and another GAS antigen. GAS antigens are disclosed, e.g., in WO 02/34771 and include, but are not limited to, GAS39 (Spy0266; gi13621542), GAS40 (Spy0269; discussed below), GAS42 (Spy0287; gi13621559), GAS45 (M5005_Spy0249; gi71910063), GAS57 (Spy0416; discussed below), GAS58 (Spy0430; gi13621663), GAS67 (Spy0714; gi13621898), GAS68 (Spy0163; gi13621456), GAS84 (SPy1274; gi13622398), GAS88 (Spy1361; gi13622470), GAS 89 (Spy1390; gi13622493), GAS95 (SPy1733; gi13622787), GAS98 (Spy1882; gi13622916), GAS99 (Spy1979; gi13622993), GAS100 (Spy2000; gi13623012), GAS102 (Spy2016, gi13623025), GAS117 (Spy0448; gi13621679), GAS130 (Spy0591; gi13621794), GAS137 (Spy0652; gi13621842), GAS146 (Spy0763; gi13621942), GAS159 (Spy1105; gi13622244), GAS179 (Spy1718, gi13622773), GAS193 (Spy2025; gi3623029), GAS195 (Spy2043; gi13623043), GAS202 (Spy1309; gi13622431), GAS217 (Spy0925, gi1362208), GAS236 (Spy1126; gi13622264), GAS277 (Spy1939; gi13622962), GAS290 (SPy1959; gi13622978), GAS290 (SPy1959; gi13622978), GAS294 (Spy1173; gi13622306), GAS309 (Spy0124; gi13621426), GAS366 (Spy1525; gi13622612), GAS372 (Spy1625; gi13622698), GAS384 (Spy1874; gi13622908), GAS389 (Spy1981; gi13622996), GAS504 (Spy1751; gi13622806), GAS509 (Spy1618; gi13622692), GAS511 (Spy1743; gi13622798), GAS527 (Spy1204; gi3622332), GAS529 (Spy1280; gi3622403), GAS533 (Spy1877; gi13622912), GAS561 (Spy1134; gi13622269), GAS613 (Spy01673; gi13622736), and GAS681 (spyl152; gi1362228), as well as other antigens listed in Tables A-D, below. The gi numbers for these antigens are for the M1 strain, where available, but it will be appreciated that equivalent proteins from other M strains may also be used.

Preferred Spy0167 antigens according to the invention are immunogenic but not toxic. “Non-toxic” as used herein means that the Spy0167 antigen cannot bind to cholesterol or cannot form oligomers and, more in general, does not promote lysis of cholesterol-containing membranes. An Spy0167 protein can be rendered non-toxic, for example, by deleting at least the single cysteine residue, located in a highly conserved region in the C-terminal section of Spy0167 that can be used as a signature pattern for thiol-activated cytolysins.

Compositions of the invention also can comprise equivalents of Spy0167 mutants which are single polypeptides, which have at least 50% less hemolytic activity than wild-type Spy0167 (e.g., 50, 55, 60, 65, 70, 75, 80, 85, 90, 95, 96, 97, 98, 99, or 100%) relative to wild-type Spy0167 as determined by a hemolytic assay, which are immunogenic, and which preferably confer protection against GAS lethal challenge in a mouse model. Such equivalents may include mutant Spy0167 antigens with amino acid deletions, insertions, and/or substitutions at positions other than P427, W535, C530, A248, and D482, including deletions of up to about 40 amino acids at the N or C terminus (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids).

### Spy0269

GAS40, also known as “Spy0269” (M1), “SpyM3_{—}0197” (M3), “SpyM18_{—}0256” (M18) and “prgA,” is described, e.g., in WO 02/34771 and in WO 2005/032582. Amino acid sequences of wild-type Spy0269 are provided in SEQ ID NOS:50-106 and 128-130. Spy0269 antigens are particularly useful in compositions of the invention because Spy0269 proteins are highly conserved both in many M types and in multiple strains of these M types (see WO 2006/042027). Spy0269 consistently provides protection in the animal model of systemic immunization and challenge and induction of bactericidal antibodies.

A Spy0269 protein typically contains a leader peptide sequence (e.g., amino acids 1-26 of SEQ ID NO:50), a first coiled-coil region (e.g., amino acids 58-261 of SEQ ID NO:50), a second coiled coil region (e.g., amino acids 556-733 of SEQ ID NO:50), a leucine zipper region (e.g., amino acids 673-701 of SEQ ID NO:50) and a transmembrane region (e.g., amino acids 855-866 of SEQ ID NO:50). In some embodiments the leader sequence is removed (e.g., SEQ ID NO:177).

Compositions of the invention also can comprise equivalents of Spy0269 which are single polypeptides, which are immunogenic, and which preferably confer protection against GAS lethal challenge in a mouse model (e.g., Examples 4, 7, 8).

### Spy0416 and Immunogenic Mutants Thereof

Spy0416 (M1) is also referred to as “GAS57,” ‘SpyM3_{—}0298’ (M3), ‘SpyM18_{—}0464’(M18), and ‘prtS.’ Spy0416 has been identified as a putative cell envelope proteinase. See WO 02/34771 and US 2006/0258849. There are 49 Spy0416 sequences from 17 different M types (1, 2, 3, 4, 6, 11, 12, 18, 22, 23, 28, 44/61, 68, 75, 77, 89, 94); according to the Centers for Disease Control, the 17 different M types account for over 95% of pharyngitis cases and about 68% of the invasive GAS isolates in the United States. Amino acid sequences of wild-type Spy0416 antigens from various M types are set forth in the sequence listing as SEQ ID NOS:1-49. Compositions of the invention also can comprise equivalents of Spy0416 which are single polypeptides, which are immunogenic, and which preferably confer protection against GAS lethal challenge in a mouse model.

Mutant Spy0416 antigens according to the invention have a proteolytic activity against interleukin 8 (IL-8) which is reduced by at least 50% (e.g., 50, 55, 60, 65, 70, 75, 80, 85, 90, 95, 96, 97, 98, 99, or 100%) relative to wild-type Spy0416 as detected by either SDS-PAGE or ELISA (see Example 3), but are immunogenic, e.g., they confer protection against GAS lethal challenge in a mouse model. Preferably, a mutant Spy0416 of the invention also does not cleave other human cytokines, such as CXCL1/GROα (e.g., SEQ ID NO:131), CXCL2/GROβ (e.g., SEQ ID NO:132), CXCL3/GROγ (e.g., SEQ ID NO:133), CXCL4 (e.g., SEQ ID NO:134), CXCL12/SDF-1α (e.g., SEQ ID NO:135), CXCL12/SDF-1β (e.g., SEQ ID NO:136), CXCL12/SDF-1γ (e.g., SEQ ID NO:137), CXCL5/ENA78 (e.g., SEQ ID NO:138), CXCL6/GCP-2 (e.g., SEQ ID NO:139), CXCL7/NAP-2 (e.g., SEQ ID NO:140), CXCL9/MIG (e.g., SEQ ID NO:141), CXCL10/IP10 (e.g., SEQ ID NO:142), CXCL11 (e.g., SEQ ID NO:143), CXCL13 (e.g., SEQ ID NO:144), CXCL14 (e.g., SEQ ID NO:145), and CXCL16 (e.g., SEQ ID NO:146).

Spy0416 mutants useful in the invention include those with at an amino acid alteration (i.e., a substitution, deletion, or insertion) at one or more of amino acids D151, H279, or S617, numbered according to the wild-type Spy0416 sequence shown in SEQ ID NO:1, including single, double, or triple amino acid alterations (“single mutants,” “double mutants,” “triple mutants”) at positions D151, H279, and/or S617. Thus, Spy0416 mutants can comprise the following:
i. D151A (SEQ ID NO:147), D151R, 151N, D151C, D151Q, D151E, D151G, D151H, D151I, D151L, D151K, D151M, D151F, D151P, D1515, D151T, D151W, D151Y, or D151V;
ii. H279A, H279R, H279N, H279D, H279C, H279Q, H279E, H279G, H279I, H279L, H279K, H279M, H279F, H279P, H279S, H279T, H279W, H279Y, or H279V;
iii. S617A (SEQ ID NO:148), S617R, S617N, S617D, S617C, S617Q, S617E, S617G, S617H, S617I, S617L, S617K, S617M, S617F, S617P, S617T, S617W, S617Y, or S617V;
iv. ΔD151; or ΔH279; or ΔS617; and
v. combinations thereof, such as D151A/S617A (SEQ ID NO:149, SEQ ID NO:198).

Spy0416 mutant antigens of the invention also include fusion polypeptides which comprise a Spy0416 mutant antigen as disclosed above and another GAS antigen. GAS antigens are disclosed, e.g., in WO 02/34771 and include, but are not limited to, all or a portion of Spy0019 (GAS5; gi15675086), Spy0163 (GAS23; gi15675077), Spy0167 (GAS25, discussed above), Spy0266 (GAS39; gi13621542), Spy0269 (GAS40, discussed above), Spy0287 (GAS42; gi13621559), M5005_Spy0249 (GAS45; gi71910063), Spy0385 (GAS56; gi15675097), Spy0430 (GAS58; gi13621663), Spy0714 (GAS67; gi13621898), Spy0163 (GAS68; gi13621456), Spy1274 (GAS84; gi13622398), Spy1361 (GAS88; gi13622470), Spy1390 (GAS89; gi13622493), Spy1733 (GAS95; 13622787), Spy1882 (GAS98; gi13622916), Spy1979 (GAS99; gi13622993), Spy2000 (GAS100; gi13623012), Spy2016 (GAS102; gi15675798), Spy0448 (GAS117; gi13621679), Spy0591 (GAS130; gi13621794), Spy0652 (GAS137; gi13621842), Spy0763 (GAS146; gi15674811), Spy1105 (GAS159; gi13622244), Spy1718 (GAS179, gi13622773), Spy2025 (GAS193; gi3623029), Spy2043 (GAS195; gi15675815), Spy1309 (GAS202; gi13622431), Spy0925 (GAS217; gi1362208), Spy1126 (GAS236; gi13622264), Spy1939 (GAS277; gi13622962), Spy1959 (GAS290; gi13622978), Spy1173 (GAS294; gi13622306), Spy0124 (GAS309; gi13621426), Spy1525 (GAS366; gi13622612), Spy1625 (GAS372; gi13622698), Spy1874 (GAS384; gi13622908), Spy1981 (GAS389; gi13622996), Spy1751 (GAS504; gi13622806), Spy1618 (GAS509; gi13622692), Spy1743 (GAS511; gi13622798), Spy1204 (GAS527; gi3622332), Spy1280 (GAS529; gi3622403), Spy1877 (GAS533; gi13622912), Spy1134 (GAS561; gi13622269), Spy01673 (GAS613; gi13622736), Spy1152 (GAS681; gi1362228), or other antigens disclosed in Tables A-D below. The gi numbers for these antigens are for the M1 strain, where available, but it will be appreciated that equivalent proteins from other M strains may also be used.

The invention also includes equivalents of Spy0416 mutants which are single polypeptides, which do not cleave IL-8 as determined by SDS-PAGE or ELISA, which are immunogenic, and which preferably confer protection against GAS lethal challenge in a mouse model (e.g., Examples 4, 7, 8). Such equivalents may include mutant Spy0416 antigens with amino acid deletions, insertions, and/or substitutions at positions other than D151, H279, or S617, including deletions of up to about 40 amino acids at the N or C terminus (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids).

### Other GAS Antigens

One or more other GAS antigens can be included in compositions of the invention. GAS antigens are disclosed, for example, in WO 02/34771. Useful GAS antigens include all or portions of Spy0737, Spy0019, Spy0163, Spy0266, Spy0287 Spy0249, Spy0385, Spy0430, Spy0714, Spy0163, Spy1274, Spy1361, Spy1390, Spy1733, Spy1882, Spy1979, Spy2000, Spy2016, Spy0448, Spy0591, Spy0652, Spy0763, Spy1105, Spy1718, Spy2025, Spy2043, Spy1309, Spy0925, Spy1126, Spy1939, Spy1959, Spy1173, Spy0124, Spy1525, Spy1625, Spy1874, Spy1981, Spy1751, Spy1618, Spy1743, Spy1204, Spy1280, Spy1877, Spy1134, and Spy01673. Compositions of the invention also can comprise equivalents of these GAS antigens which are single polypeptides, which are immunogenic, and which preferably confer protection against GAS lethal challenge in a mouse model (e.g., Examples 4, 7, 8). For example, each of Spy0763 (GAS146) and Spy1134 (GAS561) protects mice against challenge with S. pyogenes M1 3348 (70% survival compared with 20% survival of the negative controls; n=20). See also Tables A-D, below.

TABLE A

| | | | | | in sequenced strains | | | |
|---|---|---|---|---|---|---|---|---|
| | SEQ | | | | genetic | average % | Serotype distribution: | |
| ANTIGEN | ID NO: | Surfome | Secretome | FACS | distribution | identity | present in | missing in |
| spy0019 | 178 | 2/4 | 4/4 | pos. in 2/4 | 12/12 | >90% | | |
| spy0163 | 179 | no | 2/4 | pos. in 1/4 | 12/12 | >90% | | |
| spy0385 | 180 | no | no | not tested | 12/12 | >90% | | |
| spy0714 | 181 | no | no | not tested | 12/12 | >90% | | |
| spy0737 | 182 | no | no | not tested | 6/12 | 70% | M1, M4, M12, M28, | missing in |
| | | | | | | | M49 | M2, M3, |
| | | | | | | | | M5, M6, |
| | | | | | | | | M18 |
| spy1274 | 183 | no | no | not tested | 11/12 | >90% | M1, M2, M4, M5, | missing in |
| | | | | | | | M12, M28, M49 | M6 |
| spy1361 | 184 | no | no | not tested | 12/12 | >90% | | |
| spy1390 | 185 | 1/4 | 2/4 | pos. in 2/4 | 12/12 | >90% | | |
| spy1733 | 186 | no | no | pos. in 2/4 | 12/12 | >90% | | |
| spy1882 | 187 | 4/4 | 2/4 | pos. in 2/4 | 12/12 | >90% | | |
| spy1979 | 188 | no | 1/4 | pos. in 2/4 | 12/12 | >90% | | |
| spy2000 | 189 | no | 1/4 | pos. in 1/4 | 12/12 | >90% | | |
| spy2016 | 190 | no | no | not tested | 4/12, variants | >90% | M1, M12 | missing in |
| | | | | | M1 and M12 | within | | all the |
| | | | | | | variants | | others |
| spy0591 | 191 | no | no | pos. in 2/4 | 12/12 | >90% | | |
| spy1105 | 192 | no | no | not tested | 12/12 | >90% | | |
| spy1718 | 193 | 2/4 | no | pos. in 2/4 | 12/12 | >90% | Variant 1: M1, M2, | |
| | | | | | | within | M3, M5, M6, M12, | |
| | | | | | | variants | M18, Variant 2: M4, | |
| | | | | | | | M28, M49 | |
| spy2025 | 194 | no | no | not tested | 12/12 | >90% | | |
| spy2043 | 195 | no | 3/4 | pos. in 3/4 | 12/12 | >90% | | |
| spy1939 | 196 | 1/4 | 3/4 | pos. in 2/4 | 12/12 | >90% | | |
| spy1625 | 197 | no | no | not tested | 12/12 | >90% | | |

TABLE B

| | | | | | | | | | | | | | ALL | ALL |
|---|---|---|---|---|---|---|---|---|---|---|---|---|---|---|
| | | | | | | | | | | | | | STRAINS | STRAINS |
| ANTI- | 3348 M1 | | | EM5 M12 | | | 2721 M3 | | | 2071 M23 | | | SURF | SECR |
| GEN | FACS | SURF | SECR | FACS | SURF | SECR | FACS | SURF | SECR | FACS | SURF | SECR | FREQ | FREQ |
| | 322 | | Y | 268 | Y | Y | 13 | | Y | 31 | Y | Y | | ALL |
| spy0019 | 122 | | Y | 26 | | Y | 33 | | | 58 | | | | 2/4 |
| spy0163 | | | | | | | | | | | | | | |
| spy0385 | | | | | | | | | | | | | | |
| spy0714 | | | | | | | | | | | | | | |
| spy0737 | | | | | | | | | | | | | | |
| spy1274 | | | | | | | | | | | | | | |
| spy1361 | 120 | Y | Y | 254 | | | 7 | | Y | 92 | | | | 2/4 |
| spy1390 | 62 | | | 209 | | | 6 | | | 115 | | | | |
| spy1733 | 335 | Y | Y | 146 | Y | | 4 | Y | | 40 | Y | Y | 5/16 | 2/4 |
| spy1882 | 186 | | Y | 188 | | | 9 | | | 59 | | | | 1/4 |
| spy1979 | 163 | | Y | 80 | | | 15 | | | 22 | | | | 1/4 |
| spy2016 | 186 | | | 119 | | | 43 | | | 48 | | | | |
| spy0591 | | | | | | | | | | | | | | |
| spy1105 | 31 | Y | | 257 | | | 3 | Y | | 141 | | | | |
| spy1718 | | | | | | | | | | | | | | |
| spy2025 | 332 | | Y | 349 | | | 26 | | Y | 359 | | Y | | 3/4 |
| spy2043 | 225 | Y | Y | 203 | | | 37 | | Y | 71 | | Y | | 3/4 |
| spy1939 | | | | | | | | | | | | | | |
| spy1625 | | | | | | | | | | | | | | |

TABLE C

Protection

| | | M1 | M12 | M23 |
|---|---|---|---|---|
| SEQ ID NO: | ANTIGEN | + | + | − |
| 178 | spy0019 | + | nd | nd |
| 179 | spy0163 | | | |
| 180 | spy0385 | | | |
| 181 | spy0714 | | | |
| 182 | spy0737 | | | |
| 183 | spy1274 | | | |
| 184 | spy1361 | + | − | nd |
| 185 | spy1390 | nd | + | nd |
| 186 | spy1733 | + | − | − |
| 187 | spy1882 | + | − | + |
| 188 | spy1979 | − | + | − |
| 189 | spy2000 | | | |
| 190 | spy2016 | + | − | + |
| 191 | spy0591 | | | |
| 192 | spy1105 | nd | − | + |
| 193 | spy1718 | | | |
| 194 | spy2025 | + | + | − |
| 195 | spy2043 | − | − | nd |
| 196 | spy1939 | | | |
| 197 | spy1625 | | | |

TABLE D

| | | FACS details | | | |
|---|---|---|---|---|---|
| | SEQ ID | 3348 M1 | EM5 M12 | 2721 M3 | 2071 M23 |
| ANTIGEN | NO: | 322 | 268 | 13 | 31 |
| spy0019 | 178 | 122 | 26 | 33 | 58 |
| spy0163 | 179 | | | | |
| spy0385 | 180 | | | | |
| spy0714 | 181 | | | | |
| spy0737 | 182 | | | | |
| spy1274 | 183 | | | | |
| spy1361 | 184 | 120 | 254 | 7 | 92 |
| spy1390 | 185 | 62 | 209 | 6 | 115 |
| spy1733 | 186 | 335 | 146 | 4 | 40 |
| spy1882 | 187 | 186 | 188 | 9 | 59 |
| spy1979 | 188 | 163 | 80 | 15 | 22 |
| spy2000 | 189 | | | | |
| spy2016 | 190 | 186 | 119 | 43 | 48 |
| spy0591 | 191 | | | | |
| spy1105 | 192 | 31 | 257 | 3 | 141 |
| spy1718 | 193 | | | | |
| spy2025 | 194 | 332 | 349 | 26 | 359 |
| spy2043 | 195 | 225 | 203 | 37 | 71 |
| spy1939 | 196 | | | | |
| spy1625 | 197 | | | | |

### Fragments

The length of fragments of the wild-type or mutant GAS proteins described above may vary depending on the amino acid sequence of the specific GAS antigen or mutant thereof, but the fragment is preferably at least seven consecutive amino acids, e.g., 8, 10, 12, 14, 16, 18, 20, 25, 30, 35, 40, 50, 60, 70, 80, 90, 100, 150, 200 or more, up to one amino acid less than a full-length wild-type or mutant GAS protein. Preferably the fragment is immunogenic and comprises one or more epitopes from the sequence. Other preferred fragments include (1) the N-terminal signal peptides of each identified GAS protein, (2) the identified GAS protein without their N-terminal signal peptides, and (3) each identified GAS protein in which up to 10 amino acid residues (e.g., 1, 2, 3, 4, 5, 6, 7, 8, 9, 10, 15, 20, 25 or more) are deleted from the N-terminus and/or the C-terminus (for example, the N-terminal amino acid residue may be deleted). Other fragments omit one or more domains of the protein (e.g., omission of a signal peptide, a cytoplasmic domain, a transmembrane domain, or an extracellular domain). In some embodiments the fragment is amino acids 33-324 of Spy0269.

### GAS Polysaccharide Antigen

GAS polysaccharide (PS) is a cell-wall polysaccharide present in all GAS strains.

Antibody titers to PS correlate inversely with disease and colonization in children. In some embodiments compositions of the invention comprise a GAS polysaccharide antigen. S. pyogenes GAS carbohydrate typically features a branched structure with an L-rhamnopyranose (Rhap) backbone consisting of alternating alpha-(1→2) and alpha-(1→3) links and D-N-acetylglucosamine (GlcpNAc) residues beta-(1→3)-connected to alternating rhamnose rings (Kreis et al., Int. J. Biol. Macromol. 17, 117-30, 1995). GAS polysaccharide antigens useful in compositions of the invention have the formula:

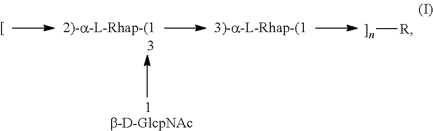

wherein R is a terminal reducing L-Rhamnose or D-GlcpNAc and n is a number from about 3 to about 30.

The GAS polysaccharide antigen used according to the invention may be a substantially full-length GAS carbohydrate, as found in nature, or it may be shorter than the natural length. Full-length polysaccharides may be depolymerized to give shorter fragments for use with the invention, e.g., by hydrolysis in mild acid, by heating, by sizing chromatography, etc. However, it is preferred to use saccharides of substantially full-length. In particular, it is preferred to use saccharides with a molecular weight of about 10 kDa. Molecular masses can be measured by gel filtration relative to dextran standards.

The saccharide may be chemically modified relative to the GAS carbohydrate as found in nature. For example, the saccharide may be de N acetylated (partially or fully), N propionated (partially or fully), etc. The effect of de-acetylation etc., for example on immunogenicity, can be assessed by routine assays.

In some embodiments the GAS polysaccharide antigen is conjugated to a carrier, such as the mutated diphtheria toxin CRM197 (and other carriers described below. As described in the Examples, below, antibodies to PS conjugated with CRM197 (“GC”) induce GAS opsonophagocytic killing.

### Production of GAS Protein Antigens

Recombinant Production

The redundancy of the genetic code is well-known. Thus, any nucleic acid molecule (polynucleotide) which encodes one of the GAS antigens described herein can be used to produce that protein recombinantly. Nucleic acid molecules encoding wild-type GAS antigens also can be isolated from the appropriate S. pyogenes bacterium using standard nucleic acid purification techniques or can be synthesized using an amplification technique, such as the polymerase chain reaction (PCR), or by using an automatic synthesizer. See Caruthers et al., Nucl. Acids Res. Symp. Ser. 215, 223, 1980; Horn et al., Nucl. Acids Res. Symp. Ser. 225, 232, 1980; Hunkapiller et al., Nature 310, 105-11, 1984; Grantham et al., Nucleic Acids Res. 9, r43-r74, 1981.

cDNA molecules can be made with standard molecular biology techniques, using mRNA as a template. cDNA molecules can thereafter be replicated using molecular biology techniques well known in the art. An amplification technique, such as PCR, can be used to obtain additional copies of polynucleotides of the invention, using either genomic DNA or cDNA as a template.

If desired, polynucleotides can be engineered using methods generally known in the art to alter antigen-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site directed mutagenesis can be used to insert new restriction sites, alter glycosylation patterns, change codon preference, produce splice variants, introduce mutations, and so forth.

Sequence modifications, such as the addition of a purification tag sequence or codon optimization, can be used to facilitate expression. For example, the N-terminal leader sequence may be replaced with a sequence encoding for a tag protein such as polyhistidine (“HIS”) or glutathione S-transferase (“GST”). Such tag proteins may be used to facilitate purification, detection, and stability of the expressed protein. Codons preferred by a particular prokaryotic or eukaryotic host can be selected to increase the rate of protein expression or to produce an RNA transcript having desirable properties, such as a half life which is longer than that of a transcript generated from the naturally occurring sequence. These methods are well known in the art and are further described in WO05/032582.

Expression Vectors

A nucleic acid molecule which encodes a GAS antigen for use in the invention can be inserted into an expression vector which contains the necessary elements for the transcription and translation of the inserted coding sequence. Methods which are well known to those skilled in the art can be used to construct expression vectors containing coding sequences and appropriate transcriptional and translational control elements. These methods include in vitro recombinant DNA techniques, synthetic techniques, and in vivo genetic recombination.

Host Cells

Host cells for producing GAS antigens can be prokaryotic or eukaryotic. E. coli is a preferred host cell, but other suitable hosts include Lactococcus lactis, Lactococcus cremoris, Bacillus subtilis, Vibrio cholerae, Salmonella typhi, Salmonella typhimurium, Neisseria lactamica, Neisseria cinerea, Mycobacteria (e.g., M. tuberculosis), yeasts, baculovirus, mammalian cells, etc.

A host cell strain can be chosen for its ability to modulate the expression of the inserted sequences or to process the expressed polypeptide in the desired fashion. Such modifications of the polypeptide include, but are not limited to, acetylation, carboxylation, glycosylation, phosphorylation, lipidation, and acylation. Post translational processing which cleaves a “prepro” form of the polypeptide also can be used to facilitate correct insertion, folding and/or function. Different host cells which have specific cellular machinery and characteristic mechanisms for post translational activities are available from the American Type Culture Collection (ATCC; 10801 University Boulevard, Manassas, Va. 20110-2209) and can be chosen to ensure the correct modification and processing of a foreign protein. See WO 01/98340.

Expression constructs can be introduced into host cells using well-established techniques which include, but are not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun” methods, and DEAE- or calcium phosphate-mediated transfection.

Host cells transformed with expression vectors can be cultured under conditions suitable for the expression and recovery of the protein from cell culture. The protein produced by a transformed cell can be secreted or contained intracellularly depending on the nucleotide sequence and/or the expression vector used. Those of skill in the art understand that expression vectors can be designed to contain signal sequences which direct secretion of soluble antigens through a prokaryotic or eukaryotic cell membrane.

Purification

Signal export sequences can be included in a recombinantly produced GAS antigen so that the antigen can be purified from cell culture medium using known methods. Alternatively, recombinantly produced GAS antigens can be isolated from engineered host cells and separated from other components in the cell, such as proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified GAS antigens is at least 80% pure; preferably, the preparations are 90%, 95%, or 99% pure. Purity of the preparations can be assessed by any means known in the art, such as SDS-polyacrylamide gel electrophoresis or RP-HPLC analysis. Where appropriate, mutant Spy0167 proteins can be solubilized, for example, with urea.

Chemical Synthesis

GAS antigens can be synthesized, for example, using solid phase techniques. See, e.g., Merrifield, J. Am. Chem. Soc. 85, 2149 54, 1963; Roberge et al., Science 269, 202 04, 1995. Protein synthesis can be performed using manual techniques or by automation. Automated synthesis can be achieved, for example, using Applied Biosystems 431A Peptide Synthesizer (Perkin Elmer). Optionally, fragments of GAS antigens can be separately synthesized and combined using chemical methods to produce a full-length molecule.

### Antibodies

Some compositions of the invention comprise combinations of antibodies which specifically bind to GAS antigens described herein. An antibody “specifically binds” to a GAS antigen if it provides a detection signal at least 5-, 10-, or 20-fold higher than a detection signal provided with a different protein when used in an immunochemical assay. Preferably, antibodies that specifically bind to a GAS antigen do not detect other proteins in immunochemical assays and can immunoprecipitate the GAS antigen from solution.

The term “antibody” includes intact immunoglobulin molecules, as well as fragments thereof which are capable of binding an antigen. These include hybrid (chimeric) antibody molecules (e.g., Winter et al., Nature 349, 293-99, 1991; U.S. Pat. No. 4,816,567); F(ab′)2 and F(ab) fragments and Fv molecules; non-covalent heterodimers (e.g., Inbar et al., Proc. Natl. Acad. Sci. U.S.A. 69, 2659-62, 1972; Ehrlich et al., Biochem 19, 4091-96, 1980); single-chain Fv molecules (sFv) (e.g., Huston et al., Proc. Natl. Acad. Sci. U.S.A. 85, 5897-83, 1988); dimeric and trimeric antibody fragment constructs; minibodies (e.g., Pack et al., Biochem 31, 1579-84, 1992; Cumber et al., J. Immunology 149B, 120-26, 1992); humanized antibody molecules (e.g., Riechmann et al., Nature 332, 323-27, 1988; Verhoeyan et al., Science 239, 1534-36, 1988; and U.K. Patent Publication No. GB 2,276,169, published 21 Sep. 1994); and any functional fragments obtained from such molecules, as well as antibodies obtained through non-conventional processes such as phage display. Preferably, the antibodies are monoclonal antibodies. Methods of obtaining monoclonal antibodies are well known in the art.

Typically, at least 6, 7, 8, 10, or 12 contiguous amino acids are required to form an epitope. However, epitopes which involve non-contiguous amino acids may require more, e.g., at least 15, 25, or 50 amino acids. Various immunoassays (e.g., Western blots, ELISAs, radioimmunoassays, immunohistochemical assays, immunoprecipitations, or other immunochemical assays known in the art) can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen. A preparation of antibodies which specifically bind to a GAS antigen typically provides a detection signal at least 5-, 10-, or 20-fold higher than a detection signal provided with other proteins when used in an immunochemical assay and does not provide a detectable signal if contacted with an “irrelevant” protein. Preferably, the antibodies do not detect other proteins in immunochemical assays and can immunoprecipitate the particular antigen from solution.

Antibodies which specifically bind to wild-type Spy0167 substantially reduce (e.g., by at least 50%, 60%, 70%, 80%, 90%, 95%, 97%, 98%, or 99%) or eliminate its hemolytic activity. Some antibodies also specifically bind to the mutant Spy0167 proteins described above.

Antibodies which specifically bind to wild-type Spy0416 substantially reduce (e.g., by at least 50%) or eliminate the ability of Spy0416 to cleave IL-8 (Example 5). Antibodies may reduce the ability of Spy0416 to cleave IL-8 by at least 50%, 60%, 70%, 80%, 90%, 95%, 97%, 98%, or 99%. Some antibodies also specifically bind to the mutant Spy0416 proteins described above. Preferred antibodies also reduce or eliminate the ability of Spy0416 to cleave other substrates such as homologs of IL-8 (e.g., CXCL1/GROα, CXCL2/GROβ, CXCL3/GROγ, CXCL4, CXCL12/SDF-1α, CXCL12/SDF-1β, CXCL12/SDF-1γ, CXCL5/ENA 78, CXCL6/GCP-2, CXCL7/NAP-2, CXCL9/MIG, CXCL10/IP10, CXCL11, CXCL13, CXCL14, and CXCL16. Some antibodies block the progression of necrotic lesions in animals immunized with wild-type or mutant Spy0416 recombinant antigen and challenged with GAS.

Antibodies which specifically bind to Spy0269 substantially reduce (e.g., by at least 50%, 60%, 70%, 80%, 90%, 95%, 97%, 98%, or 99%) or eliminate binding of Spy0269 to epithelial cells as measured by the cell binding assay described in Example 25.

Generation of Antibodies

GAS antigens can be used to immunize a mammal, such as a mouse, rat, rabbit, guinea pig, monkey, or human, to produce polyclonal antibodies. If desired, an antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to an antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495 497, 1985; Kozbor et al., J. Immunol. Methods 81, 3142, 1985; Cote et al., Proc. Natl. Acad. Sci. 80, 2026 2030, 1983; Cole et al., Mol. Cell. Biol. 62, 109 120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 68516855, 1984; Neuberger et al., Nature 312, 604 608, 1984; Takeda et al., Nature 314, 452 454, 1985). Monoclonal and other antibodies also can be “humanized” to prevent or reduce the risk of a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120 23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J. Cancer 61, 497-501, 1995; Nicholls et al., J. Immunol. Meth. 165, 81-91, 1993).

Antibodies which specifically bind to a particular antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

### Pharmaceutical Compositions

The invention also provides compositions for use as medicaments (e.g., as immunogenic compositions or vaccines). Compositions of the invention are useful for preventing S. pyogenes infection, reducing the risk of S. pyogenes infection, and/or treating disease caused as a result of S. pyogenes infection, such as bacteremia, meningitis, puerperal fever, scarlet fever, erysipelas, pharyngitis, impetigo, necrotizing fasciitis, myositis or toxic shock syndrome.

Compositions containing GAS antigens are preferably immunogenic compositions, and are more preferably vaccine compositions. The pH of such compositions preferably is between 6 and 8, preferably about 7. The pH can be maintained by the use of a buffer. The composition can be sterile and/or pyrogen free. The composition can be isotonic with respect to humans.

Vaccines according to the invention may be used either prophylactically or therapeutically, but will typically be prophylactic. Accordingly, the invention includes a method for the therapeutic or prophylactic treatment of a Streptococcus pyogenes infection. The animal is preferably a mammal, most preferably a human. The methods involve administering to the animal a therapeutic or prophylactic amount of the immunogenic compositions of the invention. The invention also provides the immunogenic compositions of the invention for treating, reducing the risk or, and/or preventing a S. pyogenes infection.

Some compositions of the invention comprise two different GAS antigens, as described above. Other compositions of the invention comprise at least one nucleic acid molecule which encodes the two different antigens. See, e.g., Robinson & Tones (1997) Seminars in Immunology 9:271-283; Donnelly et al. (1997) Ann. Rev Immunol 15:617-648; Scott-Taylor & Dalgleish (2000) Expert Opin Investig Drugs 9:471-480; Apostolopoulos & Plebanski (2000) Curr Opin Mol Ther 2:441-447; Ilan (1999) Curr Opin Mol Ther 1:116-120; Dubensky et al. (2000) Mol Med 6:723-732; Robinson & Pertmer (2000) Adv Virus Res 55:1-74; Donnelly et al. (2000) Am J Respir Crit. Care Med 162(4 Pt 2):S190-193; Davis (1999) Mt. Sinai J. Med. 66:84-90. Typically the nucleic acid molecule is a DNA molecule, e.g., in the form of a plasmid.

Other compositions of the invention, which are useful therapeutically, comprise two different antibodies, each of which specifically binds to one of the two different GAS antigens.

In some embodiments, compositions of the invention can include one or more additional active agents. Such agents include, but are not limited to, (a) a polypeptide antigen which is useful in a pediatric vaccine, (b) a polypeptide antigen which is useful in a vaccine for elderly or immunocompromised individuals, (c) a nucleic acid molecule encoding (a) or (b), and (d) an antibody which specifically binds to (a) or (b).

Additional Antigens

Compositions of the invention may be administered in conjunction with one or more additional antigens for use in therapeutic or prophylactic methods of the present invention. Suitable antigens include those listed below. Additionally, the compositions of the present invention may be used to treat, reduce the risk of, or prevent infections caused by any of the below-listed pathogens. In addition to combination with the antigens described below, the compositions of the invention may also be combined with an adjuvant as described herein.

Additional antigens for use with the invention include, but are not limited to, one or more of the following antigens set forth below, or antigens derived from one or more of the pathogens set forth below:

A. Bacterial Antigens

Bacterial antigens suitable for use in the invention include proteins, polysaccharides, lipopolysaccharides, and outer membrane vesicles which may be isolated, purified or derived from a bacteria. In addition, bacterial antigens may include bacterial lysates and inactivated bacteria formulations. Bacteria antigens may be produced by recombinant expression. Bacterial antigens preferably include epitopes which are exposed on the surface of the bacteria during at least one stage of its life cycle. Bacterial antigens are preferably conserved across multiple serotypes. Bacterial antigens include antigens derived from one or more of the bacteria set forth below as well as the specific antigens examples identified below.

Neisseria meningitides: Meningitides antigens may include proteins (such as those identified in References 1-7), saccharides (including a polysaccharide, oligosaccharide or lipopolysaccharide), or outer-membrane vesicles (References 8, 9, 10, 11) purified or derived from N. meningitides serogroup such as A, C, W135, Y, and/or B. Meningitides protein antigens may be selected from adhesions, autotransporters, toxins, Fe acquisition proteins, and membrane associated proteins (preferably integral outer membrane protein).

Streptococcus pneumoniae: Streptococcus pneumoniae antigens may include a saccharide (including a polysaccharide or an oligosaccharide) and/or protein from Streptococcus pneumoniae. Saccharide antigens may be selected from serotypes 1, 2, 3, 4, 5, 6B, 7F, 8, 9N, 9V, 10A, 11A, 12F, 14, 15B, 17F, 18C, 19A, 19F, 20, 22F, 23F, and 33F. Protein antigens may be selected from a protein identified in WO 98/18931, WO 98/18930, U.S. Pat. No. 6,699,703, U.S. Pat. No. 6,800,744, WO 97/43303, and WO 97/37026. Streptococcus pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, Sp101, Sp130, Sp125 or Sp133.

Streptococcus pyogenes (Group A Streptococcus): Group A Streptococcus antigens may include a protein identified in WO 02/34771 or WO 2005/032582 (including, but not limited to, GAS39 (Spy0266), GAS40 (Spy0269, discussed above), GAS42 (Spy0287), GAS45(M5005_Spy0249), GAS57 (Spy0416), GAS58 (Spy0430), GAS67 (Spy0714), GAS68 (Spy0163), GAS84 (SPy1274), GAS88 (Spy1361), GAS 89 (Spy1390) GAS95 (SPy1733), GAS98 (Spy1882), GAS99 (Spy1979), GAS100 (Spy2000), GAS102 (Spy2016), GAS117 (Spy0448), GAS130 (Spy0591), GAS137 (Spy0652), GAS146 (Spy0763), GAS159 (Spy1105), GAS179 (Spy1718), GAS193 (Spy2025), GAS195 (Spy2043), GAS202 (Spy1309), GAS217 (Spy0925), GAS236 (Spy1126), GAS277 (Spy1939), GAS294 (Spy1173), GAS309 (Spy0124), GAS366 (Spy1525), GAS372 (Spy1625), GAS384 (Spy1874), GAS389 (Spy1981), GAS504 (Spy1751), GAS509 (Spy1618), GAS290 (SPy1959), GAS511(Spy1743), GAS527 (Spy1204), GAS529 (Spy1280), GAS533(Spy1877), GAS561 (Spy1134), GAS613 (Spy01673), and GAS681 (spyl152), other GAS antigens described above and in Tables A-D, fusions of fragments of GAS M proteins (including those described in WO 02/094851, and Dale, Vaccine (1999) 17:193-200, and Dale, Vaccine 14(10): 944-948), fibronectin binding protein (Sfb1), Streptococcal heme-associated protein (Shp), and Streptolysin S (SagA).

Moraxella catarrhalis: Moraxella antigens include antigens identified in WO 02/18595 and WO 99/58562, outer membrane protein antigens (HMW-OMP), C-antigen, and/or LPS.

Bordetella pertussis: Pertussis antigens include petussis holotoxin (PT) and filamentous hemagglutinin (FHA) from B. pertussis, optionally also combination with pertactin and/or agglutinogens 2 and 3 antigen.

Staphylococcus aureus: Staphylococcus aureus antigens include S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin).

Staphylococcus epidermis: S. epidermidis antigens include slime-associated antigen (SAA).

Clostridium tetani (Tetanus): Tetanus antigens include tetanus toxoid (TT), preferably used as a carrier protein in conjunction/conjugated with the compositions of the present invention.

Cornynebacterium diphtheriae (Diphtheria): Diphtheria antigens include diphtheria toxin, preferably detoxified, such as CRM197. Additionally antigens capable of modulating, inhibiting or associated with ADP ribosylation are contemplated for combination/co-administration/conjugation with the compositions of the present invention. The diphtheria toxoids may be used as carrier proteins.

Haemophilus influenzae B (Hib): Hib antigens include a Hib saccharide antigen.

Pseudomonas aeruginosa: Pseudomonas antigens include endotoxin A, Wzz protein, P. aeruginosa LPS, more particularly LPS isolated from PAO1 (O5 serotype), and/or Outer Membrane Proteins, including Outer Membrane Proteins F (OprF) (Infect Immun. 2001 May; 69(5): 3510-3515).

Legionella pneumophila. Bacterial antigens may be derived from Legionella pneumophila.

Streptococcus agalactiae (Group B Streptococcus): Group B Streptococcus antigens include a protein or saccharide antigen identified in WO 02/34771, WO 03/093306, WO 04/041157, or WO 2005/002619 (including proteins GBS 80, GBS 104, GBS 276 and GBS 322, and including saccharide antigens derived from serotypes Ia, Ib, Ia/c, II, III, IV, V, VI, VII and VIII).

Neiserria gonorrhoeae: Gonorrhoeae antigens include Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J. Infectious Disease 182, 848-55, 2000), also see e.g. WO99/24578, WO99/36544, WO99/57280, WO02/079243).

Chlamydia trachomatis: Chlamydia trachomatis antigens include antigens derived from serotypes A, B, Ba and C (agents of trachoma, a cause of blindness), serotypes L1, L2 & L3 (associated with Lymphogranuloma venereum), and serotypes, D-K. Chlamydia trachomas antigens may also include an antigen identified in WO 00/37494, WO 03/049762, WO 03/068811, or WO 05/0026f19, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761).

Treponema pallidum (Syphilis): Syphilis antigens include TmpA antigen.

Haemophilus ducreyi (causing chancroid): Ducreyi antigens include outer membrane protein (DsrA).

Enterococcus faecalis or Enterococcus faecium: Antigens include a trisaccharide repeat or other Enterococcus derived antigens provided in U.S. Pat. No. 6,756,361.

Helicobacter pylori: H. pylori antigens include Cag, Vac, Nap, HopX, HopY and/or urease antigen.

Staphylococcus saprophyticus: Antigens include the 160 kDa hemagglutinin of S. saprophyticus antigen.

Yersinia enterocolitica antigens include LPS (Infect Immun. 2002 August; 70(8): 4414).

E. coli: E. coli antigens may be derived from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC).

Bacillus anthracis (anthrax): B. anthracis antigens are optionally detoxified and may be selected from A-components (lethal factor (LF) and edema factor (EF)), both of which can share a common B-component known as protective antigen (PA).

Yersinia pestis (plague): Plague antigens include F1 capsular antigen (Infect Immun. 2003 January; 71(1)): 374-383, LPS (Infect Immun. 1999 October; 67(10): 5395), Yersinia pestis V antigen (Infect Immun. 1997 November; 65(11): 4476-4482).

Mycobacterium tuberculosis: Tuberculosis antigens include lipoproteins, LPS, BCG antigens, a fusion protein of antigen 85B (Ag85B) and/or ESAT-6 optionally formulated in cationic lipid vesicles (Infect Immun. 2004 October; 72(10): 6148), Mycobacterium tuberculosis (Mtb) isocitrate dehydrogenase associated antigens (Proc Natl Acad Sci USA. 2004 Aug. 24; 101(34): 12652), and/or MPT51 antigens (Infect Immun. 2004 July; 72(7): 3829).

Rickettsia: Antigens include outer membrane proteins, including the outer membrane protein A and/or B (OmpB) (Biochim Biophys Acta. 2004 Nov. 1; 1702(2):145), LPS, and surface protein antigen (SPA) (J. Autoimmun. 1989 June; 2 Supp1:81).

Listeria monocytogenes. Bacterial antigens may be derived from Listeria monocytogenes.

Chlamydia pneumoniae: Antigens include those identified in WO 02/02606.

Vibrio cholerae: Antigens include proteinase antigens, LPS, particularly lipopolysaccharides of Vibrio cholerae II, O1 Inaba O-specific polysaccharides, V. cholera O139, antigens of IEM108 vaccine (Infect Immun. 2003 October; 71(10):5498-504), and/or Zonula occludens toxin (Zot).

Salmonella typhi (typhoid fever): Antigens include capsular polysaccharides preferably conjugates (Vi, i.e. vax-TyVi).

Borrelia burgdorferi (Lyme disease): Antigens include lipoproteins (such as OspA, OspB, Osp C and Osp D), other surface proteins such as OspE-related proteins (Erps), decorin-binding proteins (such as DbpA), and antigenically variable VI proteins, such as antigens associated with P39 and P13 (an integral membrane protein, Infect Immun. 2001 May; 69(5): 3323-3334), V1sE Antigenic Variation Protein (J Clin Microbiol. 1999 December; 37(12): 3997).

Porphyromonas gingivalis: Antigens include P. gingivalis outer membrane protein (OMP).

Klebsiella: Antigens include an OMP, including OMP A, or a polysaccharide optionally conjugated to tetanus toxoid.

Further bacterial antigens of the invention may be capsular antigens, polysaccharide antigens or protein antigens of any of the above. Further bacterial antigens may also include an outer membrane vesicle (OMV) preparation. Additionally, antigens include live, attenuated, and/or purified versions of any of the aforementioned bacteria. The antigens of the present invention may be derived from gram-negative or gram-positive bacteria. The antigens of the present invention may be derived from aerobic or anaerobic bacteria.

Additionally, any of the above bacterial-derived saccharides (polysaccharides, LPS, LOS or oligosaccharides) can be conjugated to another agent or antigen, such as a carrier protein (for example CRM197). Such conjugation may be direct conjugation effected by reductive amination of carbonyl moieties on the saccharide to amino groups on the protein, as provided in U.S. Pat. No. 5,360,897 and Can J Biochem Cell Biol. 1984 May; 62(5):270-5. Alternatively, the saccharides can be conjugated through a linker, such as, with succinamide or other linkages provided in Bioconjugate Techniques, 1996 and CRC, Chemistry of Protein Conjugation and Cross-Linking, 1993.

B. Viral Antigens

Viral antigens suitable for use in the invention include inactivated (or killed) virus, attenuated virus, split virus formulations, purified subunit formulations, viral proteins which may be isolated, purified or derived from a virus, and Virus Like Particles (VLPs). Viral antigens may be derived from viruses propagated on cell culture or other substrate. Alternatively, viral antigens may be expressed recombinantly. Viral antigens preferably include epitopes which are exposed on the surface of the virus during at least one stage of its life cycle. Viral antigens are preferably conserved across multiple serotypes or isolates. Viral antigens include antigens derived from one or more of the viruses set forth below as well as the specific antigens examples identified below.

Orthomyxovirus: Viral antigens may be derived from an Orthomyxovirus, such as Influenza A, B and C. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase components (PB 1, PB2 and PA). Preferred antigens include HA and NA.

Influenza antigens may be derived from interpandemic (annual) flu strains. Alternatively influenza antigens may be derived from strains with the potential to cause pandemic a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).

Paramyxoviridae viruses: Viral antigens may be derived from Paramyxoviridae viruses, such as Pneumoviruses (RSV), Paramyxoviruses (PIV) and Morbilliviruses (Measles).

Pneumovirus: Viral antigens may be derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. Pneumovirus antigens may be selected from one or more of the following proteins, including surface proteins Fusion (F), Glycoprotein (G) and Small Hydrophobic protein (SH), matrix proteins M and M2, nucleocapsid proteins N, P and L and nonstructural proteins NS 1 and NS2. Preferred Pneumovirus antigens include F, G and M. See e.g., J Gen Virol. 2004 November; 85(Pt 11):3229). Pneumovirus antigens may also be formulated in or derived from chimeric viruses. For example, chimeric RSV/PIV viruses may comprise components of both RSV and PIV.

Paramyxovirus: Viral antigens may be derived from a Paramyxovirus, such as Parainfluenza virus types 1-4 (PIV), Mumps, Sendai viruses, Simian virus 5, Bovine parainfluenza virus and Newcastle disease virus. Preferably, the Paramyxovirus is PIV or Mumps. Paramyxovirus antigens may be selected from one or more of the following proteins: Hemagglutinin—Neuraminidase (HN), Fusion proteins F1 and F2, Nucleoprotein (NP), Phosphoprotein (P), Large protein (L), and Matrix protein (M). Preferred Paramyxovirus proteins include HN, F1 and F2. Paramyxovirus antigens may also be formulated in or derived from chimeric viruses. For example, chimeric RSV/PIV viruses may comprise components of both RSV and PIV. Commercially available mumps vaccines include live attenuated mumps virus, in either a monovalent form or in combination with measles and rubella vaccines (MMR).

Morbillivirus: Viral antigens may be derived from a Morbillivirus, such as Measles. Morbillivirus antigens may be selected from one or more of the following proteins: hemagglutinin (H), Glycoprotein (G), Fusion factor (F), Large protein (L), Nucleoprotein (NP), Polymerase phosphoprotein (P), and Matrix (M). Commercially available measles vaccines include live attenuated measles virus, typically in combination with mumps and rubella (MMR).

Picornavirus: Viral antigens may be derived from Picornaviruses, such as Enteroviruses, Rhinoviruses, Heparnavirus, Cardioviruses and Aphthoviruses. Antigens derived from Enteroviruses, such as Poliovirus are preferred.

Enterovirus: Viral antigens may be derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. Preferably, the Enterovirus is poliovirus. Enterovirus antigens are preferably selected from one or more of the following Capsid proteins VP1, VP2, VP3 and VP4. Commercially available polio vaccines include Inactivated Polio Vaccine (IPV) and Oral poliovirus vaccine (OPV).

Heparnavirus: Viral antigens may be derived from an Heparnavirus, such as Hepatitis A virus (HAV). Commercially available HAV vaccines include inactivated HAV vaccine.

Togavirus: Viral antigens may be derived from a Togavirus, such as a Rubivirus, an Alphavirus, or an Arterivirus. Antigens derived from Rubivirus, such as Rubella virus, are preferred. Togavirus antigens may be selected from E1, E2, E3, C, NSP-1, NSPO-2, NSP-3 or NSP-4. Togavirus antigens are preferably selected from E1, E2 or E3. Commercially available Rubella vaccines include a live cold-adapted virus, typically in combination with mumps and measles vaccines (MMR).

Flavivirus: Viral antigens may be derived from a Flavivirus, such as Tick-borne encephalitis (TBE), Dengue (types 1, 2, 3 or 4), Yellow Fever, Japanese encephalitis, West Nile encephalitis, St. Louis encephalitis, Russian spring-summer encephalitis, Powassan encephalitis. Flavivirus antigens may be selected from PrM, M, C, E, NS-1, NS-2a, NS2b, NS3, NS4a, NS4b, and NS5. Flavivirus antigens are preferably selected from PrM, M and E. Commercially available TBE vaccine include inactivated virus vaccines.

Pestivirus: Viral antigens may be derived from a Pestivirus, such as Bovine viral diarrhea (BVDV), Classical swine fever (CSFV) or Border disease (BDV).

Hepadnavirus: Viral antigens may be derived from a Hepadnavirus, such as Hepatitis B virus. Hepadnavirus antigens may be selected from surface antigens (L, M and S), core antigens (HBc, HBe). Commercially available HBV vaccines include subunit vaccines comprising the surface antigen S protein.

Hepatitis C virus: Viral antigens may be derived from a Hepatitis C virus (HCV). HCV antigens may be selected from one or more of E1, E2, E1/E2, NS345 polyprotein, NS 345-core polyprotein, core, and/or peptides from the nonstructural regions (Houghton et al., Hepatology (1991) 14:381).

Rhabdovirus: Viral antigens may be derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS). Commercially available Rabies virus vaccine comprise killed virus grown on human diploid cells or fetal rhesus lung cells.

Caliciviridae; Viral antigens may be derived from Calciviridae, such as Norwalk virus, and Norwalk-like Viruses, such as Hawaii Virus and Snow Mountain Virus.

Coronavirus: Viral antigens may be derived from a Coronavirus, SARS, Human respiratory Coronavirus, Avian infectious bronchitis (IBV), Mouse hepatitis virus (MHV), and Porcine transmissible gastroenteritis virus (TGEV). Coronavirus antigens may be selected from spike (S), envelope (E), matrix (M), nucleocapsid (N), and Hemagglutinin-esterase glycoprotein (HE). Preferably, the Coronavirus antigen is derived from a SARS virus. SARS viral antigens are described in WO 04/92360;

Retrovirus: Viral antigens may be derived from a Retrovirus, such as an Oncovirus, a Lentivirus or a Spumavirus. Oncovirus antigens may be derived from HTLV-1, HTLV-2 or HTLV-5. Lentivirus antigens may be derived from HIV-1 or HIV-2. Retrovirus antigens may be selected from gag, pol, env, tax, tat, rex, rev, nef, vif, vpu, and vpr. HIV antigens may be selected from gag (p24gag and p55gag), env (gp160 and gp41), pol, tat, nef, rev vpu, miniproteins, (preferably p55 gag and gp140v delete). HIV antigens may be derived from one or more of the following strains: HIVIIIb, HIVSF2, HIVLAV, HIVLAI, HIVMN, HIV-1CM235, HIV-1US4.

Reovirus: Viral antigens may be derived from a Reovirus, such as an Orthoreovirus, a Rotavirus, an Orbivirus, or a Coltivirus. Reovirus antigens may be selected from structural proteins λ1, λ2, λ3, μ1, μ2, σ1, σ2, or σ3, or nonstructural proteins σNS, μNS, or σ1s. Preferred Reovirus antigens may be derived from a Rotavirus. Rotavirus antigens may be selected from VP1, VP2, VP3, VP4 (or the cleaved product VP5 and VP8), NSP 1, VP6, NSP3, NSP2, VP7, NSP4, or NSP5. Preferred Rotavirus antigens include VP4 (or the cleaved product VP5 and VP8), and VP7.

Parvovirus: Viral antigens may be derived from a Parvovirus, such as Parvovirus B19. Parvovirus antigens may be selected from VP-1, VP-2, VP-3, NS-1 and NS-2. Preferably, the Parvovirus antigen is capsid protein VP-2.

Delta hepatitis virus (HDV): Viral antigens may be derived HDV, particularly δ-antigen from HDV (see, e.g., U.S. Pat. No. 5,378,814).

Hepatitis E virus (HEV): Viral antigens may be derived from HEV.

Hepatitis G virus (HGV): Viral antigens may be derived from HGV.

Human Herpesvirus: Viral antigens may be derived from a Human Herpesvirus, such as Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). Human Herpesvirus antigens may be selected from immediate early proteins (α), early proteins (β), and late proteins (γ). HSV antigens may be derived from HSV-1 or HSV-2 strains. HSV antigens may be selected from glycoproteins gB, gC, gD and gH, fusion protein (gB), or immune escape proteins (gC, gE, or gI). VZV antigens may be selected from core, nucleocapsid, tegument, or envelope proteins. A live attenuated VZV vaccine is commercially available. EBV antigens may be selected from early antigen (EA) proteins, viral capsid antigen (VCA), and glycoproteins of the membrane antigen (MA). CMV antigens may be selected from capsid proteins, envelope glycoproteins (such as gB and gH), and tegument proteins

Papovaviruses: Antigens may be derived from Papovaviruses, such as Papillomaviruses and Polyomaviruses. Papillomaviruses include HPV serotypes 1, 2, 4, 5, 6, 8, 11, 13, 16, 18, 31, 33, 35, 39, 41, 42, 47, 51, 57, 58, 63 and 65. Preferably, HPV antigens are derived from serotypes 6, 11, 16 or 18. HPV antigens may be selected from capsid proteins (L1) and (L2), or E1-E7, or fusions thereof. HPV antigens are preferably formulated into virus-like particles (VLPs). Polyomyavirus viruses include BK virus and JK virus. Polyomavirus antigens may be selected from VP1, VP2 or VP3.

Further provided are antigens, compositions, methods, and microbes included in Vaccines, 4th Edition (Plotkin and Orenstein ed. 2004); Medical Microbiology 4th Edition (Murray et al. ed. 2002); Virology, 3rd Edition (W. K. Joklik ed. 1988); Fundamental Virology, 2nd Edition (B. N. Fields and D. M. Knipe, eds. 1991), which are contemplated in conjunction with the compositions of the present invention.

C. Fungal Antigens

Fungal antigens for use in the invention may be derived from one or more of the fungi set forth below.

Fungal antigens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens may be derived from Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing fungal antigens are well known in the art (see U.S. Pat. No. 6,333,164). In a preferred method a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

D. STD Antigens

The compositions of the invention may include one or more antigens derived from a sexually transmitted disease (STD). Such antigens may provide for prophylactic or therapy for STD's such as chlamydia, genital herpes, hepatitis (such as HCV), genital warts, gonorrhoea, syphilis and/or chancroid (See, WO00/15255). Antigens may be derived from one or more viral or bacterial STD's. Viral STD antigens for use in the invention may be derived from, for example, HIV, herpes simplex virus (HSV-1 and HSV-2), human papillomavirus (HPV), and hepatitis (HCV). Bacterial STD antigens for use in the invention may be derived from, for example, Neiserria gonorrhoeae, Chlamydia trachomatis, Treponema pallidum, Haemophilus ducreyi, E. coli, and Streptococcus agalactiae. Examples of specific antigens derived from these pathogens are described above.

E. Respiratory Antigens

The compositions of the invention may include one or more antigens derived from a pathogen which causes respiratory disease. For example, respiratory antigens may be derived from a respiratory virus such as Orthomyxoviruses (influenza), Pneumovirus (RSV), Paramyxovirus (PIV), Morbillivirus (measles), Togavirus (Rubella), VZV, and Coronavirus (SARS). Respiratory antigens may be derived from a bacteria which causes respiratory disease, such as Streptococcus pneumoniae, Pseudomonas aeruginosa, Bordetella pertussis, Mycobacterium tuberculosis, Mycoplasma pneumoniae, Chlamydia pneumoniae, Bacillus anthracis, and Moraxella catarrhalis. Examples of specific antigens derived from these pathogens are described above.

F. Pediatric Vaccine Antigens

The compositions of the invention may include one or more antigens suitable for use in pediatric subjects. Pediatric subjects are typically less than about 3 years old, or less than about 2 years old, or less than about 1 years old. Pediatric antigens may be administered multiple times over the course of 6 months, 1, 2 or 3 years. Pediatric antigens may be derived from a virus which may target pediatric populations and/or a virus from which pediatric populations are susceptible to infection. Pediatric viral antigens include antigens derived from one or more of Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), and Varicella-zoster virus (VZV), Epstein Barr virus (EBV). Pediatric bacterial antigens include antigens derived from one or more of Streptococcus pneumoniae, Neisseria meningitides, Streptococcus pyogenes (Group A Streptococcus), Moraxella catarrhalis, Bordetella pertussis, Staphylococcus aureus, Clostridium tetani (Tetanus), Cornynebacterium diphtheriae (Diphtheria), Haemophilus influenzae B (Hib), Pseudomonas aeruginosa, Streptococcus agalactiae (Group B Streptococcus), and E. coli. Examples of specific antigens derived from these pathogens are described above.

G. Antigens Suitable for Use in Elderly or Immunocompromised Individuals

The compositions of the invention may include one or more antigens suitable for use in elderly or immunocompromised individuals. Such individuals may need to be vaccinated more frequently, with higher doses or with adjuvanted formulations to improve their immune response to the targeted antigens. Antigens which may be targeted for use in Elderly or Immunocompromised individuals include antigens derived from one or more of the following pathogens: Neisseria meningitides, Streptococcus pneumoniae, Streptococcus pyogenes (Group A Streptococcus), Moraxella catarrhalis, Bordetella pertussis, Staphylococcus aureus, Staphylococcus epidermis, Clostridium tetani (Tetanus), Cornynebacterium diphtheriae (Diphtheria), Haemophilus influenzae B (Hib), Pseudomonas aeruginosa, Legionella pneumophila, Streptococcus agalactiae (Group B Streptococcus), Enterococcus faecalis, Helicobacter pylori, Clamydia pneumoniae, Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), Varicella-zoster virus (VZV), Epstein Barr virus (EBV), Cytomegalovirus (CMV). Examples of specific antigens derived from these pathogens are described above.

H. Antigens Suitable for Use in Adolescent Vaccines

The compositions of the invention may include one or more antigens suitable for use in adolescent subjects. Adolescents may be in need of a boost of a previously administered pediatric antigen. Pediatric antigens which may be suitable for use in adolescents are described above. In addition, adolescents may be targeted to receive antigens derived from an STD pathogen in order to ensure protective or therapeutic immunity before the beginning of sexual activity. STD antigens which may be suitable for use in adolescents are described above.

I. Antigen Formulations

In other aspects of the invention, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(α-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In certain embodiments, the biodegradable polymer is present at a concentration of about 3% to about 10% relative to the organic solvent.

Microparticles for use herein will be formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. Preferably, microparticles for use with the present invention are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Further antigens may also include an outer membrane vesicle (OMV) preparation.

Additional formulation methods and antigens (especially tumor antigens) are provided in U.S. patent Ser. No. 09/581,772.

J. Antigen References

The following references include antigens useful in conjunction with the compositions of the present invention:
1 International patent application WO99/24578
2 International patent application WO99/36544.
3 International patent application WO99/57280.
4 International patent application WO00/22430.
5 Tettelin et al. (2000) Science 287:1809-1815.
6 International patent application WO96/29412.
7 Pizza et al. (2000) Science 287:1816-1820.
8 PCT WO 01/52885.
9 Bjune et al. (1991) Lancet 338(8775).
10 Fuskasawa et al. (1999) Vaccine 17:2951-2958.
11 Rosenqist et al. (1998) Dev. Biol. Strand 92:323-333.
12 Constantino et al. (1992) Vaccine 10:691-698.
13 Constantino et al. (1999) Vaccine 17:1251-1263.
14 Watson (2000) Pediatr Infect Dis J 19:331-332.
15 Rubin (20000) Pediatr Clin North Am 47:269-285,v.
16 Jedrzejas (2001) Microbiol Mol Biol Rev 65:187-207.
17 International patent application filed on 3 Jul. 2001 claiming priority from GB-0016363.4; WO 02/02606; PCT IB/01/00166.
18 Kalman et al. (1999) Nature Genetics 21:385-389.
19 Read et al. (2000) Nucleic Acids Res 28:1397-406.
20 Shirai et al. (2000) J. Infect. Dis 181(Suppl 3):S524-S527.
21 International patent application WO99/27105.
22 International patent application WO00/27994.
23 International patent application WO00/37494.
24 International patent application WO99/28475.
25 Bell (2000) Pediatr Infect Dis J 19:1187-1188.
26 Iwarson (1995) APMIS 103:321-326.
27 Gerlich et al. (1990) Vaccine 8 Suppl:S63-68 & 79-80.
28 Hsu et al. (1999) Clin Liver Dis 3:901-915.
29 Gastofsson et al. (1996) N. Engl. J. Med. 334-:349-355.
30 Rappuoli et al. (1991) TIBTECH 9:232-238.
31 Vaccines (1988) eds. Plotkin & Mortimer. ISBN 0-7216-1946-0.
32 Del Guidice et al. (1998) Molecular Aspects of Medicine 19:1-70.
33 International patent application WO93/018150.
34 International patent application WO99/53310.
35 International patent application WO98/04702.
36 Ross et al. (2001) Vaccine 19:135-142.
37 Sutter et al. (2000) Pediatr Clin North Am 47:287-308.
38 Zimmerman & Spann (1999) Am Fan Physician 59:113-118, 125-126.
39 Dreensen (1997) Vaccine 15 Suppl”52-6.
40 MMWR Morb Mortal Wkly rep 1998 Jan. 16:47(1):12, 9.
41 McMichael (2000) Vaccine 19 Suppl 1:S101-107.
42 Schuchat (1999) Lancer 353(9146):51-6.
43 GB patent applications 0026333.5, 0028727.6 & 0105640.7.
44 Dale (1999) Infect Disclin North Am 13:227-43, viii.
45 Ferretti et al. (2001) PNAS USA 98: 4658-4663.
46 Kuroda et al. (2001) Lancet 357(9264):1225-1240; see also pages 1218-1219.
47 Ramsay et al. (2001) Lancet 357(9251):195-196.
48 Lindberg (1999) Vaccine 17 Suppl 2:S28-36.
49 Buttery & Moxon (2000) J R Coil Physicians Long 34:163-168.
50 Ahmad & Chapnick (1999) Infect Dis Clin North Am 13:113-133, vii.
51 Goldblatt (1998) J. Med. Microbiol. 47:663-567.
52 European patent 0 477 508.
53 U.S. Pat. No. 5,306,492.
54 International patent application WO98/42721.
55 Conjugate Vaccines (eds. Cruse et al.) ISBN 3805549326, particularly vol. 10:48-114.
56 Hermanson (1996) Bioconjugate Techniques ISBN: 012323368 & 012342335X.
57 European patent application 0372501.
58 European patent application 0378881.
59 European patent application 0427347.
60 International patent application WO93/17712.
61 International patent application WO98/58668.
62 European patent application 0471177.
63 International patent application WO00/56360.
64 International patent application WO00/67161.

The contents of all of the above cited patents, patent applications and journal articles are incorporated by reference as if set forth fully herein.

Where a saccharide or carbohydrate antigen is used, it is preferably conjugated to a carrier protein in order to enhance immunogenicity. See Ramsay et al. (2001) Lancet 357(9251):195-196; Lindberg (1999) Vaccine 17 Suppl 2:S28-36; Buttery & Moxon (2000) J R Coll Physicians Lond 34:163-168; Ahmad & Chapnick (1999) Infect Dis Clin North Am 13:113-133, vii; Goldblatt (1998) J. Med. Microbiol. 47:563-567; European patent 0 477 508; U.S. Pat. No. 5,306,492; WO98/42721; Conjugate Vaccines (eds. Cruse et al.) ISBN 3805549326, particularly vol. 10:48-114; Hermanson (1996) Bioconjugate Techniques ISBN: 0123423368 or 012342335X. Preferred carrier proteins are bacterial toxins or toxoids, such as diphtheria or tetanus toxoids. The CRM197 diphtheria toxoid is particularly preferred.

Other carrier polypeptides include the N. meningitidis outer membrane protein (EP-A-0372501), synthetic peptides (EP-A-0378881 and EP-A 0427347), heat shock proteins (WO 93/17712 and WO 94/03208), pertussis proteins (WO 98/58668 and EP A 0471177), protein D from H. influenzae (WO 00/56360), cytokines (WO 91/01146), lymphokines, hormones, growth factors, toxin A or B from C. difficile (WO 00/61761), iron-uptake proteins (WO 01/72337), etc. Where a mixture comprises capsular saccharide from both serigraphs A and C, it may be preferred that the ratio (w/w) of MenA saccharide:MenC saccharide is greater than 1 (e.g., 2:1, 3:1, 4:1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g., detoxification of pertussis toxin by chemical and/or genetic means.

Pharmaceutically Acceptable Carriers

Compositions of the invention will typically, in addition to the components mentioned above, comprise one or more “pharmaceutically acceptable carriers.” These include any carrier which does not itself induce the production of antibodies harmful to the individual receiving the composition. Suitable carriers typically are large, slowly metabolized macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. A composition may also contain a diluent, such as water, saline, glycerol, etc. Additionally, an auxiliary substance, such as a wetting or emulsifying agent, pH buffering substance, and the like, may be present. A thorough discussion of pharmaceutically acceptable components is available in Gennaro (2000) Remington: The Science and Practice of Pharmacy, 20th ed., ISBN: 0683306472.

### Immunoregulatory Agents

Adjuvants

Vaccines of the invention may be administered in conjunction with other immunoregulatory agents. In particular, compositions will usually include an adjuvant. Adjuvants for use with the invention include, but are not limited to, one or more of the following set forth below:

A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X, Plenum Press), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO00/23105).

Aluminum salts may be included in vaccines of the invention such that the dose of Al³⁺ is between 0.2 and 1.0 mg per dose.

In one embodiment the aluminum based adjuvant for use in the present invention is alum (aluminum potassium sulfate (AlK(SO₄)₂)), or an alum derivative, such as that formed in-situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbent, having a surface area of approximately 500 m²/g. Alternatively, aluminum phosphate adjuvant (AlPO₄) or aluminum hydroxyphosphate, which contains phosphate groups in place of some or all of the hydroxyl groups of aluminum hydroxide adjuvant is provided. Preferred aluminum phosphate adjuvants provided herein are amorphous and soluble in acidic, basic and neutral media.

In another embodiment the adjuvant of the invention comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. More particular still, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (isoelectric point=4) adsorbs lysozyme, but not albumin at pH 7.4. Should albumin be the target, aluminum hydroxide adjuvant would be selected (iep 11.4). Alternatively, pretreatment of aluminum hydroxide with phosphate lowers its isoelectric point, making it a preferred adjuvant for more basic antigens.

B. Oil-Emulsions

Oil-emulsion compositions suitable for use as adjuvants in the invention include squalene-water emulsions, such as MF59 (5% Squalene, 0.5% TWEEN™ 80, and 0.5% Span 85, formulated into submicron particles using a microfluidizer). See WO90/14837. See also, Podda, Vaccine (2001) 19: 2673-2680; Frey et al., Vaccine (2003) 21:4234-4237. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v TWEEN™ 80 (polyoxyelthylenesorbitan monooleate), and/or 0.25-1.0% SPAN 85™ (sorbitan trioleate), and, optionally, N-acetylmuramyl-L-alanyl-D-isoglutaminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (International Publication No. WO90/14837; U.S. Pat. Nos. 6,299,884 and 6,451,325, and Ott et al., in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v TWEEN™ 80, and 0.5% w/v SPAN 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v TWEEN™ 80, and 0.75% w/v SPAN 85™ and optionally MTP-PE. Yet another submicron oil-in-water emulsion is MF75, also known as SAF, containing 10% squalene, 0.4% TWEEN™ 80, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in WO90/14837 and U.S. Pat. Nos. 6,299,884 and 6,451,325.

Complete Freund's adjuvant (CFA) and incomplete Freund's adjuvant (IFA) may also be used as adjuvants in the invention.

C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of (an) additional detergent(s). See WO00/07621.

A review of the development of saponin based adjuvants can be found in Barr, et al., Advanced Drug Delivery Reviews (1998) 32:247-271. See also Sjolander, et al., Advanced Drug Delivery Reviews (1998) 32:321-338.

D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., Virology (2002) 293:273-280; Lenz et al., Journal of Immunology (2001) 5246-5355; Pinto, et al., Journal of Infectious Diseases (2003) 188:327-338; and Gerber et al., Journal of Virology (2001) 75(10):4752-4760. Virosomes are discussed further in, for example, Gluck et al., Vaccine (2002) 20:B10-B16. Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

E. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC 529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., Vaccine (2003) 21:2485-2491; and Pajak, et al., Vaccine (2003) 21:836-842.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpGs can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., Nucleic Acids Research (2003) 31(9): 2393-2400; WO02/26757 and WO99/62923 for examples of possible analog substitutions. The adjuvant effect of CpG oligonucleotides is further discussed in Krieg, Nature Medicine (2003) 9(7): 831-835; McCluskie, et al., FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., J. Immunol. (2003) 170(8):4061-4068; Krieg, TRENDS in Immunology (2002) 23(2): 64-65 and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., BBRC (2003) 306:948-953; Kandimalla, et al., Biochemical Society Transactions (2003) 31(part 3):664-658; Bhagat et al., BBRC (2003) 300:853-861 and WO03/035836.

(4) ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon, et al., Infection and Immunity (2002) 70(6):3012-3019; Pizza, et al., Vaccine (2001) 19:2534-2541; Pizza, et al., Int. J. Med. Microbiol. (2000) 290(4-5):455-461; Scharton-Kersten et al., Infection and Immunity (2000) 68(9):5306-5313; Ryan et al., Infection and Immunity (1999) 67(12):6270-6280; Partidos et al., Immunol. Lett. (1999) 67(3):209-216; Peppoloni et al., Vaccines (2003) 2(2):285-293; and Pine et al., (2002) J. Control Release (2002) 85(1-3):263-270. Numerical reference for amino acid substitutions is preferably based on the alignments of the A and B subunits of ADP-ribosylating toxins set forth in Domenighini et al., Mol. Microbiol. (1995) 15(6):1165-1167.

F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention. See WO99/27960.

G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non toxic (e.g. a poly(α-hydroxy acid), a polyhydroxybutyric acid, a polyorthoester, a polyanhydride, a polycaprolactone, etc.), with poly(lactide co glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

K. Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N acetylmuramyl-1-alanyl-d-isoglutaminyl-1-alanine-2-(β-2′-dipalmitoyl-sn-glycero-3-hydroxyphosphoryloxy)-ethylamine MTP-PE).

L. Imidazoquinoline Compounds.

Examples of imidazoquinoline compounds suitable for use adjuvants in the invention include Imiquimod and its analogues, described further in Stanley, Clin Exp Dermatol (2002) 27(7):571-577; Jones, Curr Opin Investig Drugs (2003) 4(2):214-218; and U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612.

M. Thiosemicarbazone Compounds.

Examples of thiosemicarbazone compounds, as well as methods of formulating, manufacturing, and screening for compounds all suitable for use as adjuvants in the invention include those described in WO04/60308. The thiosemicarbazones are particularly effective in the stimulation of human peripheral blood mononuclear cells for the production of cytokines, such as TNF-α.

N. Tryptanthrin Compounds.

Examples of tryptanthrin compounds, as well as methods of formulating, manufacturing, and screening for compounds all suitable for use as adjuvants in the invention include those described in WO04/64759. The tryptanthrin compounds are particularly effective in the stimulation of human peripheral blood mononuclear cells for the production of cytokines, such as TNF-α.

The invention may also comprise combinations of aspects of one or more of the adjuvants identified above. For example, the following adjuvant compositions may be used in the invention:
(1) a saponin and an oil-in-water emulsion (WO99/11241);
(2) a saponin (e.g., QS21)+a non-toxic LPS derivative (e.g. 3dMPL) (see WO94/00153);
(3) a saponin (e.g., QS21)+a non-toxic LPS derivative (e.g. 3dMPL)+a cholesterol;
(4) a saponin (e.g., QS21)+3dMPL+IL 12 (optionally+a sterol) (WO98/57659);
(5) combinations of 3dMPL with, for example, QS21 and/or oil-in-water emulsions (See European patent applications 0835318, 0735898 and 0761231);
(6) SAF, containing 10% Squalane, 0.4% Tween 80, 5% pluronic-block polymer L121, and thr-MDP, either microfluidized into a submicron emulsion or vortexed to generate a larger particle size emulsion.
(7) RIBI™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DETOX™); and
(8) one or more mineral salts (such as an aluminum salt)+a non-toxic derivative of LPS (such as 3dPML).
(9) one or more mineral salts (such as an aluminum salt)+an immunostimulatory oligonucleotide (such as a nucleotide sequence including a CpG motif).

O. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

Aluminum salts and MF59 are preferred adjuvants for use with injectable influenza vaccines. Bacterial toxins and bioadhesives are preferred adjuvants for use with mucosally-delivered vaccines, such as nasal vaccines.

The contents of all of the above cited patents, patent applications and journal articles are incorporated by reference as if set forth fully herein.

### Therapeutic Methods

The invention provides methods for inducing or increasing an immune response to S. pyogenes using the compositions described above. The immune response is preferably protective and can include antibodies and/or cell-mediated immunity (including systemic and mucosal immunity). Immune responses include booster responses.

The combinations of GAS antigens, nucleic acid molecules or antibodies described above may be included in a single composition for simultaneous administration. Alternatively, the combinations of GAS antigens, nucleic acid molecules or antibodies may be administered sequentially. For example, where the combination comprises Spy0167, Spy0269, and Spy0416 or mutants or fragments thereof, these 3 antigens may be administered simultaneously in a single composition or sequentially in separate compositions. In this situation, the invention provides: Spy0167 for administration to an animal that has already received Spy0269 and/or Spy416; Spy0269 for administration to an animal that has already received Spy0167 and/or Spy0416; and Spy0416 for administration to an animal that has already received Spy0167 and/or Spy0269.

Teenagers and children, including toddles and infants, can receive a vaccine for prophylactic use; therapeutic vaccines typically are administered to teenagers or adults. A vaccine intended for children may also be administered to adults e.g., to assess safety, dosage, immunogenicity, etc.

Diseases caused by Streptococcus pyogenes which compositions of the invention can reduce the risk of, prevent, or treat include, but are not limited to, pharyngitis (such as streptococcal sore throat), scarlet fever, impetigo, erysipelas, cellulitis, septicemia, toxic shock syndrome, necrotizing fasciitis, and sequelae such as rheumatic fever and acute glomerulonephritis. The compositions may also be effective against other streptococcal bacteria, e.g., GBS.

Tests to Determine the Efficacy of the Immune Response

One way of assessing efficacy of therapeutic treatment involves monitoring GAS infection after administration of the composition of the invention. One way of assessing efficacy of prophylactic treatment involves monitoring immune responses against the GAS antigens in the compositions of the invention after administration of the composition.

Another way of assessing the immunogenicity of the component proteins of the immunogenic compositions of the present invention is to express the GAS antigens recombinantly and to screen patient sera or mucosal secretions by immunoblot. A positive reaction between the protein and the patient serum indicates that the patient has previously mounted an immune response to the protein in question; i.e., the protein is an immunogen. This method may also be used to identify immunodominant proteins and/or epitopes.

Another way of checking efficacy of therapeutic treatment involves monitoring GAS infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against GAS challenge after administration of the composition. Typically, serum specific antibody responses are determined post-immunization but pre-challenge whereas mucosal specific antibody body responses are determined post-immunization and post-challenge.

The vaccine compositions of the present invention can be evaluated in in vitro and in vivo animal models prior to host, e.g., human, administration. Particularly useful mouse models include those in which intraperitoneal immunization is followed by either intraperitoneal challenge or intranasal challenge.

The efficacy of immunogenic compositions of the invention can also be determined in vivo by immunizing animal models, (e.g., guinea pigs or mice) with the immunogenic compositions and ascertaining the level of protection obtained after challenge with GAS.

In vivo efficacy models include but are not limited to: (i) a murine infection model using human GAS serotypes; (ii) a murine disease model which is a murine model using a mouse-adapted GAS strain, such as the M23 strain which is particularly virulent in mice, and (iii) a primate model using human GAS isolates.

The immune response may be one or both of a TH1 immune response and a TH2 response. The immune response may be an improved or an enhanced or an altered immune response. The immune response may be one or both of a systemic and a mucosal immune response. Preferably the immune response is an enhanced system and/or mucosal response.

An enhanced systemic and/or mucosal immunity is reflected in an enhanced TH1 and/or TH2 immune response. Preferably, the enhanced immune response includes an increase in the production of IgG1 and/or IgG2a and/or IgA.

Preferably the mucosal immune response is a TH2 immune response. Preferably, the mucosal immune response includes an increase in the production of IgA.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH1 immune response may include one or more of an increase in CTLs, an increase in one or more of the cytokines associated with a TH1 immune response (such as IL-2, IFNγ, and TNFβ), an increase in activated macrophages, an increase in NK activity, or an increase in the production of IgG2a. Preferably, the enhanced TH1 immune response will include an increase in IgG2a production.

Immunogenic compositions of the invention may be used either alone or in combination with other GAS antigens optionally with an immunoregulatory agent capable of eliciting a Th1 and/or Th2 response.

The invention also comprises an immunogenic composition comprising one or more immunoregulatory agent, such as a mineral salt, such as an aluminium salt and an oligonucleotide containing a CpG motif. Most preferably, the immunogenic composition includes both an aluminium salt and an oligonucleotide containing a CpG motif. Alternatively, the immunogenic composition includes an ADP ribosylating toxin, such as a detoxified ADP ribosylating toxin and an oligonucleotide containing a CpG motif. Preferably, one or more of the immunoregulatory agents include an adjuvant. The adjuvant may be selected from one or more of the group consisting of a TH1 adjuvant and TH2 adjuvant.

The compositions of the invention will preferably elicit both a cell mediated immune response as well as a humoral immune response in order to effectively address a GAS infection. This immune response will preferably induce long lasting (e.g., neutralizing) antibodies and a cell mediated immunity that can quickly respond upon exposure to one or more GAS antigens.

In one particularly preferred embodiment, the immunogenic composition comprises one or more GAS antigens which elicit(s) a neutralizing antibody response and one or more GAS antigens which elicit(s) a cell mediated immune response. In this way, the neutralizing antibody response prevents or inhibits an initial GAS infection while the cell-mediated immune response capable of eliciting an enhanced Th1 cellular response prevents further spreading of the GAS infection.

Compositions of the invention will generally be administered directly to a patient. The compositions of the present invention may be administered, either alone or as part of a composition, via a variety of different routes. Certain routes may be favored for certain compositions, as resulting in the generation of a more effective immune response, preferably a CMI response, or as being less likely to induce side effects, or as being easier for administration.

Delivery methods include parenteral injection (e.g., subcutaneous, intraperitoneal, intravenous, intramuscular, or interstitial injection) and rectal, oral (e.g., tablet, spray), vaginal, topical, transdermal (e.g., see WO 99/27961), transcutaneous (e.g., see WO02/074244 and WO02/064162), intranasal (e.g., see WO03/028760), ocular, aural, and pulmonary or other mucosal administration.

By way of example, the compositions of the present invention may be administered via a systemic route or a mucosal route or a transdermal route or it may be administered directly into a specific tissue. As used herein, the term “systemic administration” includes but is not limited to any parenteral routes of administration. In particular, parenteral administration includes but is not limited to subcutaneous, intraperitoneal, intravenous, intraarterial, intramuscular, or intrasternal injection, intravenous, intraarterial, or kidney dialytic infusion techniques. Preferably, the systemic, parenteral administration is intramuscular injection. As used herein, the term “mucosal administration” includes but is not limited to oral, intranasal, intravaginal, intrarectal, intratracheal, intestinal and ophthalmic administration.

Dosage treatment can be a single dose schedule or a multiple dose schedule. Multiple doses may be used in a primary immunization schedule and/or in a booster immunization schedule. In a multiple dose schedule the various doses may be given by the same or different routes e.g., a parenteral prime and mucosal boost, a mucosal prime and parenteral boost, etc.

The compositions of the invention may be prepared in various forms. For example, a composition can be prepared as an injectable, either as a liquid solution or a suspension. Solid forms suitable for solution in, or suspension in, liquid vehicles prior to injection can also be prepared (e.g., a lyophilized composition). A composition can be prepared for oral administration, such as a tablet or capsule, as a spray, or as a syrup (optionally flavored). A composition can be prepared for pulmonary administration, e.g., as an inhaler, using a fine powder or a spray. A composition can be prepared as a suppository or pessary. A composition can be prepared for nasal, aural or ocular administration e.g., as drops. A composition can be in kit form, designed such that a combined composition is reconstituted just prior to administration to a patient. Such kits may comprise one or more mutant Spy0167 or other antigens in liquid form and one or more lyophilized antigens.

Immunogenic compositions used as vaccines comprise an immunologically effective amount of the GAS antigens or other antigens, as well as any other components, as needed, such as antibiotics. An “immunologically effective amount” is an amount which, when administered to an individual, either in a single dose or as part of a series, increases a measurable immune response or prevents or reduces a clinical symptom.

The immunogenic compositions of the present invention may be administered in combination with an antibiotic treatment regime. In one embodiment, the antibiotic is administered prior to administration of a composition of the invention. In another embodiment, the antibiotic is administered subsequent to the administration of a composition of the invention. Examples of antibiotics suitable for use in the treatment of a GAS infection include but are not limited to penicillin or a derivative thereof or clindamycin, cephalosporins, glycopeptides (e.g., vancomycin), and cycloserine.

The amount of active agents in a composition varies depending upon the health and physical condition of the individual to be treated, age, the taxonomic group of individual to be treated (e.g., non-human primate, primate, etc.), the capacity of the individual's immune system to synthesize antibodies, the degree of protection desired, the formulation of the vaccine, the treating doctor's assessment of the medical situation, and other relevant factors. The amount will fall in a relatively broad range which can be determined through routine trials.

### Kits

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other buffers, diluents, filters, needles, and syringes. The kit can also comprise a second or third container with another active agent, for example an antibiotic.

The kit can also comprise a package insert containing written instructions for methods of inducing immunity against S. pyogenes or for treating S. pyogenes infections. The package insert can be an unapproved draft package insert or can be a package insert approved by the Food and Drug Administration (FDA) or other regulatory body.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### Example 1

### Hemolysis Assays

Serial dilutions of Spy0167 or a Spy0167 mutant are prepared in 96-well plates with U-shaped bottoms using PBS+0.5% BSA. One ml of sheep blood is washed three times in PBS (with centrifugation at 3000×g), and blood cells are suspended in 5 ml of PBS. An equal volume of suspension is added to 50 μl of each toxin dilution and incubated at 37° C. for 30 min. Triton (2%) in water is used to give 100% hemolysis, and PBS+0.5% BSA is used as negative control. Plates are then centrifuged for 5 min at 1,000×g, and the supernatant is transferred carefully to 96-well flat-bottomed plates. The absorbance is read at 540 nm. One hemolytic unit (HU) is defined as the amount of Spy0167 or Spy0167 mutant required to obtained 50% of maximum lysis obtained treating the blood cells with 2% Triton.

### Example 2

### Assessment of In Vivo Toxicity of Spy0167 Mutant Antigens

Intravenous Injection of Antigen.

A solution of either wild-type or mutant Spy0167 antigen in PBS is diluted in a solution of PBS+2 mM DTT, then 100 ml is injected into the tail vein of a mouse. Mice are observed for 2-3 days. Injection of wild-type Spy0167 typically results in death within a few minutes.

In Vivo Lethality Inhibition Assay.

For lethality inhibition mediated by immune sera, 10 μg/mouse of wild-type Spy0167 (a solution of 100 μg/ml in PBS, 2 mM DTT) are incubated for 20 minutes with rotation “end over end” at room temperature with either anti-Spy0167 serum or control serum (obtained from mice immunized with adjuvant alone). After incubation, the samples are inoculated in the mice by intravenous injection into the tail vein. Mice are observed for 2-3 days.

Acute In Vivo Toxicity.

Acute in vivo toxicity is assessed using a dose of 10 μg/mouse of wild-type Spy0167 as a positive control and injection of Freund's adjuvant alone as a negative control. Ten μg/mouse of wild-type Spy0167 are incubated with either wild-type Spy0167 antiserum or with control serum and inoculated into mice as described above.

### Example 3

### Inactivation of Spy0416 Proteolytic Activity

SDS-PAGE.

IL-8 is incubated with wild-type Spy0416 or a Spy0416 mutant. The incubation mixtures is loaded on SDS-PAGE and revealed by silver staining Wild-type Spy0416 releases two bands: 8 kDa (active form) and 6 kDa (inactive cleaved IL-8). A Spy0416 mutant releases only one band, which corresponds to uncleaved IL-8, as in the control reaction (without enzyme).

ELISA.

IL-8 is incubated with wild-type Spy0416 or a Spy0416 mutant at three different concentrations, and the incubation mixtures are tested for the presence of uncleaved IL-8 using an antibody which is specific for the cytokine but which is unable to recognize the cleaved inactive form. The results are expressed as percentage of uncleaved IL-8 after 0, 8 and 24 h reactions, and were calculated as follows:

$$\frac{[ IL \text{-} 8 in the reaction mix ]}{[ IL \text{-} 8 in the control mix ] ,} \times 100$$

where “control mix” is the reaction mix without the enzyme at time point 0.

### Example 4

### The Protective Capacity of GAS Antigens

A GAS antigen is used to immunize mice to test its capacity to confer protection against GAS lethal challenge. The antigen is administered intraperitoneally, optionally with an adjuvant, at days 0, 21, and 35. Blood samples are taken two weeks after the third immunization. The mice are then challenged intranasally with a GAS strain (e.g., 10⁸ cfu of GAS strain 3348 M1 in 50 μl). Survival is monitored for a 10-14 day period.

### Example 5

### Dose-Dependent Inhibition of Spy0416-Mediated IL-8 Cleavage by Spy0416 Antibodies

Antisera specific for Spy0416, wild type and inactive mutants, are produced by immunizing CD1 mice with purified recombinant proteins. IL-8 (10 μg/ml) is incubated with wild-type Spy0416 with or without Spy0416 antiserum (1:50 and 1:5000), or with monoclonal antibodies raised against wild-type Spy0416, in two different conditions: (1) 8 hour incubation, 0.1 μg/ml of Spy0416 and (2) 24 hour incubation, 0.05 μg/ml of Spy0416. The incubation mixtures are then tested for the presence of uncleaved IL-8 by ELISA. The results demonstrate a dose-dependent inhibition of Spy0416-mediated IL-8 cleavage by the Spy0416 antiserum or monoclonal antibodies.

### Example 6

### Inhibition of Spy0167 Hemolysis by Antibodies Against Wild-Type or Mutant Spy0167 (Spy0167)

Using 50 ng/ml (3.5 HU) of toxin, the antibody titer required to obtain 50% reduction of Spy0167 hemolytic activity is tested using an adjuvant (e.g., Freund's adjuvant, Alum, or MF59™). Adjuvant alone is used as a negative control.

### Example 7

### Protective Capacity of the Combination of GAS Antigens in a Subcutaneous Challenge Model

Mice were immunized with single GAS antigens (Spy0167, Spy0416, or Spy0269) or with combinations of GAS antigens GAS (Spy0167+Spy0416+Spy0269; or Spy0416+Spy0269). The mice were then infected subcutaneously with the SF370 M1 strain of GAS, which causes skin lesions. The protective effect of the GAS antigens or antigen combinations was determined by measuring lesion size.

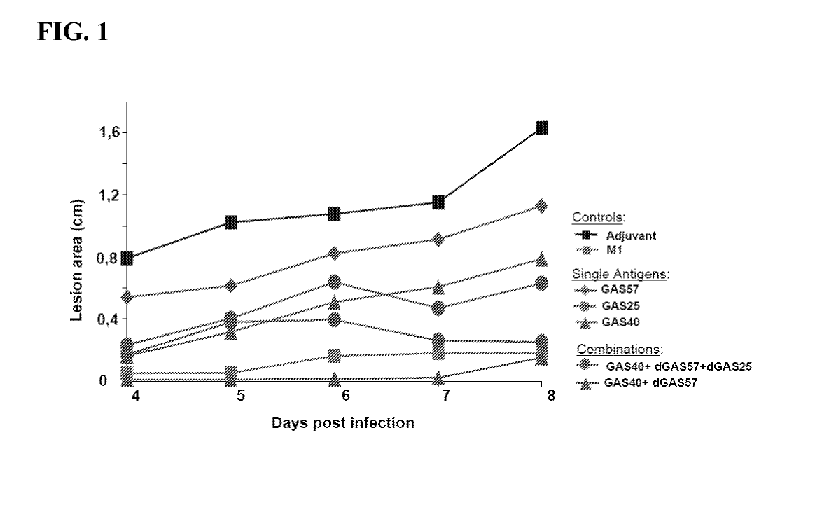

In this model, there is a synergistic protective effect obtained by using the combination of Spy0167+Spy0416+Spy0269 or the combination of Spy0416+Spy0269 compared with the protective effect obtained by using any of these GAS antigens alone. In fact, the protective effect provided by the combinations tested is comparable to that provided using GAS M1 protein. See FIG. 1.

### Example 8

### Protective Capacity of the Combination of Mutant GAS Antigens

The protective capacity of a combination of GAS mutant antigens (Spy0167 mutant antigen P427L/W535F and Spy0416 mutant antigen D151A/S617A) against intranasal challenge with various strains of GAS was tested essentially as described in Example 4. The results are shown in Table 2.

TABLE 2

| | |
|---|---|
| percent survival | no. mice |

| challenge | negative | | | tested/ |
|---|---|---|---|---|
| strain | control | combination | adjuvant | vaccine |
| M1 | 19 | 85 | alum | 128 |
| M2 | 15 | 40 | alum | 32 |
| M6 | 25 | 58 | alum | 80 |
| M12 | 19 | 47 | alum | 144 |
| M23 | 19 | 54 | Freund's | 60 |

### Example 9

### Preparation of Spy0416 Mutants

By comparison with C5a protease, three amino acids in the Spy0416 were identified that putatively constitute the catalytic site of the protease: D151, H279 and S617. In order to obtain an inactive form of the enzyme, nucleotide substitutions resulting in amino acid changes D151A and/or S617A were introduced in the Spy0416 coding sequence by Splicing by Overlapping Extension PCR(SOE-PCR).

Substitution D151A

Three PCR reactions were carried out:

| PCR reaction | Template | Primers |
|---|---|---|
| PCR1 (360 | genomic | 57F, GTGCGT [symbol] GCAGATGAGCT |
| bps) | SF370 | AAGCA; SEQ ID NO: 150 |
| | | 57mutDR1, CCCTGTGGCAATAACTGCG |
| | | AC; SEQ ID NO: 151 |
| PCR2 (910 | genomic | 57mutDF1, cgCAGTTATTGcCACAGGG |
| bp) | SF370 | AT, SEQ ID NO: 152 |
| | | 57mutSalR, |
| | | CTGACTGA [symbol] AGACTCTGAATAGA |
| | | TG, SEQ ID NO: 153 |
| PCR3 (1270 | PCR1, | 57F |
| bps) | PCR2 | 57mutSalR |

PCR product 3 was then digested with Nde-Sal and introduced in pET21_{—}57his digested with the same enzymes. Clones containing the correct in-frame substitutions (pET21_{—}57his_D151A) were selected by DNA sequencing.

Substitution S617A

Three PCR reactions were carried out:

| PCR reaction | Template | Primers |
|---|---|---|
| PCR4 (517 | genomic | 57mutSalF, |
| bp) | SF370 | CTGACTGA [symbol] TTTAAAGACATAAA |
| | | AGATAG; SEQ ID NO: 154 |
| | | 57mutSR1, GAGAGGCCATAGCTGTTCC |
| | | TG; SEQ ID NO: 155 |
| PCR6 (4740 | genomic | 57mutSF1, GGAACAGCTATGGCCTCTC |
| bp) | SF370 | CT; SEQ ID NO: 156 |
| | | 57R |
| PCR6 (5257 | PCR4, | 57FmutSalF |
| bp) | PCR5 | 57R |

PCR product 6 was then digested with Sal-Xho and introduced in pET21_{—}57his digested with the same enzymes. Clones containing the correct in-frame substitutions (pET21_{—}57his_S617A) were selected by DNA sequencing.

Substitution D151A+S617A

PCR product 6 was digested Sal-Xho and introduced in pET21_{—}57his_D151A digested with the same enzymes. Clones containing the correct in-frame substitutions (pET21_{—}57his_D151A+S617A) were selected by DNA sequencing.

The single and double mutant proteins were expressed and purified using three chromatographic steps: ion exchange chromatography (Q Sepharose HP), hydroxylapatite chromatography and gel filtration chromatography.

### Example 10

### Point Mutation D151A Results in Inactivation of Spy0416 Proteolytic Activity

Spy0416 mutant D151A was expressed as a recombinant His-tagged protein. Two types of assays demonstrated that this mutant has lost the ability to cleave IL-8.

SDS-PAGE

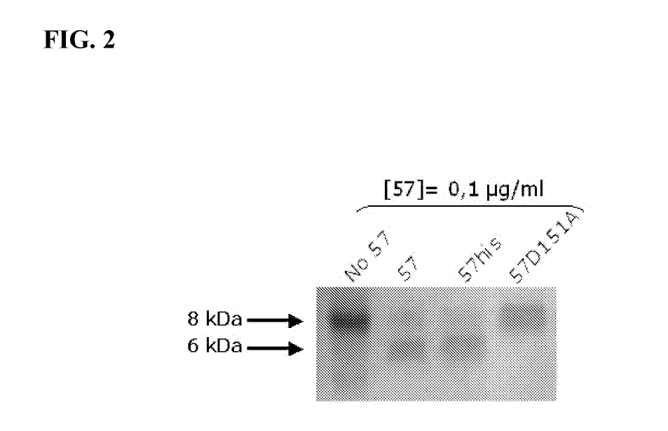

IL-8 was incubated with wild-type Spy0416 or the Spy0416 mutant D151A. The incubation mixtures were loaded on SDS-PAGE and revealed by silver staining. The results are shown in FIG. 2. Wild-type Spy0416 (lanes 2 and 3) released two bands: 8 kDa (active form) and 6 kDa (inactive cleaved IL-8). In contrast, the Spy0416 D151A mutant released only one band, which corresponded to uncleaved IL-8, as in the control reaction (without enzyme).

ELISA

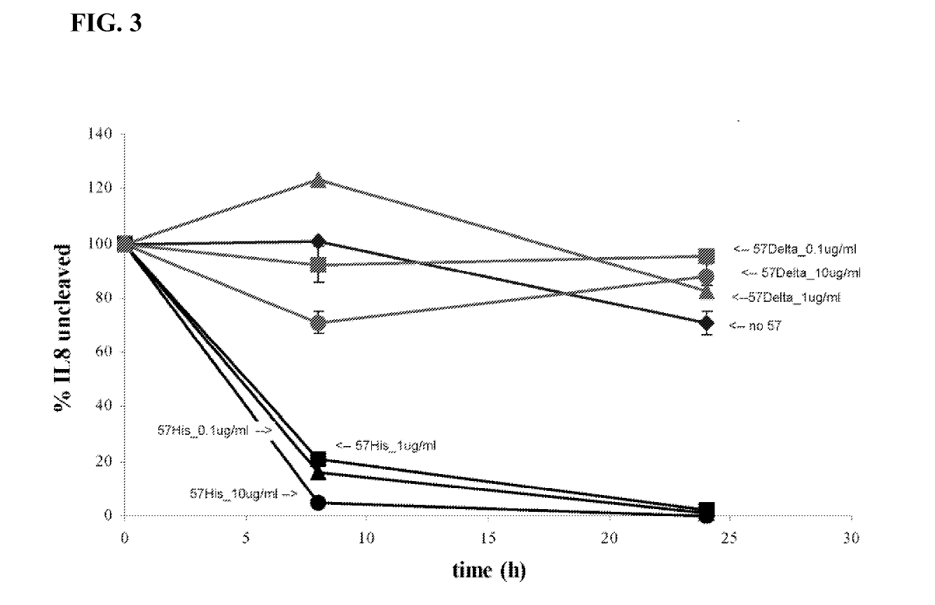

IL-8 was incubated with wild-type Spy0416 or the Spy0416 mutant D151A at three different concentrations, and the incubation mixtures were tested for the presence of uncleaved IL-8 using an antibody which is specific for the cytokine but which is unable to recognize the cleaved inactive form. The results are shown in FIG. 3, expressed as percentage of uncleaved IL-8 after 0, 8 and 24 h reactions, and were calculated as follows:

$$\frac{[ IL \text{-} 8 in the reaction mix ]}{[ IL \text{-} 8 in the control mix ] ,} \times 100$$

where “control mix” is the reaction mix without the enzyme at time point 0.

As shown in FIG. 3, wild-type Spy0416 almost completely inactivated IL-8 after 8 hours, even at the lower concentration, while no inactivation was observed for IL-8 treated with the mutant enzyme.

### Example 11

### Spy0416 Mutant S617A and Spy0416 Double Mutant D151A+S617A do not Cleave IL-8

Spy0416 mutant S617A and Spy0416 double mutant D151A+S617A were expressed as His-tagged proteins and were tested in IL-8 inactivation experiments as described in Example 2.

SDS-PAGE

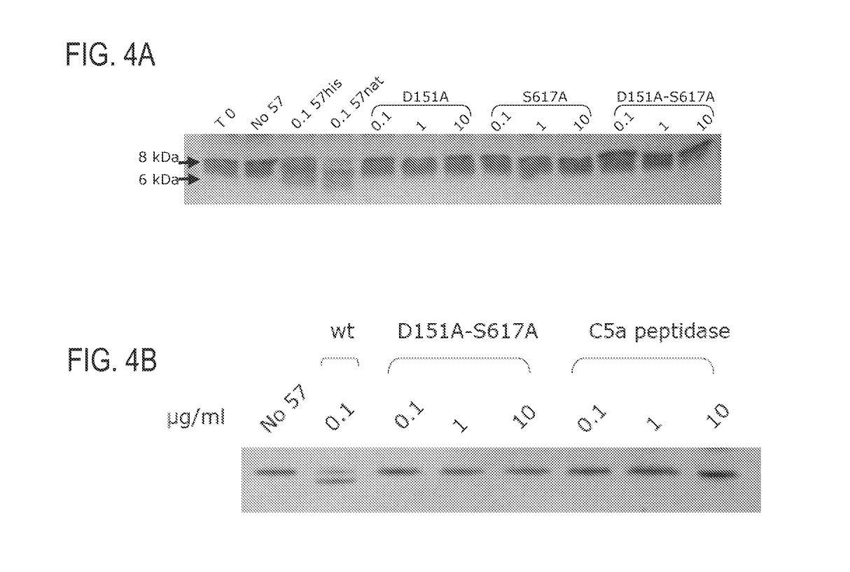

IL-8 was incubated with either wild-type Spy0416 (His-tagged or tag-less), or each of the Spy0416 mutants D151A, S617A and D151AS+S617A for 24 hours. The incubation mixtures were loaded on an SDS-polyacrylamide gel and revealed by silver staining. The results of two experiments are shown in FIGS. 4A and 4B. Both the Spy0416 S617A mutant and the GAS D151+S617A mutant are unable to cleave IL-8, even at a 100-fold higher concentration than wild-type Spy0416.

ELISA

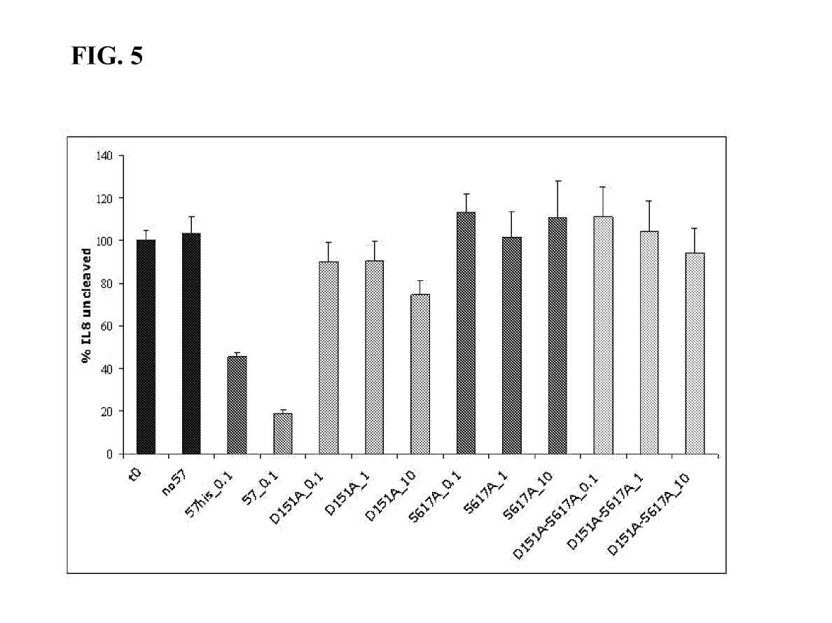

The same samples were used to perform an ELISA assay which confirmed that the single and double amino acid substitutions eliminate the ability of Spy0416 to cleave IL-8. The results, which are shown in FIG. 5, demonstrate that the mutants release 100% of uncleaved IL-8 after 24 h incubation, compared to 20-40% released by wild-type Spy0416.

### Example 12

### The Protective Capacity of Spy0416 Mutants is Similar to that Obtained with Wild-Type Spy0416

The Spy0416 mutants D151A and D151A+S617A were used to immunize mice to test their capacity to confer protection against GAS lethal challenge in comparison to wild-type Spy0416. The results of two experiments (20 mice each) are summarized below and expressed as average % survival.

TABLE 3

| | NO. MICE | NO. DEAD | % SURVIVAL |
|---|---|---|---|
| PBS + Freund | 40 | 26 | 35 |
| 192 M1 + Freund | 20 | 0 | 100 |
| 57 WT + Freund | 40 | 12 | 70 |
| 57 D151A + Freund | 40 | 6 | 85 |
| 57 D151A-S617A + Freund | 40 | 9 | 78 |

### Example 13

### Purified Inactive Mutants Appear as a Single Peptide Compared to Wild-Type Spy0416, which Exists Only in the Form of Two Non Covalently Associated Protein Fragments

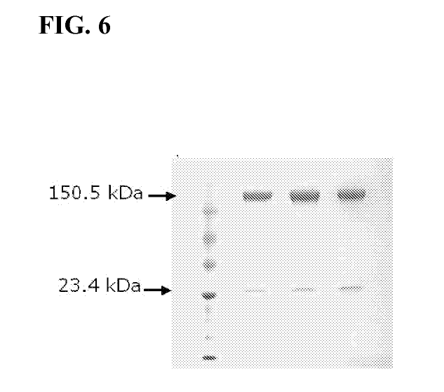

Wild-type Spy0416 is obtained mainly in the form of two fragments, one of about 23 kDa and a one of 150 kDa. The two fragments are not separated in Ni-chelating affinity purification or by gel filtration, but appear as two different bands on SDS-PAGE (FIG. 6). N-terminal sequencing confirmed that the 23 kDa fragment is the N-terminal portion of Spy0416 (amino acids 34-244 of SEQ ID NO:50) while the 150 kDa fragment is the C-terminal region (amino acids 245-1603 of SEQ ID NO:50).

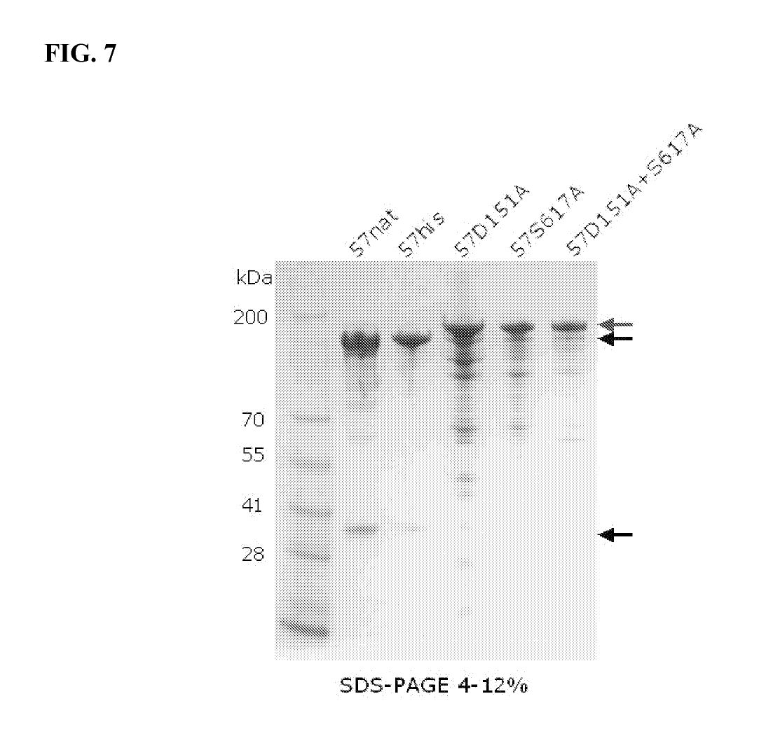

In contrast to wild-type Spy0416, Spy0416 mutants of the invention are obtained as proteins of higher molecular weight (174 kDa), and the 23 kDa band is absent (see FIG. 7, which shows the results of an experiment in which partially purified wild-type Spy0416 and Spy0416 mutants were loaded on SDS-polyacrylamide gels).

### Example 14

### Dose-Dependent Inhibition of Spy0416-Mediated IL-8 Cleavage by Polyclonal Antisera

Mouse antisera specific for Spy0416, wild type and inactive mutants, were produced by immunizing CD1 mice with the purified recombinant proteins.

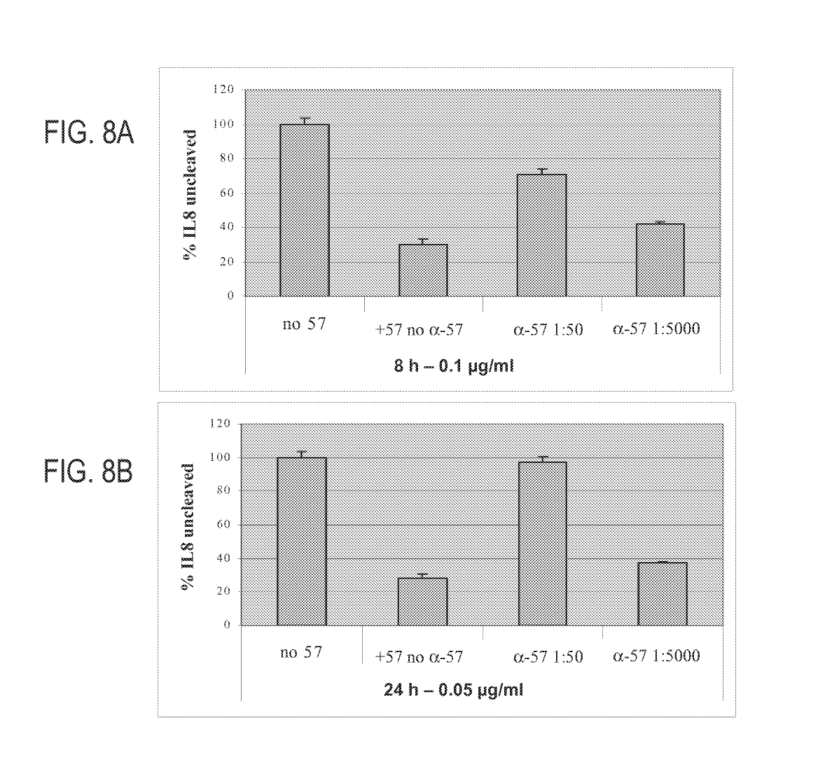

IL-8 (10 μg/ml) was incubated with wild-type Spy0416 with or without Spy0416 antiserum (1:50 and 1:5000) in two different conditions: (1) 8 hour incubation, 0.1 μg/ml of Spy0416 and (2) 24 hour incubation, 0.05 μg/ml of Spy0416. The incubation mixtures were then tested for the presence of uncleaved IL-8 by ELISA. The results shown in FIGS. 8A and 8B demonstrated a dose-dependent inhibition of Spy0416-mediated IL-8 cleavage by the mouse antiserum.

### Example 15

### Cloning of Wild-Type and Mutant Spy0167 Proteins

Genes encoding wild-type and mutant Spy0167 proteins were amplified by PCR using the primers from the SF370 genome shown in Table 4.

The PCR products were digested with NheI-XhoI and ligated with pet24b+(Novagen) vector cut with the same enzymes. E. coli DH5α electrocompetent cells were transformed with the ligation reactions. LBPTK medium was added and, after incubation for 1 h at 37° C., with agitation at 250 rpm, bacteria were plated onto LBPTK plates containing 50 μg/ml kanamycin. Positive colonies were identified by colony PCR.

Plasmids from positive colonies were prepared from an overnight culture in LBPTK medium containing 50 μg/ml kanamycin and analyzed by DNA sequencing, which confirmed the expected insert gene under the T7 polymerase promoter. The final DNA and protein sequences of the cloned genes are shown in the sequence listing. See Table 5.

TABLE 4

| gene | primers |
|---|---|
| Spy0167 wild- | 25F NheI, GTGCGTGCTAGCGAATCGAACAAACAAAACACTGC (SEQ ID NO: 157) |
| type tag-less | 25rev = GCATTCGATCCTCGAGCTACTTATAAGTAATCGAACCATATG |
| | (SEQ ID NO: 158) |
| Spy0167 P427L | External primers: |
| tag-less | 25F NheI, GTGCGTGCTAGCGAATCGAACAAACAAAACACTGC (SEQ ID NO: 157) |
| | 25rev, GCATTCGATCCTCGAGCTACTTATAAGTAATCGAACCATATG (SEQ ID NO: 158) |
| | Internal primers: |
| | PL427_for, GCTACCTTCAGTAGAAAAAACCTAGCTTATCCTATTTCATACACC |
| | (SEQ ID NO: 159) |
| | PL427_rev, GGTGTATGAAATAGGATAAGCTAGGTTTTTTCTACTGAAGGTAGC |
| | (SEQ ID NO: 160) |
| Spy0167 Wild | 25F NheI, GTGCGTGCTAGCGAATCGAACAAACAAAACACTGC (SEQ ID NO: 157) |
| Type His- | 25revhis, GCATTCGATCCTCGAGCTTATAAGTAATCGAACCATATGGG |
| tagged | (SEQ ID NO: 161) |
| Spy0167 | External primers: |
| W535F His- | 25F NheI, GTGCGTGCTAGCGAATCGAACAAACAAAACACTGC (SEQ ID NO: 157) |
| tagged | 25revhis, GCATTCGATCCTCGAGCTTATAAGTAATCGAACCATATGGG |
| | (SEQ ID NO: 161) |
| | Internal primers: |
| | WF535_for, GAGTGCACTGGCTTAGCTTTCGAATGGTGGCGAAAAGTGATC |
| | (SEQ ID NO: 162) |
| | WF535_rev, GATCACTTTTCGCCACCATTCGAAAGCTAAGCCAGTGCACTC |
| | (SEQ ID NO: 163) |
| Spy0167 | External primers: |
| W535F-D482N | 25F NheI, GTGCGTGCTAGCGAATCGAACAAACAAAACACTGC (SEQ ID NO: 157) |
| His-tagged | 25revhis, GCATTCGATCCTCGAGCTTATAAGTAATCGAACCATATGGG |
| | (SEQ ID NO: 161) |
| | Internal primers: |
| | WF535_for, GAGTGCACTGGCTTAGCTTTCGAATGGTGGCGAAAAGTGATC |
| | (SEQ ID NO: 162) |
| | WF535_rev, GATCACTTTTCGCCACCATTCGAAAGCTAAGCCAGTGCACTC |
| | (SEQ ID NO: 163) |
| | and |
| | DN482_for, |
| | GTTGCTCAATATGAAATCCTTTGGAATGAAATCAATTATGATGACAAAGGAAAAG |
| | (SEQ ID NO: 164) |
| | DN482_rev, |
| | CTTTTCCTTTGTCATCATAATTGATTTCATTCCAAAGGATTTCATATTGAGCAAC |
| | (SEQ ID NO: 165) |
| Spy0167 | External primers: |
| C530G His | 25F NheI, GTGCGTGCTAGCGAATCGAACAAACAAAACACTGC (SEQ ID NO: 157) |
| tagged | 25revhis, GCATTCGATCCTCGAGCTTATAAGTAATCGAACCATATGGG |
| | (SEQ ID NO: 161) |
| | Internal primers: |
| | CG530_for, CCGTATCATGGCTAGAGAGGGCACTGGCTTAGCTTGGGAATG |
| | (SEQ ID NO: 166) |
| | CG530_rev, CATTCCCAAGCTAAGCCAGTGCCCTCTCTAGCCATGATACGG |
| | (SEQ ID NO: 167) |
| Spy0167 P427L | External primers: |
| His tagged | 25F NheI, GTGCGTGCTAGCGAATCGAACAAACAAAACACTGC (SEQ ID NO: 157) |
| | 25 revhis, GCATTCGATCCTCGAGCTTATAAGTAATCGAACCATATGGG |
| | (SEQ ID NO: 161) |
| | Internal primers: |
| | PL427for, GCTACCTTCAGTAGAAAAAACCTAGCTTATCCTATTTCATACACC |
| | (SEQ ID NO: 149) |
| | PL427_rev, GGTGTATGAAATAGGATAAGCTAGGTTTTTTCTACTGAAGGTAGC |
| | (SEQ ID NO: 150) |
| Spy0167 | External primers: |
| P427L-W535F- | 25_F, GTGCGTGCTAGCGAATCGAACAAACAAAAC (SEQ ID NO: 168) |
| C535G tag-less | 25_stopR, GCGTCTCGAGTCACTTATAAGTAATCGAACCATA (SEQ ID NO: 17450) |
| | Internal primers: |
| | W-C_for, CCGTATCATGGCTAGAGAGGGCACTGGCTTAGCTTTCGAATG |
| | (SEQ ID NO: 170) |
| | W-C_rev, CATTCGAAAGCTAAGCCAGTGCCCTCTCTAGCCATGATACGG |
| | (SEQ ID NO: 171) |
| Spy0167 | External primers: |
| P427L-W535F | 25_F, GTGCGTGCTAGCGAATCGAACAAACAAAAC (SEQ ID NO: 168) |
| tag-less | 25_stopR, GCGTCTCGAGTCACTTATAAGTAATCGAACCATA (SEQ ID NO: 169) |
| | Internal primers: |
| | WF535_for, GAGTGCACTGGCTTAGCTTTCGAATGGTGGCGAAAAGTGATC |
| | (SEQ ID NO: 162) |
| | WF535_rev, GATCACTTTTCGCCACCATTCGAAAGCTAAGCCAGTGCACTC |
| | (SEQ ID NO: 163) |
| Spy0167 | External primers: |
| P427L-0530G | 25_F, GTGCGTGCTAGCGAATCGAACAAACAAAAC (SEQ ID NO: 168) |
| tag less | 25_stopR, GCGTCTCGAGTCACTTATAAGTAATCGAACCATA (SEQ ID NO: 169) |
| | Internal primers: |
| | CG530_for, CCGTATCATGGCTAGAGAGGGCACTGGCTTAGCTTGGGAATG |
| | (SEQ ID NO: 166) |
| | CG530_rev, CATTCCCAAGCTAAGCCAGTGCCCTCTCTAGCCATGATACGG |
| | (SEQ ID NO: 167) |
| Spy0167 | External primers: |
| A248 his- | 25F NheI, GTGCGTGCTAGCGAATCGAACAAACAAAACACTGC (SEQ ID NO: 157) |
| tagged | 25 revhis, GCATTCGATCCTCGAGCTTATAAGTAATCGAACCATATGGG |
| | (SEQ ID NO: 161) |
| | Internal primers: |
| | Δ248for, CTGGTGGTAATACGCTTCCTAGAACACAATATACTGAATCAATGG |
| | (SEQ ID NO: 172) |
| | Δ248rev, CCATTGATTCAGTATATTGTGTTCTAGGAAGCGTATTACCACCAG |
| | (SEQ ID NO: 173) |

TABLE 5

| | sequence identifier | | | |
|---|---|---|---|---|
| | amino acid | | nucleotide | |
| Spy0167 gene | tag-less | His-tagged | tag-less | His-tagged |
| wild-type | 1-12 | 13 | 28 | 14 |
| P427L | 20 | 15 | 29 | 57 |
| C530G | 22 | 16 | 31 | 58 |
| W535F | 21 | 18 | 30 | 52 |
| ΔA248 | 23 | 17 | | 59 |
| W535F + D482N | 24 | 19 | | 53 |
| P427L + C530G | 26 | 54 | 33 | |
| P427L + W535F | 25 | 55 | 32 | |
| P427L + C530G + W535F | 27 | 56 | 34 | |

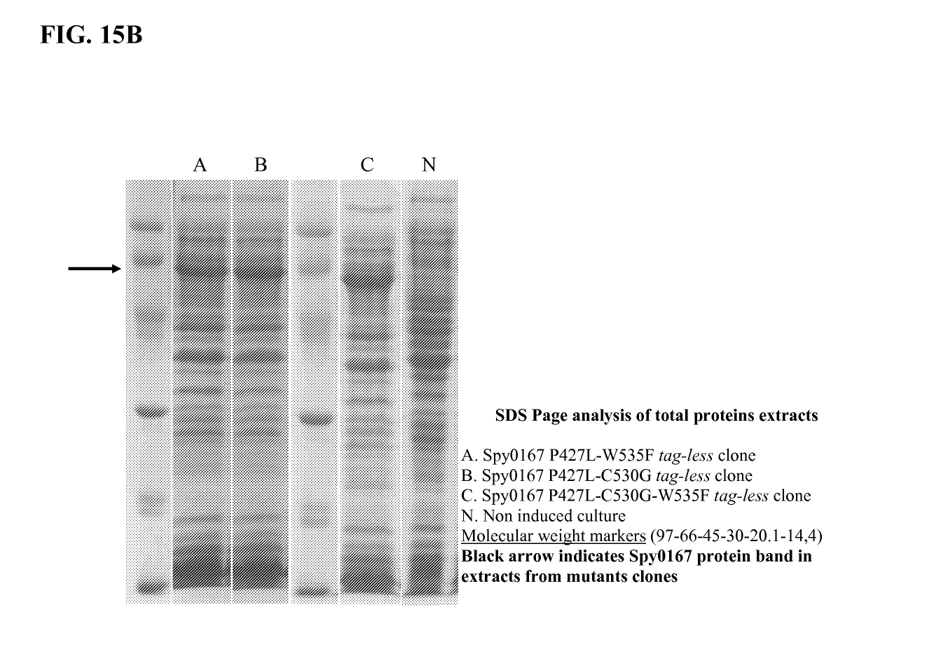

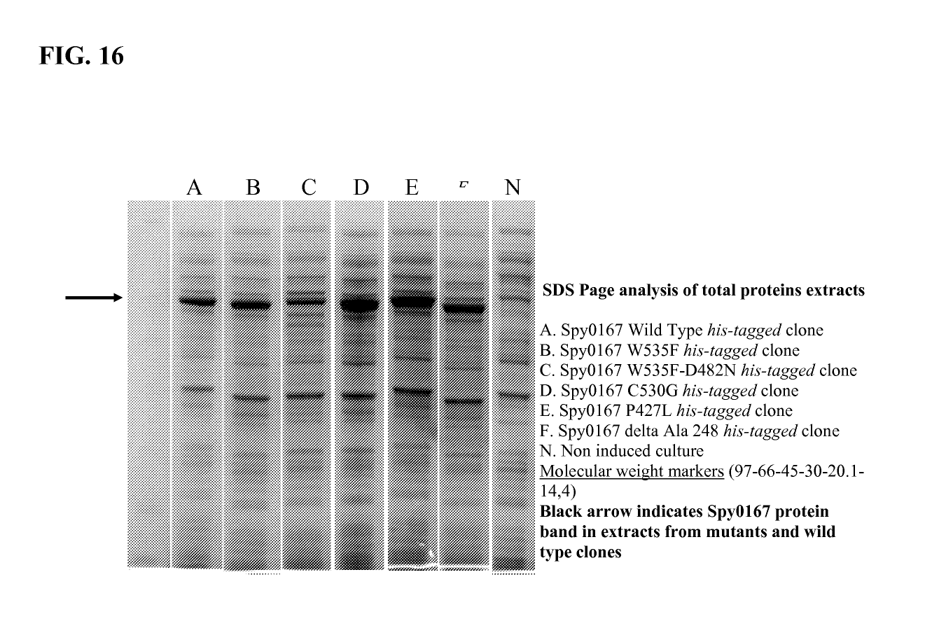

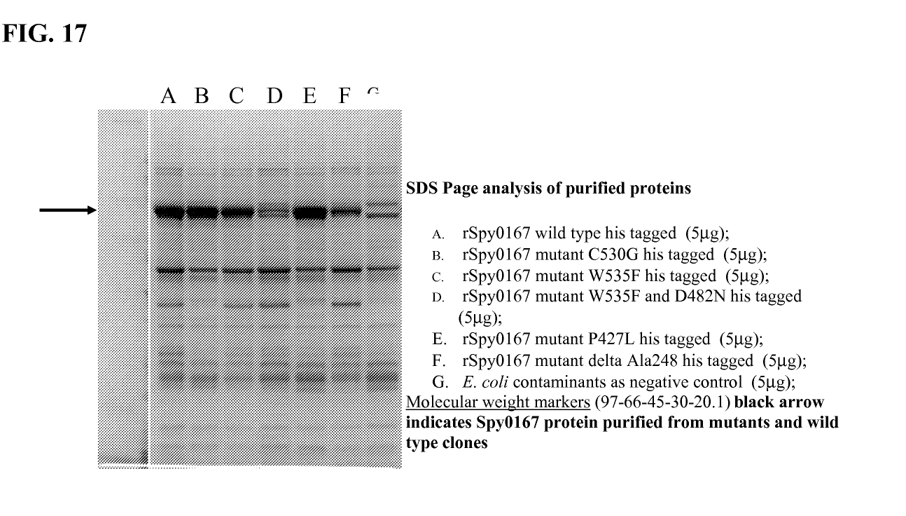

E. coli BL21(DE3) (Novagen) competent cells were transformed with the correct construct. LBPTK medium was added and, after incubation for 1 h at 37° C., with agitation at 250 rpm, bacteria were plated onto LBPTK plates containing 50 μg/ml kanamycin. BL21(DE3) pet24b+Spy0167 wild-type tag-less cells were grown at 25° C. and induced with 1 mM IPTG. Clone expression was verified by SDS PAGE (tag-less, FIGS. 15A and 15B; His-tagged, FIG. 16).

### Example 16

### Purification of His-Tagged Proteins

E. coli pellets were suspended in lysis buffer and mixed for 30-40 minutes at room temperature. Lysates were centrifuged at 30-40000×g for 20-25 minutes and supernatants were loaded onto wash buffer A equilibrated columns (Poly-Prep with 1 ml of Ni-Activated Chelating Sepharose Fast Flow resin). The loaded resin was washed three times with wash buffer A and three times with wash buffer B. Proteins were eluted with elution buffer in Eppendorf tubes containing 2 mM final of DTT. Total elution proteins are quantified with Bradford reagent and then analyzed by SDS-polyacrylamide gel electrophoresis (FIGS. 15 and 16).

Buffers

### Lysis Buffer:

10 ml B-PER™ (Bacterial-Protein Extraction Reagent, Pierce cat. 78266)
MgCl₂ final concentration of 0.1 mM
DNAsi I (Sigma cat. D-4263) 100 units
lysozyme (Sigma cat. L-7651) final concentration of 1 mg/ml
   wash buffer A: 50 mM NaH₂PO₄, 300 mM NaCl, pH 8.0
   wash buffer B: 20 mM imidazole, 50 mM NaH₂PO₄, 300 mM NaCl, pH 8.0
   elution buffer: 250 mM imidazole, 50 mM NaH₂PO₄, 300 mM NaCl, pH 8.0

### Example 17

### Purification of Tag-Less Proteins

Lysate Preparation

About 80-110 g of bacterial culture pellet were suspended in 200-280 ml B-PER™ reagent (Pierce) supplemented with 6 tablets of COMPLETE® protease inhibitor, 10 ml 0.2M EDTA pH 7.5 (5 mM final concentration), 10 ml of a 100 mg/ml lysozyme solution, 8 ml of a 10000 K units/ml DNAse I solution and 1 ml of 50 mM MgCl₂ solution. Bacterial lysis was achieved by shaking the bacterial suspension for 60 minutes until a homogeneous suspension was obtained.

Following centrifugation for 60 minutes at 13000 rpm (25400×g), the supernatant was filtered using a 0.22 μm filter and is diluted with H₂O until a 1.8-1.9 mS conductivity was obtained. The pH was adjusted to 8.0. Protein concentration was determined by the Bradford method.

Anionic Exchange Chromatography

The supernatant derived from the lysate treated as described above was loaded on an HP 50/10 Q Sepharose column (˜200 ml), previously equilibrated with 30 mM TRIS, pH 8.0. The flow-through was collected. Fractions containing the Spy0167 protein were pooled and dialyzed against 10 mM Na phosphate, pH 6.8. Protein concentration was determined by the Bradford method.
Buffer A: 30 mM TRIS, pH 8.0
Buffer B: 30 mM TRIS, 1M NaCl, pH 8.0
Equilibrium and Loading: 0% B
Gradient: 0-25% B in 5 CV-25% B 2 CV
Wash: 100% B 2 CV+3 CV
Flux: 20 ml/min
Fraction volume: 14 ml

Hydroxylapatite Chromatography

The previously obtained pool was loaded on a CHT20 column previously equilibrated with 10 mM Na-phosphate, pH 6.8. The flow through was collected.
Buffer A: 10 mM Na-phosphate, pH 6.8
Buffer B: 500 mM Na phosphate, pH 6.8
Wash: 8 CV
Wash: 30% B 6 CV
Gradient: 30-100% B (10 CV)
Wash: 100% B
Flux: 5 ml/min.
Fraction volume: 5 ml

Fraction aliquots were loaded on 12% Criterion gels under reducing and non-reducing conditions. Fractions containing Spy0167 protein were pooled and protein concentration was determined by Bradford method.

Gel Filtration Chromatography

The collected pool was concentrated using an Amicon filter in order to get a volume <10 ml. The concentrated material was loaded on a HiLoad Superdex 200 26/60 equilibrated with at least 3-4 column volumes of PBS.
Buffer: PBS
Elution: Isocratic
Flux: 2.5 ml/min.
Fraction volume: 5 ml

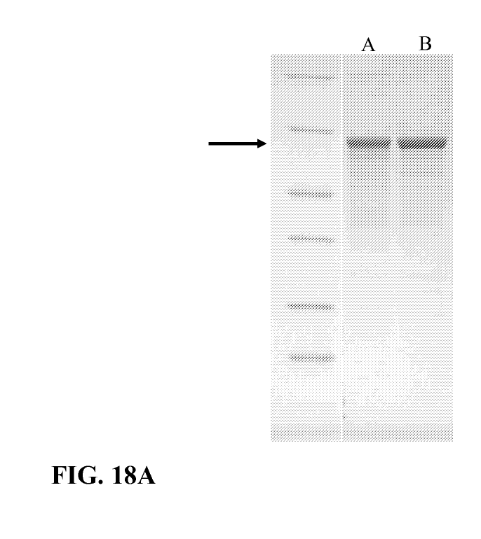

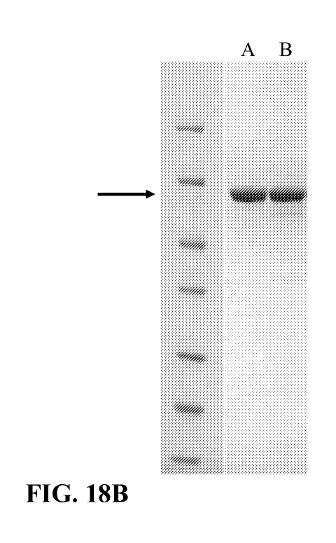

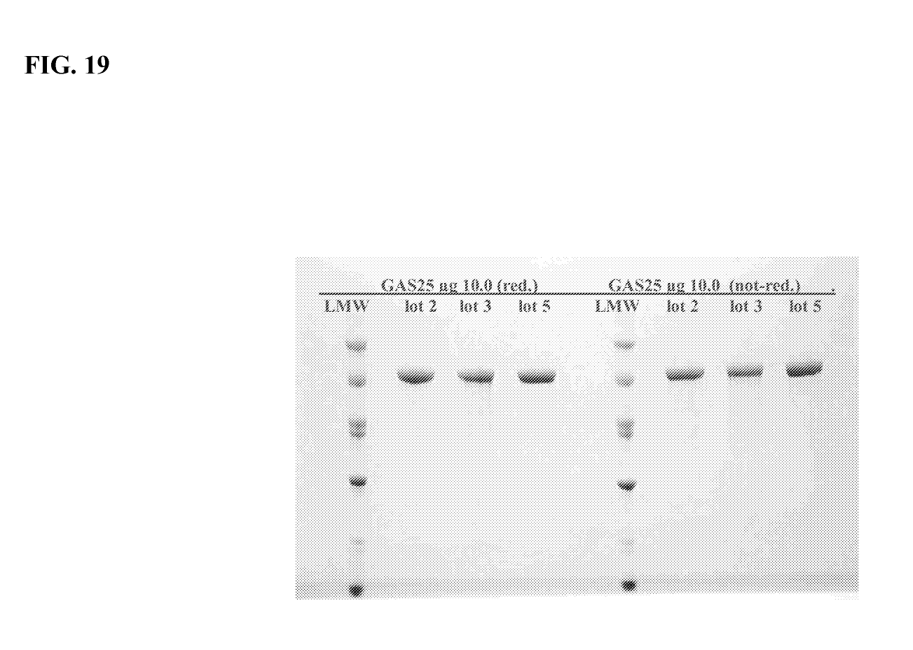

Fractions containing Spy0167 protein were pooled and protein concentration was determined by Bradford. An additional estimation of protein concentration was performed by UV measurement considering Abs 0.1% (=1 g/l) 1.119. Protein purity is analyzed by polyacrylamide gel electrophoresis (FIG. 18).

### Example 18

### Hemolytic Assays

Protocol for Quantitative Hemolytic Assay

Serial dilutions of toxin were prepared in 96-well plates with U-shaped bottoms using PBS+0.5% BSA. One ml of sheep blood was washed three times in PBS (with centrifugation at 3000×g), and blood cells were suspended in 5 ml of PBS. An equal volume of suspension was added to 50 μl of each toxin dilution and incubated at 37° C. for 30 min. Triton (2%) in water was used to give 100% hemolysis, and PBS+0.5% BSA was used as negative control. Plates were then centrifuged for 5 min at 1,000×g, and the supernatant was transferred carefully to 96-well flat-bottomed plates. The absorbance was read at 540 nm.

Comparison of E. coli Extracts Containing Wild-Type Spy0167 and Spy0167 Mutant P427L

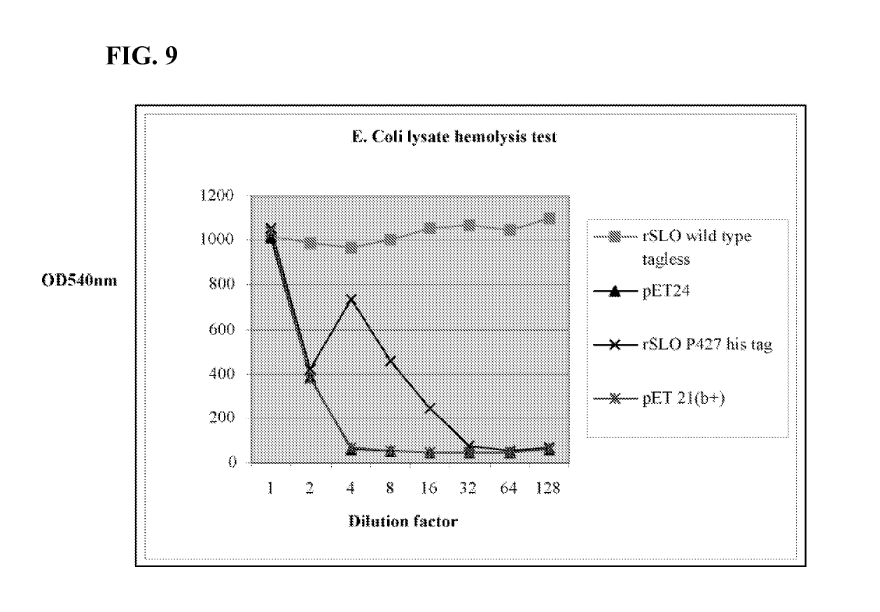

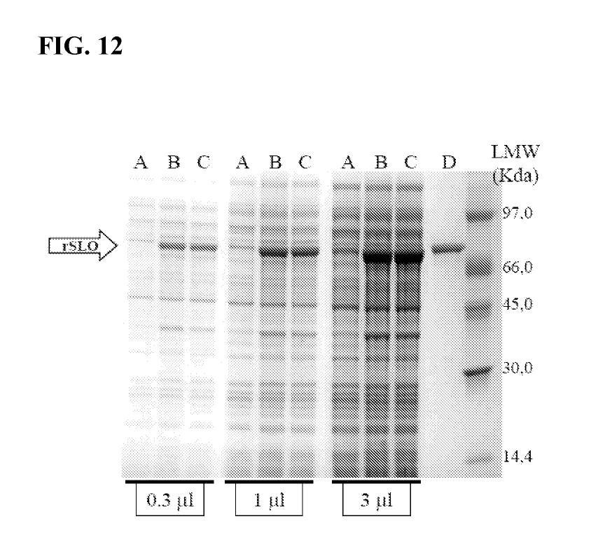

The gene encoding Spy0167 P427L was amplified using PCR from the SF370 M1 genome and cloned into the vector pET21b+, which allowed expression in E. coli BL21DE3 of the His-tagged protein. Soluble extracts of E. coli expressing similar amounts of the wild-type and mutated streptolysin O proteins (see FIG. 12) were used to perform a hemolytic assay to compare the cytolytic properties of the two antigens. The result of the assay is shown in FIG. 9, which demonstrates that the mutated protein is at least 100 times less toxic than wild-type.

Comparison of Purified Wild-Type Spy0167 and Spy0167 Mutant P427L

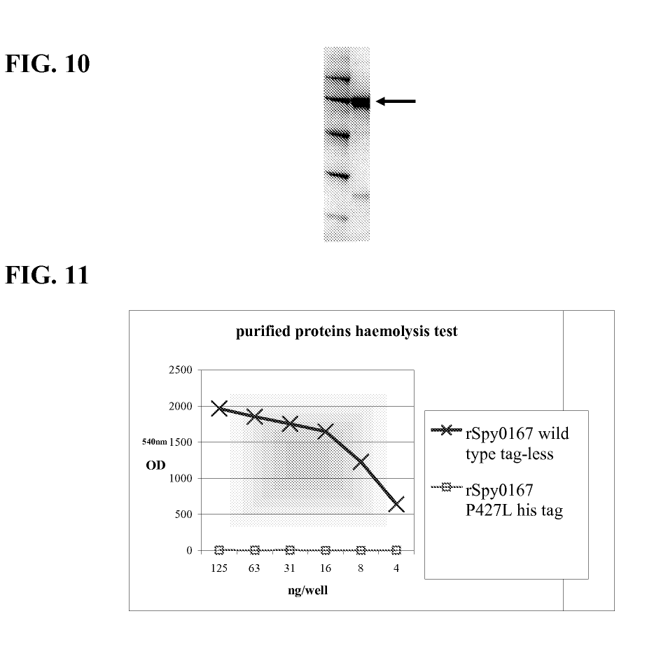

The Spy0167 P427L mutant was purified according to purification standard procedures for His-tagged recombinant proteins (FIG. 10). Different concentrations of the purified wt and mutated proteins were used to repeat the hemolytic assay, which confirmed the decreased cytolytic activity (FIG. 11).

Hemolytic Activity of E. coli Extracts Containing His-Tagged and Tag-Less Wild-Type Spy0167 and Spy0167 Mutant P427L

We compared the hemolytic activity of E. coli lysates transformed with wild-type recombinant Spy0167 (rSpy0167) without a His tag (BL21 DE3, Novagen No. 71382-pET24) and P427L mutant rSpy0167 without a His tag (BL21 DE3, Novagen No. 71382-pET24). E. coli BL21 DE3 (Novagen, No. 71382) transformed with pET24 without insert was used as a negative control. The positive control was a hypotonic solution containing Triton 2% in water. The negative control was the protein dilution buffer (PBS containing 0.5% BSA, pH 7.4).

Hemolysis was determined by measuring absorbance at 540 nm (A₅₄₀ₙₘ) of the supernatants. The titer was calculated as the dilution with 50% of maximum A₅₄₀ₙₘ.

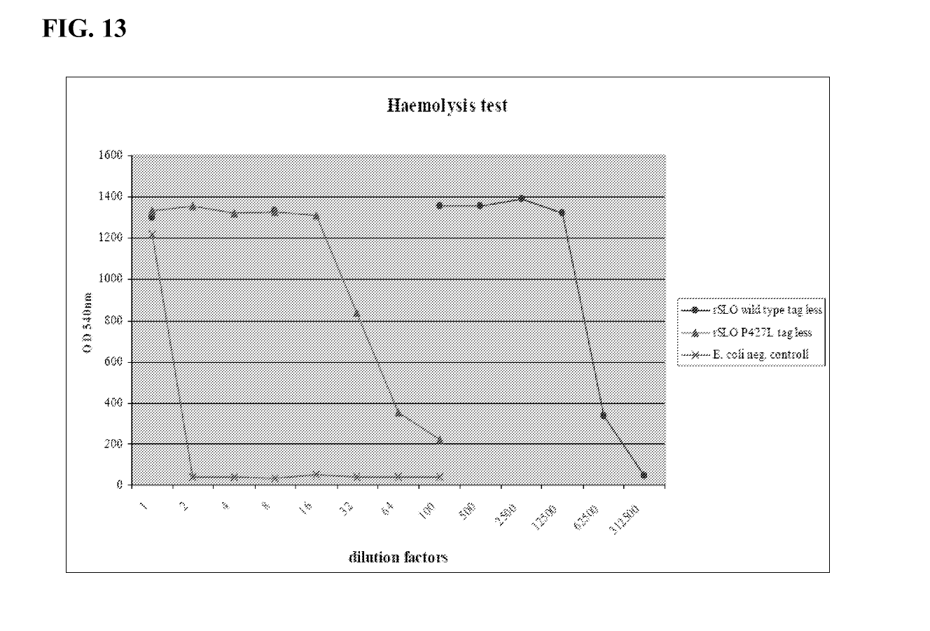

Results are shown in Tables 6 and 7 and in FIG. 13. These data demonstrate that, under the same conditions, mutant P427L is 1000 times less hemolytic than wild type Spy0167.

TABLE 6

| E. coli | CFU/ml |
|---|---|
| negative control | 3.9 × 10⁸ |
| Wild-type rSpy0167 (tag-less} | 1.2 × 10⁹ |
| P427L rSpy0167 (tag-less) | 1.03 × 10⁹ |

TABLE 7

| | rSpy0167 wild-type | rSpy0167P427L |
|---|---|---|
| | tag-less | tag-less |
| titer (OD = 50% hemolysis) | 50,000 | 48 |
| titer Wt/P427L | 1042 | |

Comparison of Wild-Type Spy0167 and Various Spy0167 Mutants

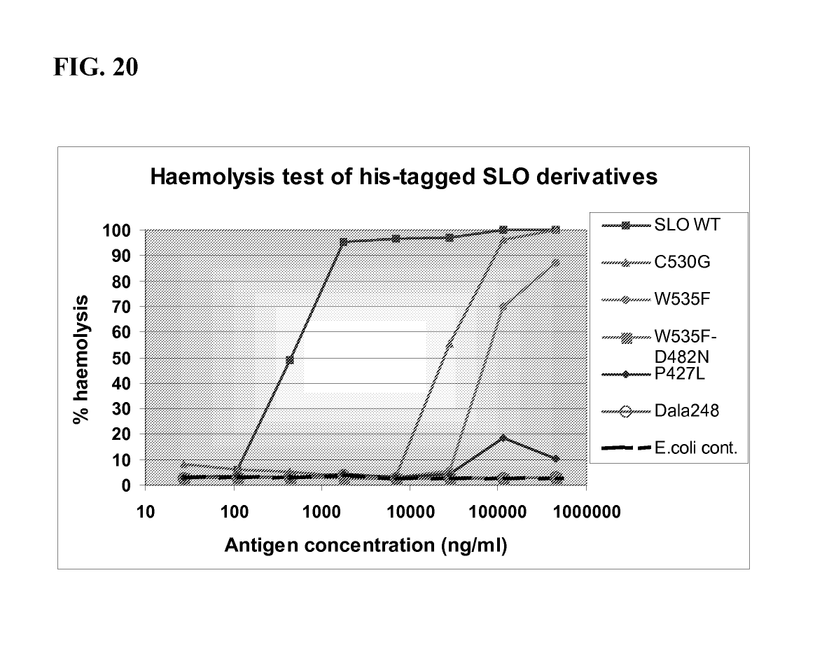

Hemolytic activity of wild-type Spy0167 was compared with hemolytic activity of several different Spy0167 mutants. The results are shown in FIG. 20 and in Table 8, below. One hemolytic unit (HU) is defined as the amount of toxin required to obtained 50% of maximum lysis obtained treating the blood cells with 2% Triton.

TABLE 8

| Protein | HU/mg | HU/mg-Spy0167/mutants |
|---|---|---|
| rSpy0167 WT | 22760 | 1 |
| C530G | 620 | 37 |
| W535F | 160 | 146 |
| W535F-D482N | <<20 | >>1000 |
| P427L | about 20 | about 1000 |
| Δala248 | <<20 | >>1000 |
| Neg. Control | <<20 | >>1000 |

Due to differences in protein purity, the hemolysis units/mg of mutants indicated in bold are overestimated; however, it is clear that (1) mutant W535F is less hemolytic than mutant C530G; (2) mutant P427L is about 1000 times less hemolytic than wild type and about 6-25 times less hemolytic than other two mutants W535F and C530G; and (3) mutant Δ248 is certainly less hemolytic than wild type).

Effect of Cholesterol

Two-fivefold serial dilutions in PBS-BSA 0.5% of E. coli lysates or E. coli lysate with 200 mg/ml of cholesterol obtained after cells' growing at 30° C. and induction with 1 mM IPTG at 25° C. and OD₆₀₀ₙₘ about 0.4-0.6, were assayed for their hemolytic activity. Fifty microliters of a 2% sheep erythrocyte solution in PBS were treated with an equal volume of protein preparations obtained by lysing bacteria, 3 hours after induction, with lysis buffer (B-PER solution-PIERCE-1 mM MgCl₂, 100K units/ml DNAse (Sigma) and lysozyme (Sigma) for 30-40 minutes. The insoluble fraction was then centrifuged (15 minutes, 21000×g, 4° C.), and the supernatant (E. coli lysate) was transferred to a new Eppendorf tube containing DTT at final concentration of 5 mM.

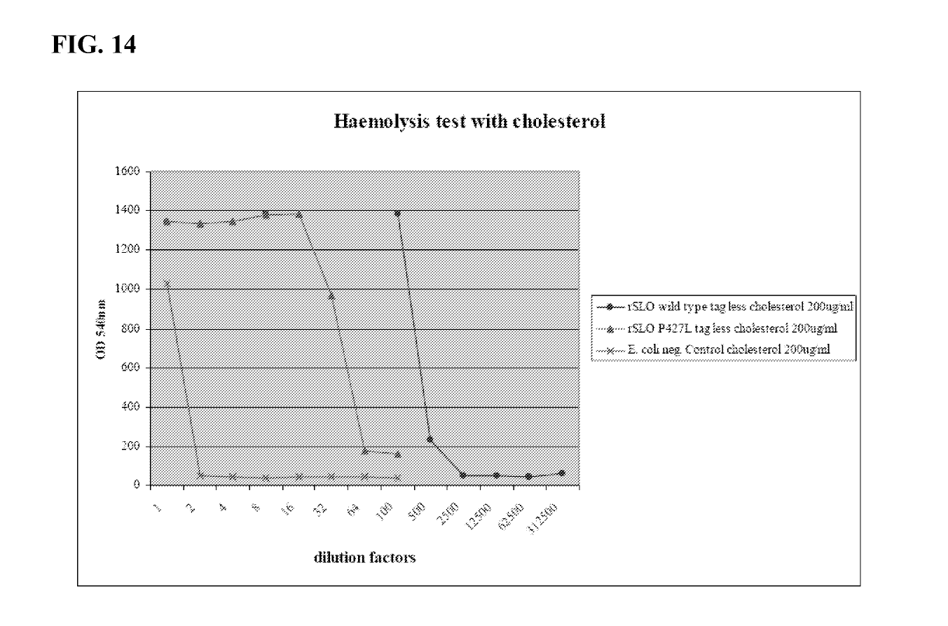

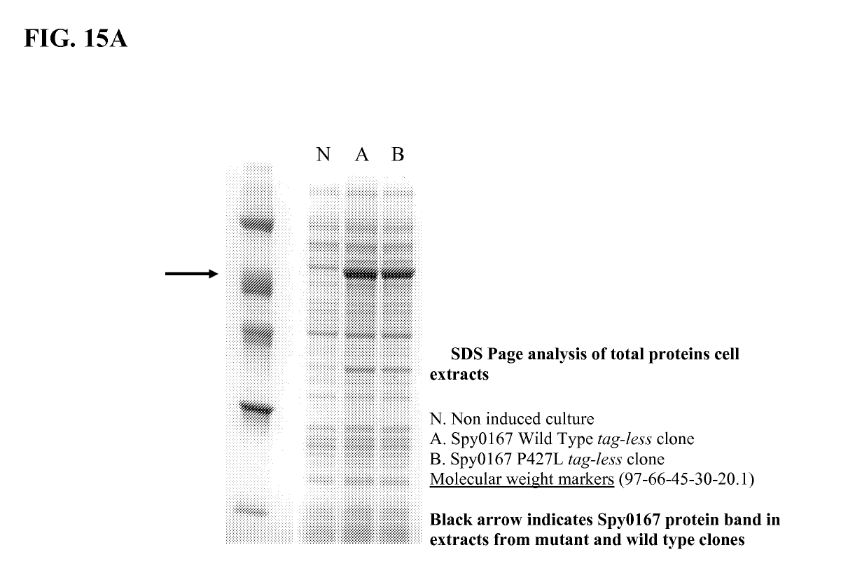

Under this condition, cholesterol did not inhibit either wild-type or mutant Spy0167 until a 100-fold dilution factor was used; thus, there was no effect on the mutant-induced lysis. In contrast, wild-type-induced lysis was greatly reduced. Lysis induced by the negative control was not influenced by cholesterol, which suggests that cholesterol-induced inhibition is specific. See Table 9 and FIG. 14.

TABLE 9

| | rSpy0167 wild-type | rSpy0167 P427L |
|---|---|---|
| | tag-less | tag-less |
| titer (OD = 50% hemolysis) | 400 | 40 |
| titre Wt/P427L | 10 | |

### Example 19

### Inhibition of Hemolysis

Protocol

Serial two-fold dilutions of sera from mice immunized with wild-type or mutant Spy0167 proteins (without adjuvants or with Alum or MF59™ as adjuvants) were prepared in 96-well plates with U-shaped bottoms using PBS+0.5% BSA. Sera of mice immunized with PBS or with adjuvant alone, as appropriate, were used as negative controls. An equal volume of a 50-100 ng/ml (3.5-7 HU) toxin solution in PBS+0.5% BSA was added, and the plates were incubated at room temperature for 20 minutes under agitation (800 rpm). After incubation, 50 ml of this solution were transferred to a new 96-well plate, and an equal volume of a sheep red blood cell suspension (washed 3× in PBS) was added and incubated at 37° C. for 30 min. Plates were then centrifuged for 1 min at 1,000×g, the supernatant was carefully transferred to 96-well flat-bottomed plates, and the absorbance was read at 540 nm. In the results described below, inhibition titer is expressed as the sera dilution that reduced Triton-induced hemolysis by 50%.

Inhibition of Spy0167 Hemolysis by Wild-Type Spy0167 Antisera

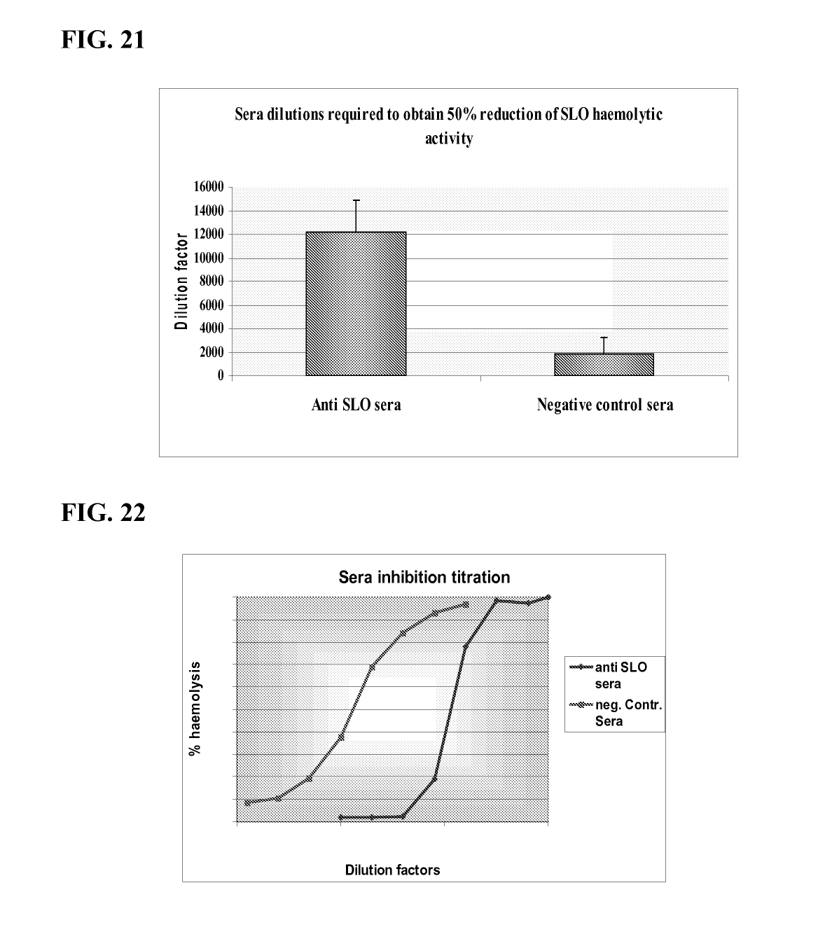

Inhibition of Spy0167 hemolysis by anti-wild-type Spy0167 antisera is shown in FIGS. 21-23 and Tables 10-12. Anti-Spy0167 sera titers are included between 1/7,000 and 1/14,000 (arithmetic mean, 1/12,167±2,714. Negative control sera (Freund's adjuvant) titers are included between 1/375 and 1/4,000 (arithmetic mean, 1/1,854±1,384).

TABLE 10
(shown graphically in FIG. 22).
arithmetic mean of tested sera - % hemolysis

| | anti- | negative |
|---|---|---|
| dilution | Spy0167 | control |
| factor/sera | sera | sera |
| 125 | | 9 |
| 250 | | 10 |
| 500 | | 19 |
| 1,000 | 2 | 38 |
| 2,000 | 2 | 69 |
| 4,000 | 2 | 84 |
| 8,000 | 19 | 93 |
| 16,000 | 78 | 97 |
| 32,000 | 99 | |
| 64,000 | 97 | |
| 128,000 | 100 | |

TABLE 11

| | |
|---|---|
| anti-Spy0167 sera | negative control sera |
| (Freund's adjuvant) | (Freund's adjuvant) |

| | 50% hemolysis | | 50% hemolysis |
|---|---|---|---|
| serum | inhib. | serum | inhib. |
| A | 14,000 | 1 | 4,000 |
| B | 7,000 | 2 | 1,500 |
| C | 12,000 | 3 | 375 |
| D | 12,000 | 4 | 3,000 |
| E | 14,000 | 5 | 1,500 |
| F | 14,000 | 6 | 750 |

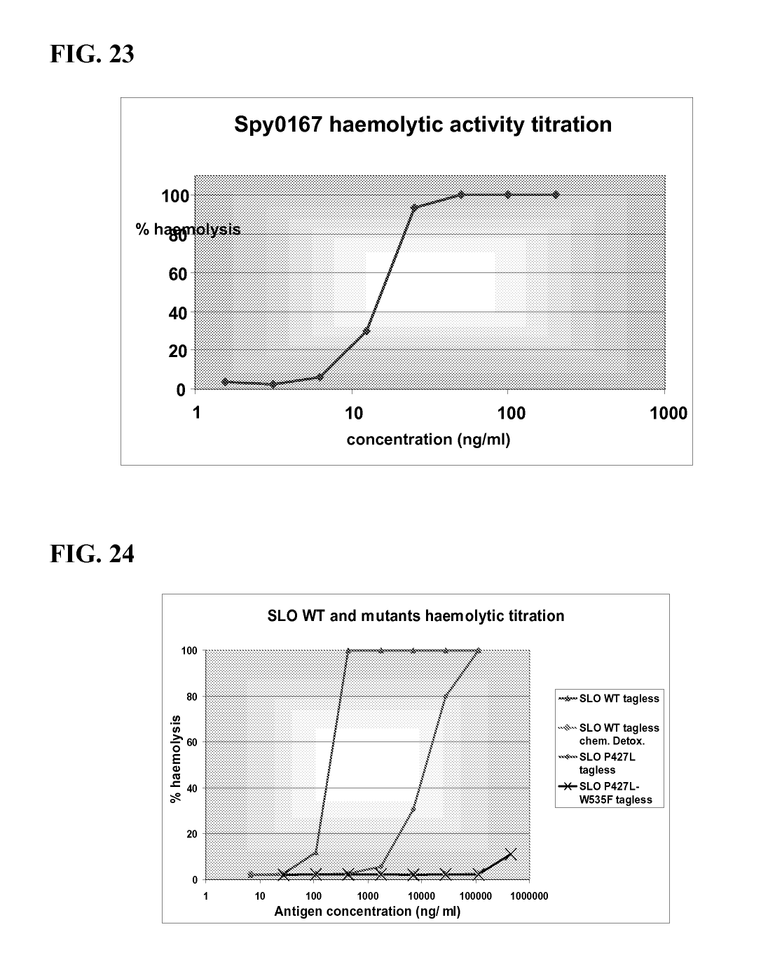

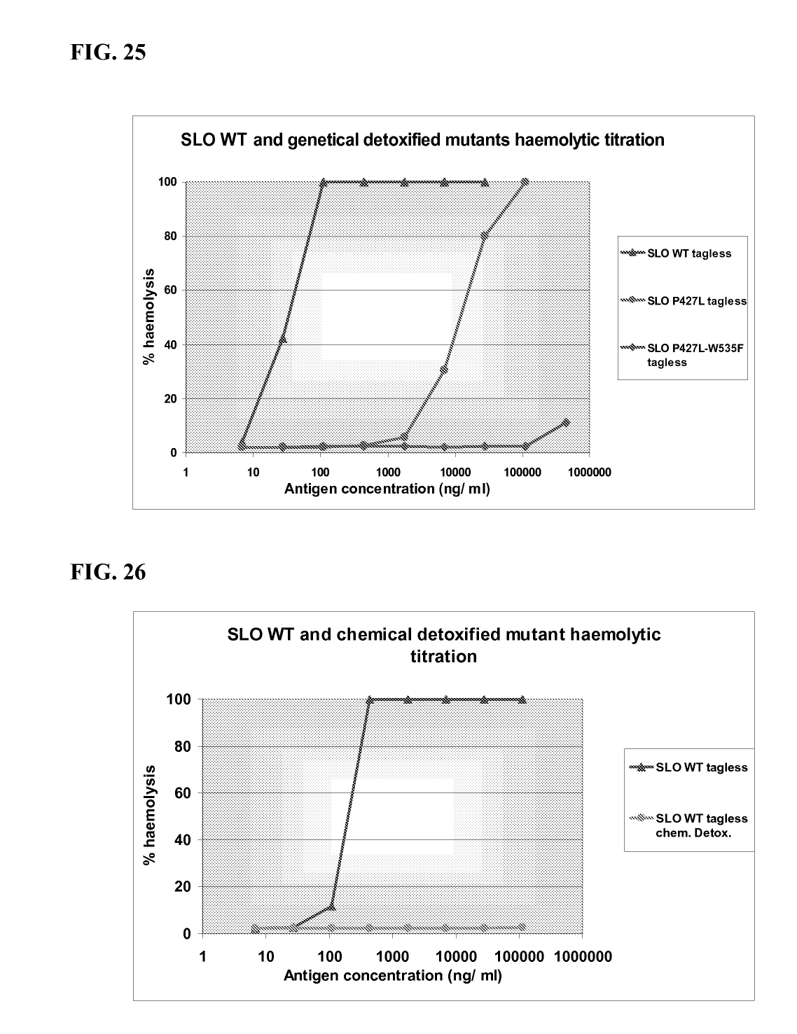

TABLE 12
(shown graphically in FIG. 23)

| ng/ml Spy0167 | % hemolysis |
|---|---|
| 1.6 | 4 |
| 3.1 | 3 |
| 6.3 | 6 |
| 12.5 | 30 |
| 25 | 94 |
| 50 | 100 |
| 100 | 100 |
| 200 | 100 |

Titration of Hemolytic Activity of Wild-Type Spy0167, Chemically Detoxified Wild-Type Spy0167 and Spy0167 Mutants

Titration of hemolytic activity of wild-type Spy0167, chemically detoxified wild-type Spy0167, and Spy0167 mutants (P427L; P427L+W535F) is shown in Table 13.

TABLE 13

| protein | HU/mg | HU/mg-Spy0167/mutants |
|---|---|---|
| Spy0167 wild-type | 728,307 | 1 |
| tag-less | | |
| Spy0167 P427L tag-less | 711 | 1,024 |
| Spy0167 P427L + | <22 (stim. 10) | >33.000 |
| W535F tag-less | | |
| Spy0167 wild-type | 45,511 | |
| tag-less | | |
| Spy0167 wild-type | <<89 | >>511 |
| tag-less, detoxified | | |

Inhibition of Spy0167 Hemolysis by Antiserum Against Mutant Spy0167 Proteins

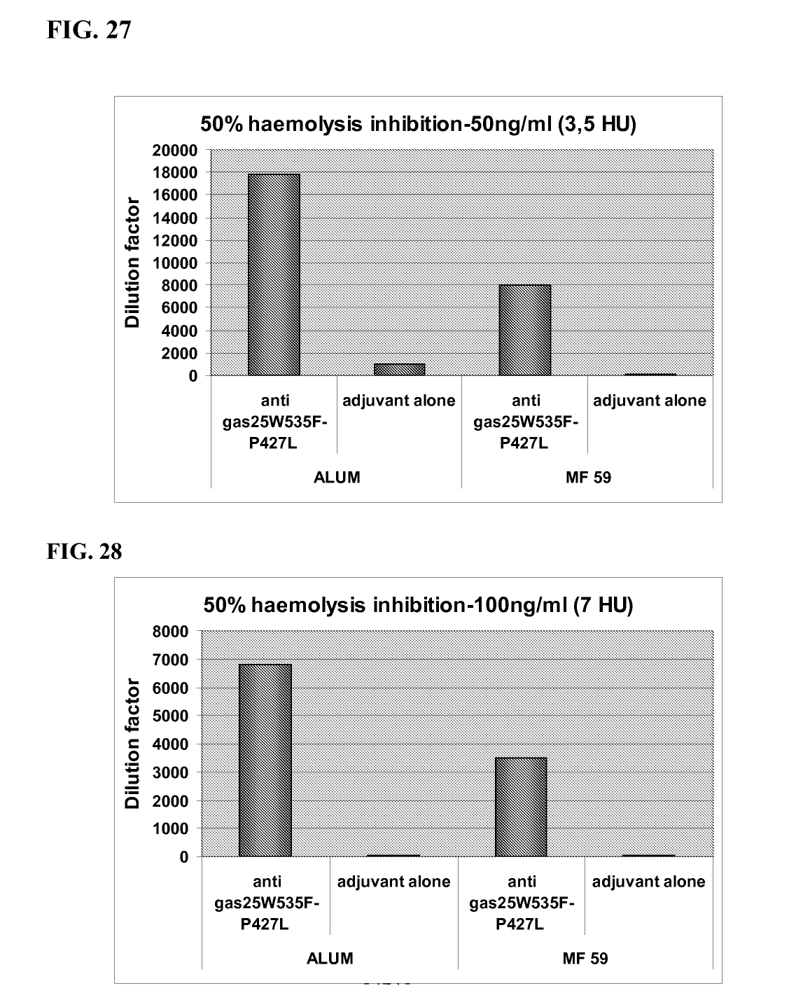

Inhibition of Spy0167 hemolysis by antisera against mutant Spy0167 proteins is shown in FIGS. 27-29 and Tables 14-16. Using 50 ng/ml (3.5 HU) of toxin, the sera dilution required to obtain 50% reduction of Spy0167 hemolytic activity for Spy0167 mutant W535-P427L is 1/17,860 using Alum adjuvant and 1/7991 using MF59™ adjuvant. Negative control (adjuvant alone) titers are 1/1,000 (Alum) and 1/125 (MF59™).

TABLE 14
(shown graphically in FIG.27).
50 ng/ml (3.5 HU) of wild-type Spy0167

| | specific inhibition/ |
|---|---|
| adjuvant | non-specific inhibition |
| alum | 18 |
| MF ™59 | 64 |

TABLE 15
(shown graphically in FIG. 28)
100 ng/ml (37 HU) of wild-type Spy0167

| adjuvant | specific inhibition/non-specific inhibition |
|---|---|
| alum | >227 |
| MF ™59 | >117 |

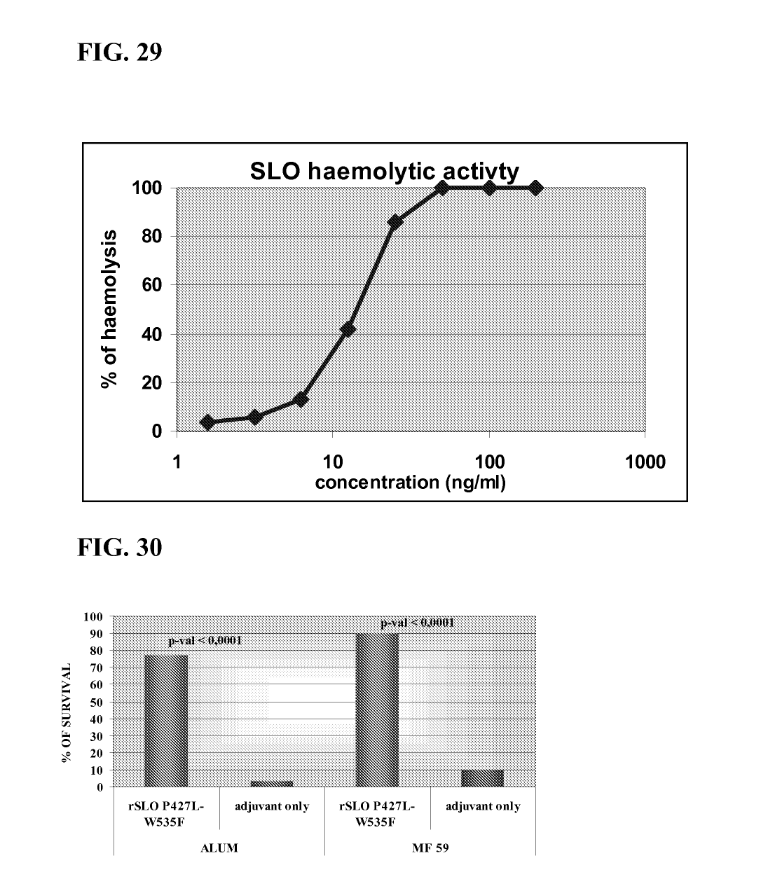

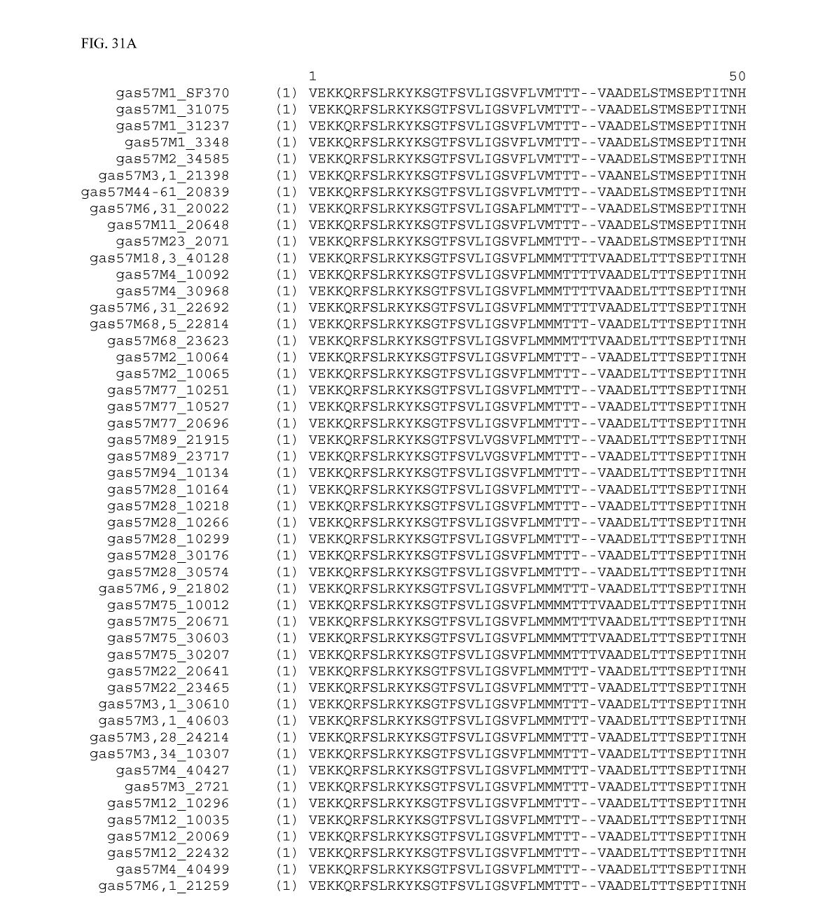

TABLE 16
(shown graphically in FIG. 29)

| ng/ml Spy0167 | % hemolysis |
|---|---|
| 1.6 | 3.5 |
| 3.1 | 5.8 |
| 6.3 | 13 |
| 12.5 | 42 |
| 25 | 86 |
| 50 | 100 |
| 100 | 100 |
| 200 | 100 |

### Example 20

### In Vivo Protection Experiments

The purified Spy0167 P427L protein, together with Freund's adjuvant, was administered intraperitoneally to 40 mice. The mice were then challenged intranasally with the 3348 M1 GAS strain. Table 17 reports the data obtained in 3 separate experiments, showing that 100% protection was consistently achieved in all experiments.

TABLE 17
Infection survival rate of mice

| | % surviving mice | | |
|---|---|---|---|
| antigen | Experiment 1 | Experiment 2 | Experiment 3 |
| Spy0167 Pro247Leu | 100 | 100 | 100 |
| E. coli contaminants | 10 | 10 | 10 |
| (negative control) | | | |
| homologous M1 | 100 | 90 | 90 |
| protein | | | |
| (positive control) | | | |

Groups of 10-20 mice were immunized with 20 μg of the recombinant protein at days 0, 21 and 35. Mice of negative control groups were immunized either with GST alone or with E. coli contaminants, depending on the version of the GAS recombinant protein used. Two weeks after the third immunization, blood samples were taken. A few days afterwards, immunized mice were challenged intranasally with 10⁸ cfu (50 μl) of the M1 3348 GAS strains. Survival of mice was monitored for a 10-14 day period. Immune sera obtained from the different groups were tested for immunogenicity on the entire Spy0167 recombinant protein (western blot analysis). The results are shown in Tables 18 and 19.

TABLE 18

| | | | % negative control |
|---|---|---|---|
| Protein | # mice | % survival | survival |
| Spy0167_Pro247Leu His | 10 | 90 | 30 |
| Spy0167_Pro247Leu His | 10 | 100 | 20 |
| Spy0167_Pro247Leu His | 10 | 80 | 30 |
| Spy0167_WT | 20 | 95 | 15 |
| Spy0167_WT | 10 | 100 | 40 |

TABLE 19

| | | | % negative control |
|---|---|---|---|
| Protein | # mice | % survival | survival |
| rSpy0167 WT his-tagged | 20 | 100 | 45 |
| C530G his-tagged | 20 | 100 | 45 |
| W535F his-tagged | 20 | 100 | 45 |
| W535F-D482N his-tagged | 20 | 100 | 45 |
| P427L his-tagged | 20 | 95 | 45 |
| Δala248 his-tagged | 20 | 100 | 45 |

### Example 21

### In Vivo Toxicity Experiments

Protocols

Intravenous Injection of Spy0167.

A solution of either wild-type or mutant Spy0167 in PBS is diluted in a solution of PBS+2 mM DTT, then 100 ml is injected into the tail vein of a mouse. Mice are observed for 2-3 days. Injection of wild-type Spy0167 typically results in death within a few minutes.

In Vivo Lethality Inhibition Assay.

For lethality inhibition mediated by immune sera, 10 μg/mouse of wild-type Spy0167 (a solution of 100 μg/ml in PBS, 2 mM DTT) are incubated for 20 minutes with rotation at room temperature with either anti-Spy0167 serum or control serum (obtained from mice immunized with adjuvant alone). After incubation, the samples are inoculated in the mice by intravenous injection into the tail vein. Mice are observed for 2-3 days.

The results for wild-type Spy0167 and mutant Spy0167 P427L-W535F are shown in Table 20.

TABLE 20

| | |
|---|---|
| wild-type Spy0167 | P427L-W535F |

| μg/mouse | dead/treated | μg/mouse | dead/treated |
|---|---|---|---|
| | | 100 | 0/4 |
| 50 | 4/4 | 50 | 0/4 |
| 10 | 8/8 | 10 | 0/8 |
| 2 | 0/4 | | |
| 0.4 | 0/4 | | |
| 0.04 | 0/4 | | |

Acute in vivo acute toxicity was assessed using a dose of 10 μg/mouse of wild-type Spy0167 as a positive control and injection of Freund's adjuvant alone as a negative control. Ten μg/mouse of wild-type Spy0167 was incubated with either wild-type Spy0167 antiserum or with control serum and inoculated into mice as described above. The results are shown in Table 21.

TABLE 21
wild-type Spy0167 (10 μg/mouse)

| sera | serum dilution | dead/treated |
|---|---|---|
| none | | 8/8 |
| wild-type | 1/5 | 0/4 |
| Spy0167 | | |
| wild-type | 1/10 | 0/4 |
| Spy0167 | | |
| wild-type | 1/20 | 4/4 |
| Spy0167 | | |
| wild-type | 1/50 | 4/4 |
| Spy0167 | | |
| wild-type | 1/100 | 4/4 |
| Spy0167 | | |
| negative control | 1/5 | 4/4 |

The results of another set of experiments performed as described above are shown in Tables 22 and 23. In vivo acute toxicity was assessed using either 5 or 10 μg/mouse of wild-type Spy0167. In particular, 10 μg/mouse of wild type Spy0167 were preincubated either with sera from mice immunized with Spy0167 P427L-W535F or only PBS (no serum). In addition, 5 μg/mouse of wild type Spy0167 were preincubated either with sera from mice immunized with Spy0167 P427L-W535F or sera from mice immunized with PBS plus adjuvant (Alum), as negative control serum.

The results demonstrate that lethal doses of wild-type Spy0167 are neutralized by anti-Spy0167 P427L-W535F sera but not by negative control sera at the same dilution.

TABLE 22
wild-type Spy0167 (10 μg/mouse)

| Sera | serum dilution | dead/treated |
|---|---|---|
| none | — | 4/4 |
| anti-Spy0167 P427L-W535F, | 1/5 | 0/4 |
| alum adjuvant | | |

TABLE 23
wild-type Spy0167 (5 μg/mouse)

| Sera | serum dilution | dead/treated |
|---|---|---|
| anti-Spy0167 P427L-W535F, | 1/5 | 0/4 |
| alum adjuvant | | |
| negative control (alum alone) | 1/5 | 4/4 |

### Example 22

### Immunization with Spy0167 P427L-W535F Protects Mice Against Intravenous Injection of Wild-Type Spy0167

Mice were immunized intraperitoneally three times (day 0, day 21, and day 35) with either wild-type Spy0167 or with the Spy0167 mutant P427L-W535F using alum as an adjuvant (20 μg protein in 2 mg/ml aluminium hydroxide). Mice immunized with adjuvant alone were used as a negative control. On day 55 mice were injected intravenously with different concentrations of a solution of wild-type Spy0167 in PBS, 2 mM DTT and monitored for at least 72 hours. The results are shown in Table 24.

TABLE 24

| | Dose of wild-type tagless Spy0167 injected into mouse tail vein | | | |
|---|---|---|---|---|
| | 2.5 μg/mouse | 5 μg/mouse | 10 μg/mouse | 20 μg/mouse |
| | survival (no. of | survival (no. of | survival (no. of | survival (no. of |
| | mice treated) | mice treated) | mice treated) | mice treated) |
| adjuvant (alum) | 100% (4) | 0% (12) | not tested | not tested |
| wild-type | not tested | 100% (8) | 100% (4) | 100% (4) |
| Spy0167 tagless | | | | |
| Spy0167 P427L- | not tested | 100% (8) | 100% (4) | 100% (4) |
| W535F tagless | | | | |

Five μg/mouse of wild-type Spy0167 is lethal for mice immunized with adjuvant alone; these mice died within a few minutes after Spy0167 injection. However, even 20 μg/mouse of the same wild-type Spy0167 preparation did not kill mice immunized with either wild-type Spy0167 or with the P427L-W535F Spy0167 mutant.

### Example 23

### Protection Against Intranasal Challenge with GAS M1 Strain by Spy0167 Mutant P427L-W535F

Thirty mice were immunized intraperitoneally with the Spy0167 mutant P427L-W535F, with either Alum or MF59 as adjuvants, and challenged intranasally with a GAS M1 strain. The results are shown in FIG. 30. Seventy-seven percent of the mice immunized with the Spy0167 mutant P427L-W535F and Alum were protected against intranasal challenge with a GAS M1 strain, as compared with 3% of the negative control mice (immunized with adjuvant only). Ninety percent of the mice immunized with the Spy0167 mutant P427L-W535F and MF59 were protected against intranasal challenge with a GAS M1 strain, as compared with 10% of the negative control mice (immunized with adjuvant only). These protection levels are comparable with those obtained by immunizing mice with wild-type Spy0167.

### Example 24

### In Vivo Protection Studies of Mice Immunized with GAS Antigens

This example provides the results of immunogenicity/protection tests carried out with various combinations of GAS antigens and/or GAS-specific polysaccharide conjugated with CRM197 (GC) following challenge with GAS strains of different M types. GAS proteins and GC were formulated either with Freund's adjuvant, aluminium hydroxide, or MF59. Protein antigen doses were 20 μg when used alone; protein combination formulations contained 20 μg each of wild-type Spy0269 (SEQ ID NO:177) and Spy0416 D151A/S617A (SEQ ID NO:198) and 10 μg of Spy0617 P427L/W535F (SEQ ID NO:125). GC doses are indicated in the tables.

The immunization schedule involved three doses at days 0, 21, and 35. Bleedings were done before first immunization and two weeks after the third immunization. Negative control groups were immunized with adjuvant only. Positive control groups were immunized with M protein homologous to the challenge strain.

Two weeks after the third immunization, mice were infected with lethal doses ranging from 2.5×10⁶ to 2.5×10⁸ (intranasal infection) or 20 to 2.5×10⁶ (intraperitoneal infection), depending on the challenge strain used. Survival rates were determined and are reported in Tables 25 and 26. The p-value was calculated with Fisher's test.

Immunogenicity was tested by ELISA.

Protection by Single Antigens and their Combination in Freund's Adjuvant Against Intranasal Infection with M1, M12, and M23

Table 25 reports the results of experiments in which mice were immunized with Spy0269 (SEQ ID NO:177), Spy0416 D151A/S617A (SEQ ID NO:198), or Spy0617 P427L/W535F (SEQ ID NO:125), or a combination of these antigens (“combo”) formulated with Freund's adjuvant and then challenged intranasally with M1, M12 and M23 strains. The results indicate that:
a. Spy0269 confers statistically significant protection against M1, M12 and M23 infection;
b. Spy0416 D151A/S617A and Spy0617 P427L/W535F confer significant protection against intranasal infection with M1 serotype; and
c. the combination of Spy0269, Spy0416 D151A/S617A and Spy0617 P427L/W535F confers >40% protection against M1, M12 and M23 GAS serotypes.

TABLE 25

| | M1 3348 | | | M12 EM5 | | | M23 2071 | | |
|---|---|---|---|---|---|---|---|---|---|
| antigen | Live/Total | % surv | Pval | Live/Total | % surv | Pval | Live/Total | % surv | Pval |
| Spy0269 | 82/145 | 57 | 0.0001 | 73/165 | 44 | 0.0001 | 23/35 | 66 | 0.0001 |
| Spy0416 D151A/S617A | 114/168 | 68 | 0.0001 | 22/80 | 28 | 0.1855 | 27/60 | 45 | 0.1855 |
| Spy0167 P427L/W534F | 105/114 | 92 | 0.0001 | 23/80 | 29 | 0.4842 | 7/20 | 35 | 0.2733 |
| combo | 145/152 | 95 | 0.0001 | 33/80 | 41 | 0.1354 | 39/80 | 49 | 0.0002 |
| M protein | 176/184 | 96 | | 136/180 | 76 | | 78/79 | 99 | |
| negative | 67/241 | 28 | | 64/258 | 25 | | 33/134 | 25 | |

Protection by the Combination of GAS25, GAS40, and GAS57 Antigens Plus GC, Formulated with Alum Against Intraperitoneal Infection with M1

Table 26 reports the results of experiments in which mice were immunized with the combination of Spy0167 mutant P427L/W535F, wild-type Spy0269, and Spy0416 mutant D151A/S617A (“combo”) with or without GC formulated with Alum and then challenged intraperitoneally with M1. The results indicate that statistically significant protection was obtained both with the protein combination alone and with the protein combination plus GC. Thus, even in combination, the exceptional immunogenicity of these GAS antigens is maintained.

TABLE 26

| | M1 3348 | | |
|---|---|---|---|
| antigen | Live/Total | % Surv | Pval |
| combo | 45/92 | 49 | 0.0001 |
| GC | 82/168 | 49 | 0.0001 |
| combo + GC | 36/72 | 50 | 0.0001 |
| M protein | 51/58 | 88 | 0.0001 |
| negative | 36/252 | 14 | |

### Example 25

### Cell Binding Assay

Human (A549, HeLa, 293, Detroit, ME180) or monkey (LLCMK2) epithelial cell lines are non-enzymatically detached from their support using a cell dissociation solution (Sigma), harvested, and suspended in Dulbecco's modified Eagle medium (DMEM). Approximately 2×10⁵ cells are mixed with either medium alone or with different Spy0269 recombinant protein concentrations (μg/ml) in a total volume of 200 ml in 96-well plates with U-shaped bottoms. Incubation at 4° C. is carried out for 1 hour. After two washes with PBS, cells are incubated with Spy0269 antibodies or antiserum (e.g., for antiserum, 1:200 in PBS/BSA 1%) for 1 hour at 4° C. After two washes, the samples are incubated at 4° C. for 30 minutes with a secondary antibody (e.g., for a mouse Spy0269 antiserum, the secondary antibody can be a R-phycoerythrin-conjugated goat F(ab)₂ antibody specific for mouse immunoglobulin diluted 1:100 in PBS/BSA 1%). Binding reactions are analyzed by flow cytometry. The mean fluorescence intensity for each population is calculated.

### Example 26

### Opsonophagocytosis Assay

This example describes the opsonophagocytosis assay used in the Examples below. Briefly, bacteria (10-50 colony forming units, CFUs, 25 μl in PBS) are incubated with 225 μl of whole blood from rabbits immunized either with adjuvant alone or with the tested antigen(s). The samples are incubated 5 hr at 37° with end-over-end rotation. Following dilution, samples are plated on blood agar plates and the number of CFUs is estimated.

In this assay, background killing by sera from animals immunized with adjuvant alone ranges from 7-36%. Killing activity by antigens varies but is consistently positive (e.g., 72-97% for M1 antibodies, 47-64% for GC antibodies, and 76-85% for antibodies raised against the combination of wild-type Spy0269 (SEQ ID NO:177), Spy0167 double mutant P427L/W535F (SEQ ID NO:125), and Spy0416 double mutant D151A/S617A (SEQ ID NO:198).

### Example 27

### Whole Blood Bactericidal Assays Demonstrating that Anti-Glycoconjugate (GC) Antibodies Mediate Killing of S. pyogenes

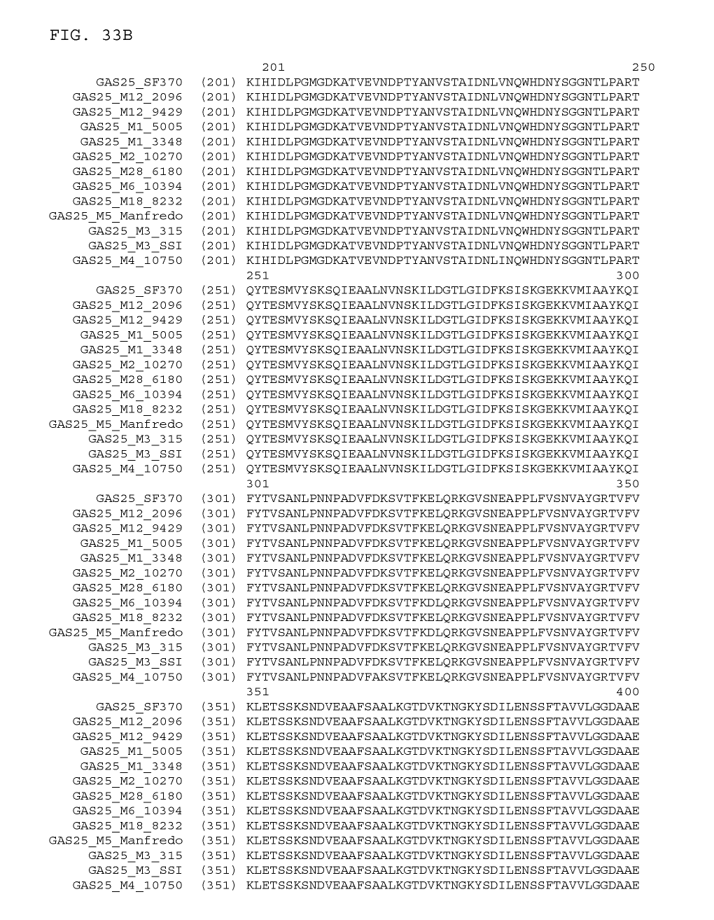

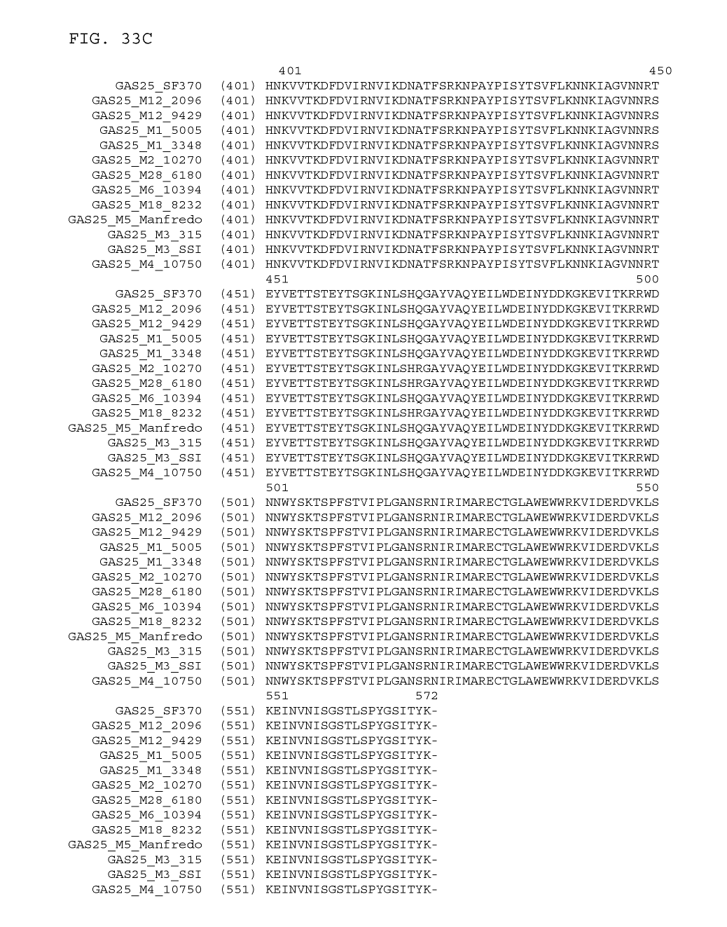

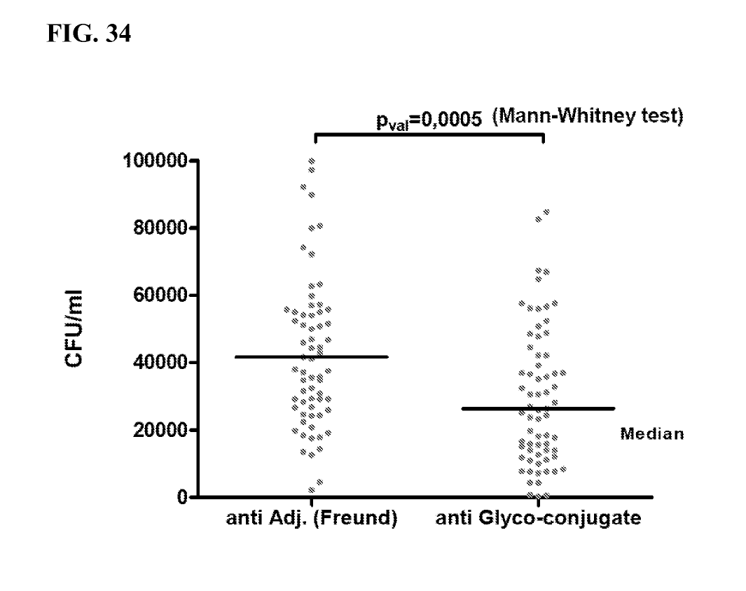

The assay described in Example 26 was carried out using whole blood obtained from rabbits immunized with 100 μg GC). The results, shown in FIG. 34, demonstrate that anti-GC antibodies mediate killing of S. pyogenes.

### Example 28

### Whole Blood Bactericidal Assays Demonstrating that the Combination of Anti-Glycoconjugate (GC) Antibodies and Antibodies Generated Against GAS Antigen Combinations Enhance Killing of S. pyogenes

The assay described in Example 26 was carried out with whole blood obtained from rabbits immunized with (a) Freund's adjuvant, (b) M1 protein, (c) a combination of wild-type Spy0269 (SEQ ID NO:177), Spy0167 double mutant P427L/W535F (SEQ ID NO:125), and Spy0416 double mutant D151A/S617A (SEQ ID NO:198) (100 μg each), (d) GC, and (e) the combination of wild-type Spy0269 (SEQ ID NO:177), Spy0167 double mutant P427L/W535F (SEQ ID NO:125), Spy0416 double mutant D151A/S617A (SEQ ID NO:198), and GC. The results are shown in FIG. 35.

It is desirable for a GAS vaccine to be bactericidal as well as immunogenic. These results demonstrate that, even in combination, these GAS antigens have bactericidal activity. The results also demonstrate a higher bactericidal effect of the combination of GAS antigens and GC antigen compared with that of either the GAS antigen combination or GC antigen alone.

### Example 29

### Experiments Demonstrating Lack of Cellular Toxicity of GAS Antigens

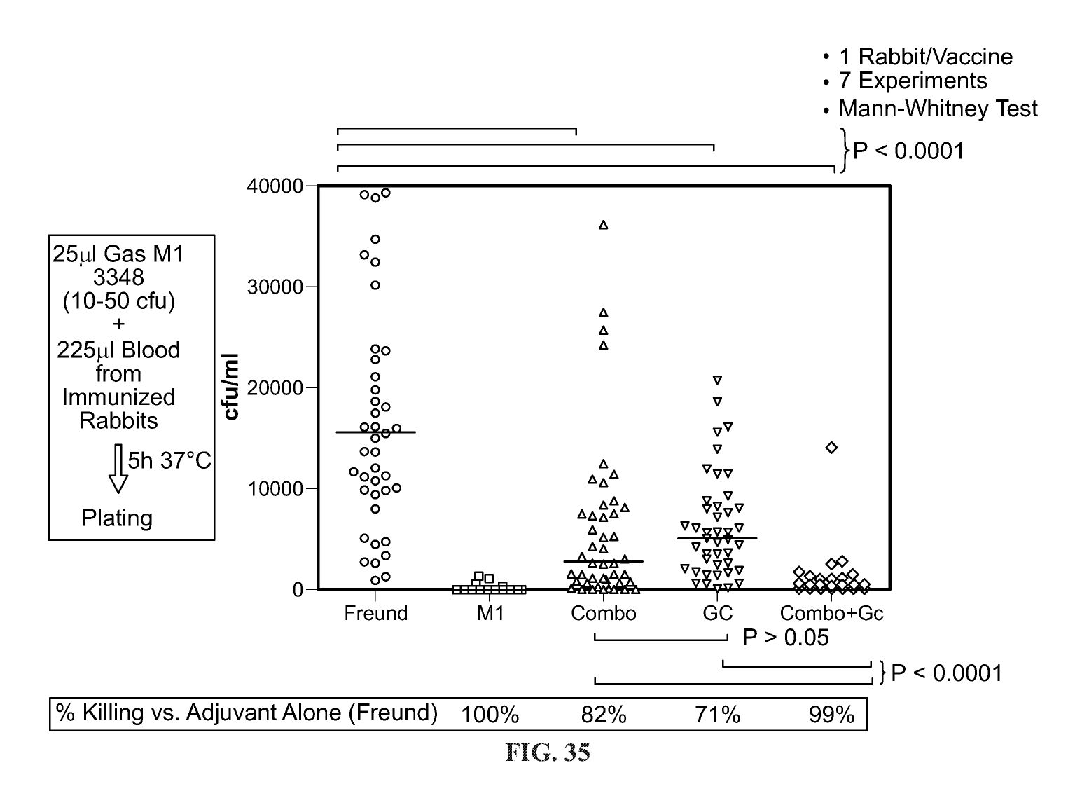

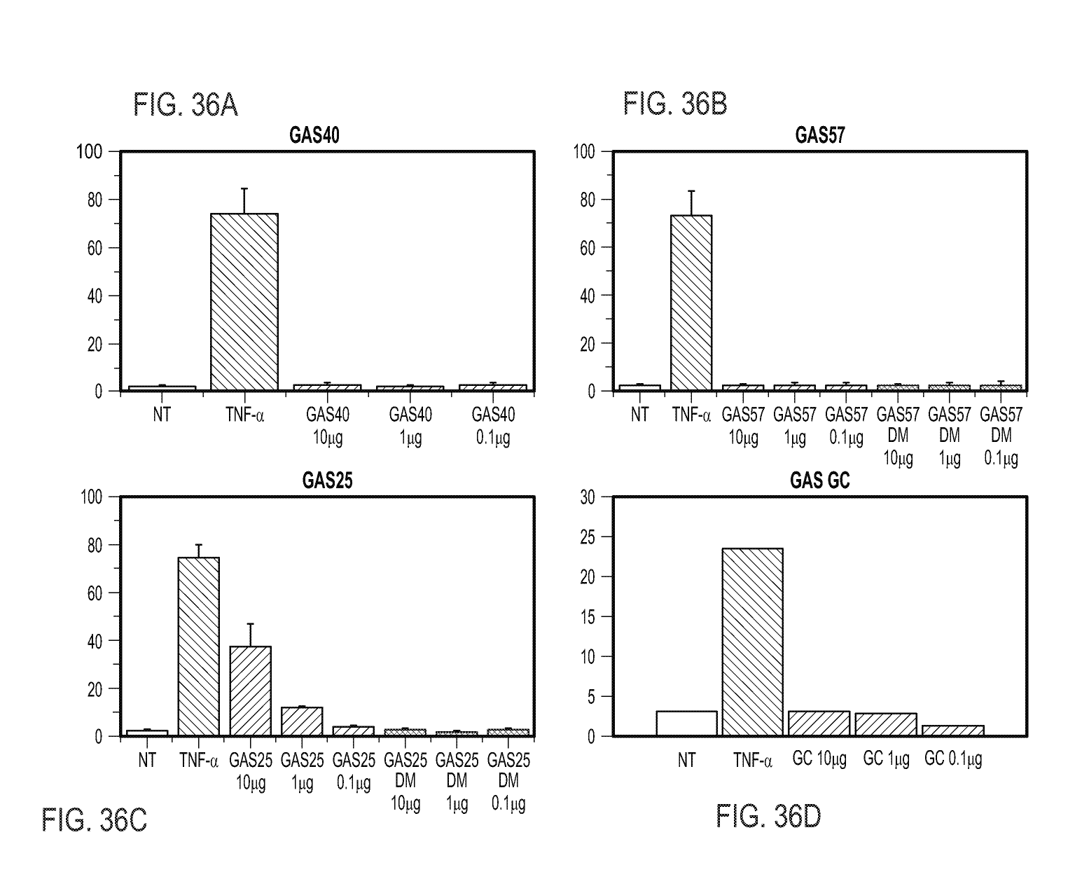

Endothelial cells human brain microvascular endothelial cells (HBMECs) were treated in vitro for 24 hours with various concentrations of recombinant GAS antigens in RPMI 1640 medium. Negative controls were not treated (“NT”), and cells treated with TNFα 1 μg/ml were used as positive controls Annexin V and propidium iodide staining were used to measure the percentage of apoptotic cells by flow cytometry. The results indicate no significant toxicity at the concentrations of wild-type Spy0269 (SEQ ID NO:177), Spy0167 double mutant P427L/W535F (SEQ ID NO:125), Spy0416 double mutant D151A/S617A (SEQ ID NO:198), and glycoconjugate (“GAS GC”) used in these Examples. See FIGS. 36A-D.

### Example 30

### Protein Antigen Conservation and Expression

The table below shows the average percent identity for each of Spy0269, Spy0416, and

Spy0167 among 57, 49, and 13 S. pyogenes strains, respectively.

| antigen | % identity (no. strains analyzed) | FACS positive |
|---|---|---|
| Spy0269 | 93% (57 strains) | 119/188 (63.3%) |
| Spy0416 | 95% (49 strains) | 98/174 (56.3%) |
| Spy0167 | 97% (13 strains) | 32/60 (53.3%) |

### Example 31

### ELISA Assays

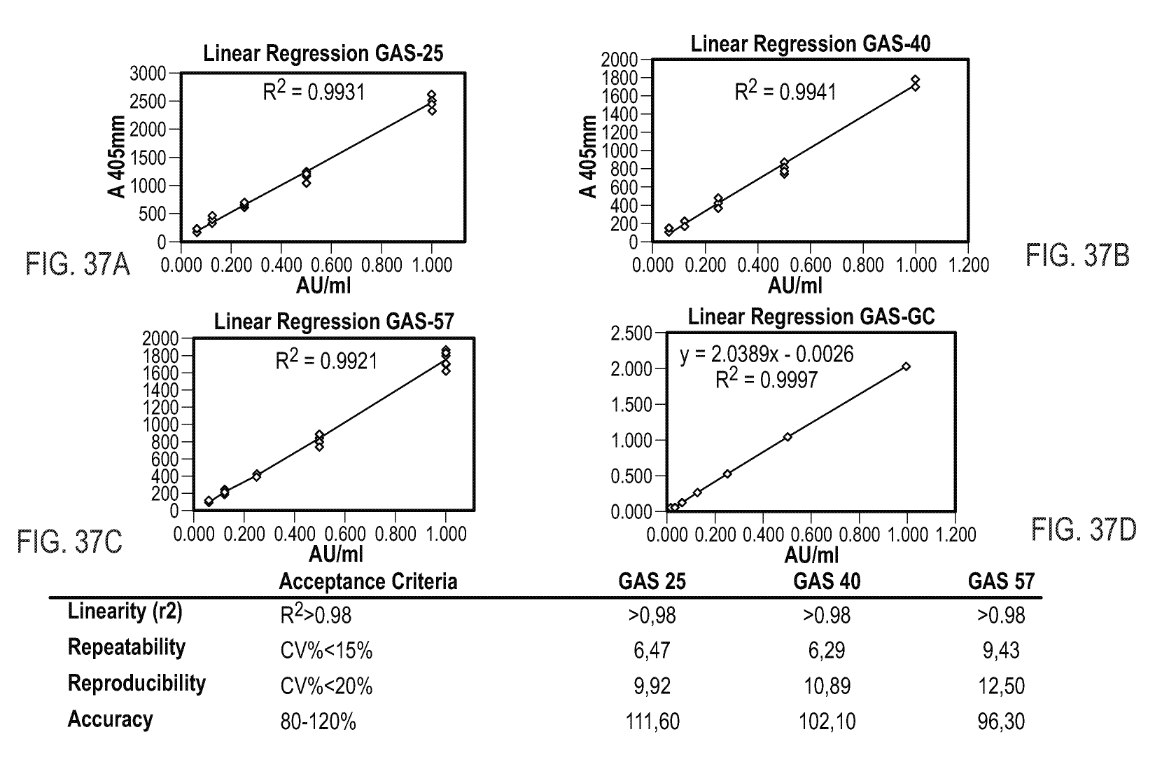

Briefly, plates are coated with antigen (0.1-0.3 μg/well) and blocked with 2% bovine serum albumin (BSA) in phosphate-buffered saline (PBS). After incubation with two-fold serial dilutions of the tested sera, plates are washed with 2% bovine serum albumin (BSA) in phosphate-buffered saline (PBS), and 0.05% TWEEN20® and incubated with secondary antibody (anti-total IgG, 1:2000) conjugated with alkaline phosphatase. After incubation with the substrate p-nitrophenyl phosphate (pNPP, 3 μg/ml), absorbance is measured at 405 nm. Serum titers are calculated by interpolating ODs from a standard curve. This assay is linear and reproducible, as shown in FIGS. 37A-D.

### Example 32

### In Vivo Challenge Experiments

CD1 5-6 week old female mice were immunized intraperitoneally 3 times on days 0, 21 and 35 with various doses of GAS antigens adjuvanted with alum in PBS and challenged either intranasally (50-ml Todd Hewitt containing an LD90 bacterial dose) or intraperitoneally (200 μl Todd Hewitt containing an LD90 bacterial dose) with various strains of S. pyogenes. Results are shown in Tables 27 and 28. In Tables 27 and 28, “40,” “25,” and “57,” respectively, are wild-type Spy0269 (SEQ ID NO:177), Spy0167 double mutant P427L/W535F (SEQ ID NO:125), and Spy0416 double mutant D151A/S617A (SEQ ID NO:198).

TABLE 27
Protection conferred by GAS antigens and combinations of GAS antigens in an
intraperitoneal challenge model.

| | | Challenge serotypes/strains | | | | | |
|---|---|---|---|---|---|---|---|
| | | M1 3348 | | M23 2071 | | M6 S43 | |
| | | % survival | | % survival | | % survival | |
| adjuvant | antigen | (no. mice) | P | (no. mice) | P | (no. mice) | P |
| | GC | 47 (176) | <0.001 | 30 (80) | <0.001 | 33 (104) | <0.001 |
| | 25 + 40 + 57 | 79 (156) | <0.001 | 36 (64) | <0.001 | 36 (88) | <0.001 |
| | 25 + 40 + 57 + GC | 74 (80) | <0.001 | 48 (64) | <0.001 | 48 (88) | <0.001 |
| alum | adjuvant alone | 14 (>100) | | 10 (>100) | | 11 (>100) | |

TABLE 28
Protection conferred by GAS antigens and combinations of GAS antigens in an
intranasal challenge model.

| | | challenge serotypes/strains | | | | | | | |
|---|---|---|---|---|---|---|---|---|---|
| | | M1 3348 | | M12 EM5 | | M23 2071 | | M6 S43 | |
| | | % | | % | | % | | % | |
| | | survival | | survival | | survival | | survival | |
| | GAS | (no. | | (no. | | (no. | | (no. | |
| adjuvant | antigen | mice) | P | mice) | P | mice) | P | mice) | P |
| Freund's | 25 | 95 (100) | <0.001 | 38 (50) | | 30 (60) | | | |
| | 40 | 45 (82) | <0.001 | 42 (150) | <0.001 | 66 (35) | <0.001 | | |
| | 57 | 72 (143) | <0.001 | 33 (189) | <0.05 | 39 (80) | | | |
| | 25 + 40 + 57 | 96 (56) | <0.001 | 40 (100) | <0.05 | 49 (80) | <0.001 | | |
| | (adjuvant | 23 (>100) | | 21 (>100) | | 21 (>100) | | | |
| | alone) | | | | | | | | |
| alum | 25 | 88 (48) | <0.001 | | | | | | |
| | 40 | 27 (130) | <0.05 | | | | | | |
| | 57 | 38 (157) | <0.001 | | | | | | |
| | 40 + 57 | 67 (46) | <0.001 | | | | | | |
| | 25 + 40 + 57 | 87 (280) | <0.001 | 33 (128) | <0.001 | 54 (48) | <0.001 | 53 (64) | <0.001 |
| | (adjuvant | 15 (>100) | | 9 (128) | | 10 (48) | | 22 (64) | |
| | alone) | | | | | | | | |

### Example 33

### Inclusion of Alum Provides Protection Against Strain M1 3348

This example demonstrates that inclusion of Alum in both Spy0167 and in combination formulations provides protection against S. pyogenes strain M1 3348.

CD1 5-6 week female mice were immunized with Spy0167 (GAS25) 10 μg or with a combination of Spy0167 (10 μg) together with Spy0269 (GAS40, 20 μg) and Spy0416 (GAS57, 20 μg) (“combination”), with or without alum. Animals were immunized intraperitoneally with 3 doses at days 0, 21 and 35. Intranasal challenge with M1 3348 was carried out essentially as described in Example 4. The results are shown in Table 29.

TABLE 29
Effect of including alum on survival after intranasal
challenge with M1 3348.

| | | % survival after M1 3348 |
|---|---|---|
| antigen | adjuvant | challenge (no. animals) |
| Spy0167 | alum | 84 (32) |
| Spy0167 | — | 29 (32) |
| — | alum | 14 (31) |
| combination | alum | 66 (32) |
| combination | | 23 (32) |
| — | alum | 14 (29) |

### Example 34

### Stability of GAS Antigen Formulations

Stability and in vivo potency of a combination GAS antigen formulation containing 100 μg/ml Spy0269 (1 mg/ml solution in PBS), 100 μg Spy0416 double mutant D151A/S617A (1 mg/ml solution in PBS), 50 μg Spy0167 double mutant P427L/W535F (1 mg/ml solution in PBS), 2 mg/ml aluminum hydroxide, 10 mM histidine buffer (pH 7.0), 9 g/l sodium chloride, with a pH of 7.0 +/− 0.3, with an osmolality of 300 +/− 20 mOsm/kg was tested by SDS-PAGE analysis for antigen integrity. The formulation is stable up to 1 year at 4° C. Antigen stability was evaluated by incubating at 2-8° C. over a one year period. All three protein components appeared quite stable after one year as assessed by SDS-PAGE. The protein antigens remain adsorbed to alum (>97.5%) for at least 36 weeks 2-8° C.

### Example 35

### Effect of Spy0416 and Spy0167 Antibodies

This example demonstrates that antibodies to Spy0416 and Spy0167 block toxic activity.

Spy0416:

Spy0416 was pre-incubated with pools of mouse specific sera or with a human serum with high ELISA titres to Spy0416. The mix was then incubated with IL-8 (10 μg/ml), and then tested for the presence of uncleaved IL-8 using an antibody which is specific for the cytokine but which is unable to recognize the cleaved inactive form. The results are expressed as percentage of uncleaved IL-8 calculated as follows:

$$\frac{[ IL \text{-} 8 in the reaction mix ]}{[ IL \text{-} 8 in the control mix ] ,} \times 100$$

where “control mix” is the reaction mix without the enzyme at time point 0.

Spy0167:

Wildtype Spy0167 was pre-incubated with a pool of sera from mice immunized either with 20 μg of Spy0167P427L/W535F or with adjuvant alone, and with human sera from responders and non responders. The mix was added to a sheep blood cell suspension and the OD540 nm decrease of the reaction supernatant was determined. Inhibition titer is expressed as the serum dilution required to reduce Spy0167-induced hemolysis by 50%.

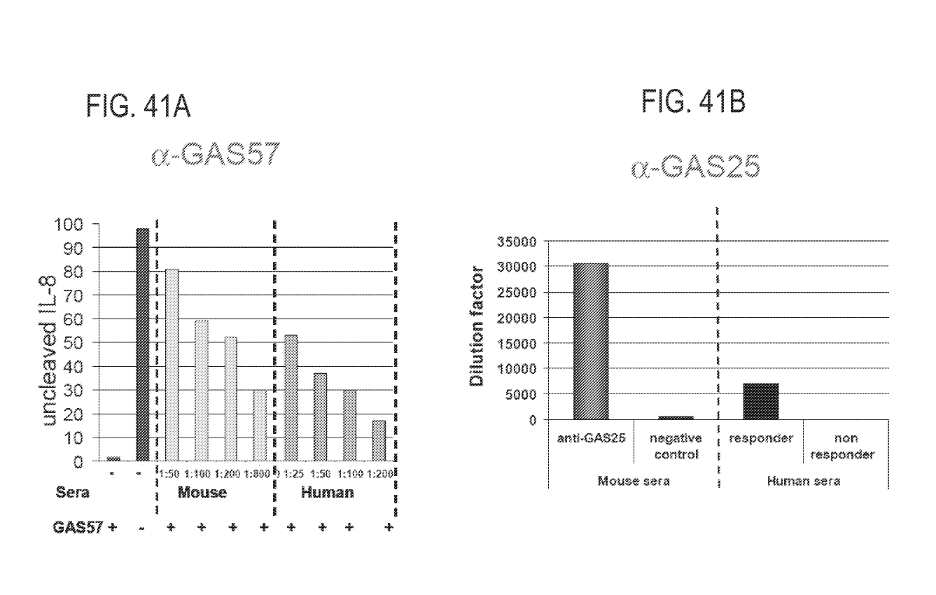

The results are shown in FIGS. 41A-B.

### Example 36

### Dose-Range Experiments

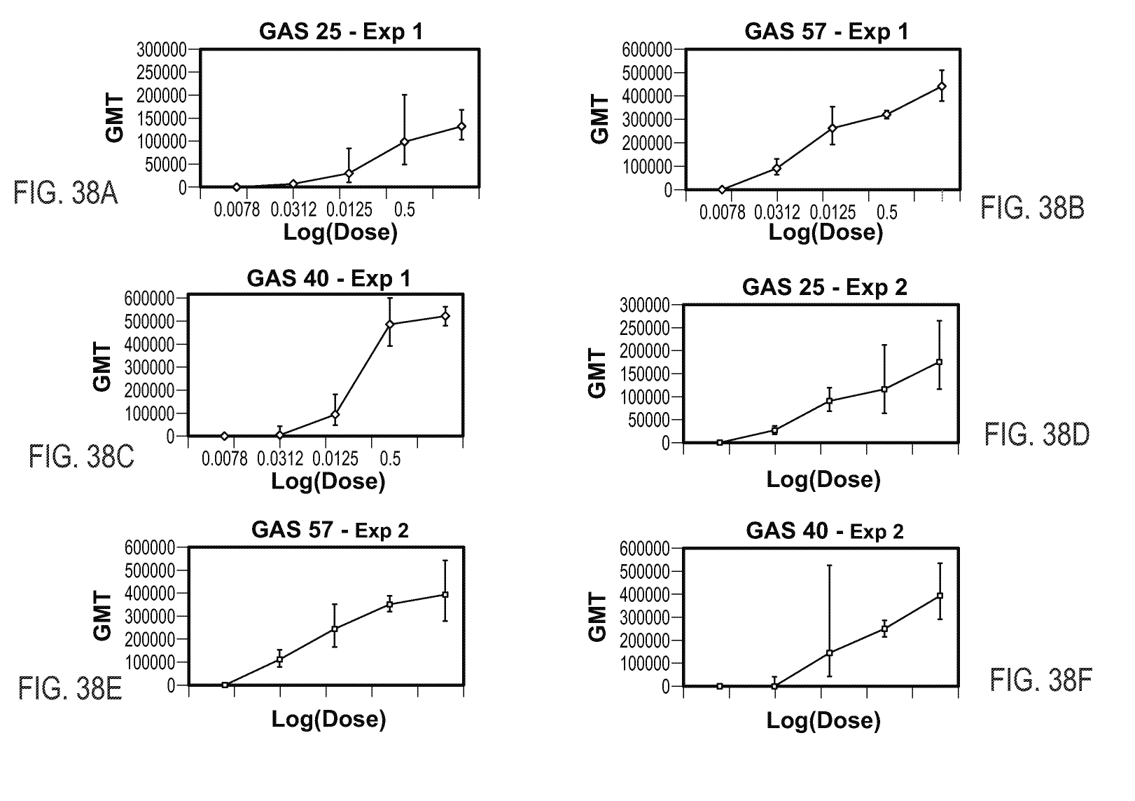

Five-week old female CD 1 mice were immunized with varying doses of wild-type Spy0269 (SEQ ID NO:177), Spy0416 mutant D151A/S617A (SEQ ID NO:198), and Spy0167 mutant P427L/W535F (SEQ ID NO:125) on days 0, 21, and 35. Dose-dependent IgG responses in the mice were measured by ELISA as described in Example 31. The results are shown in FIGS. 38A-C.

Mice were immunized with individual GAS protein antigens at various concentrations and challenged intranasally with S. pyogenes M1. The results are shown in Table 30.

TABLE 30

| protein | dose (μg) | mice | dead | % survival |
|---|---|---|---|---|
| GAS40 | 2 | 32 | 20 | 38 |
| | 20 | 32 | 18 | 44 |
| Spy0416 mutant D151A/S617A | 2 | 32 | 16 | 53 |
| | 20 | 32 | 19 | 43 |
| Spy0167 mutant | 2 | 32 | 24 | 75 |
| P427L/W535F (SEQ ID NO: 125) | 0.5 | 32 | 28 | 87 |
| | 0.125 | 32 | 20 | 62 |
| (PBS) | | 32 | 27 | 16 |

As shown in Table 30, there is no clear dose-dependent protection, indicating that a variety of concentrations of these antigens are useful for achieving protection against S. pyogenes challenge.

Mice were immunized with the combination of 20 μg wild-type Spy0269 (SEQ ID NO:177), 10 μg Spy0167 double mutant P427L/W535F (SEQ ID NO:125), and 20 μg Spy0416 double mutant D151A/S617A (SEQ ID NO:198) at various concentrations and challenged intranasally with S. pyogenes M1. The results are shown in Table 31.

TABLE 31

| protein | dose (μg) | mice | dead | % survival |
|---|---|---|---|---|
| combination | 20 + 20 + 10 | 16 | 11 | 31 |
| | 20 + 20 + 2 | 16 | 10 | 38 |
| | 20 + 2 + 10 | 16 | 6 | 63 |
| | 20 + 2 + 2 | 16 | 7 | 50 |
| | 2 + 20 + 10 | 16 | 2 | 88 |
| | 2 + 20 + 2 | 16 | 9 | 44 |
| | 2 + 2 + 10 | 16 | 16 | 0 |
| | 2 + 2 + 2 | 16 | 14 | 13 |
| none | 0 + 0 + 0 | 16 | 15 | 6 |

As with the single antigen dose experiments described above, there is no clear dose-dependent protection, indicating that, even in combination, a variety of concentrations of these antigens are useful for achieving protection against S. pyogenes challenge.

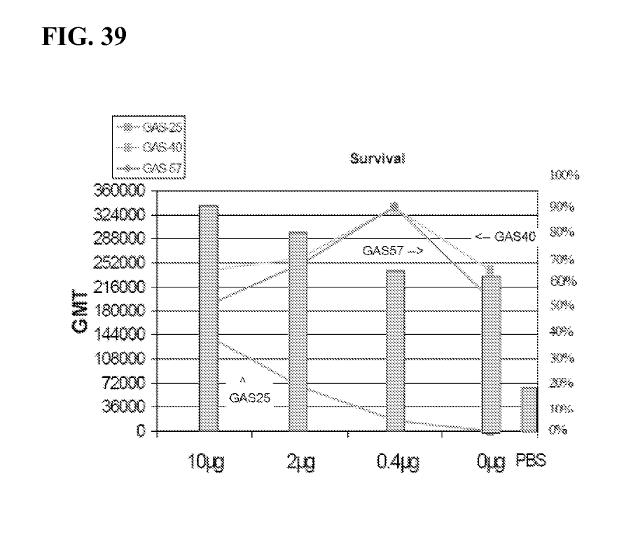

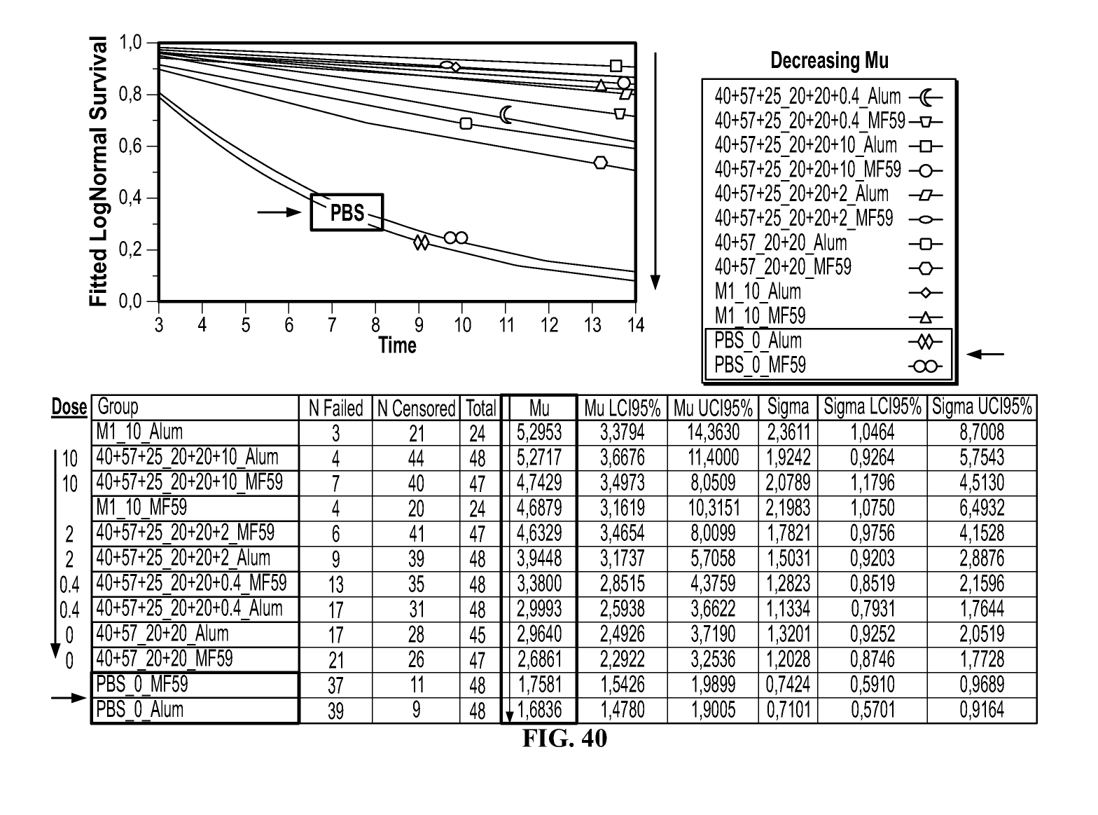

The results are summarized in FIG. 39. FIG. 40 shows an analysis of a LogNormal model adopted as a first approximation of mean survival time (MST; Mu).

1. A composition comprising:
(a) a purified Spy0269 protein comprising the amino acid sequence SEQ ID NO:177;
(b) a purified mutant Spy0167 protein comprising the amino acid sequence SEQ ID NO:125; and
(c) a purified mutant Spy0416 protein comprising the amino acid sequence SEQ ID NO:149.

2. The composition of claim 1 wherein at least one of the purified Spy0269, mutant Spy0167, and mutant Spy0416 proteins is produced recombinantly.

3. The composition of claim 1 further comprising a group A polysaccharide of formula 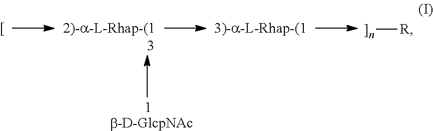
wherein R is a terminal reducing L-rhamnose or D-GlcpNAc and n is a number from about 3 to about 30.

4. The composition of claim 1 further comprising an adjuvant.

5. The composition of claim 4 wherein the adjuvant is alum.

6. The composition of claim 1 further comprising a pharmaceutically acceptable carrier.

7. The composition of claim 1 further comprising a group A polysaccharide, alum, and a pharmaceutically acceptable carrier.

8. The composition of claim 1 further comprising a polypeptide antigen which is useful in a pediatric vaccine.

9. The composition of claim 8 wherein the polypeptide antigen is selected from the group consisting of N. meningitidis, S. pneumoniae, Bordetella pertussis, Moraxella catarrhalis, Clostridium tetani, Chorynebacterium diphtheriae, respiratory syncytial virus, polio virus, measles virus, mumps virus, rubella virus, and rotavirus polypeptide antigens.

10. The composition of claim 1 further comprising a polypeptide antigen which is useful in a vaccine for elderly or immunocompromised individuals.

11. The composition of claim 10 wherein the polypeptide antigen is selected from the group consisting of Enterococcus faecalis, Staphylococcus aureus, Staphylococcus epidermidis, Pseudomonas aeruginosa, Legionella pneumophila, Listeria monocytogenes, influenza virus, and parainfluenza virus polypeptide antigens.

12. A method of treating or reducing risk of infection by Streptococcus pyogenes, administering to an individual in need thereof an effective amount of a composition comprising:
(a) a purified Spy0269 protein comprising the amino acid sequence SEQ ID NO:177;
(b) a purified mutant Spy0167 protein comprising the amino acid sequence SEQ ID NO:125; and
(c) a purified mutant Spy0416 protein comprising the amino acid sequence SEQ ID NO:149.

13. The method of claim 12 wherein the composition further comprises one or both of:
(d) a group A polysaccharide of formula 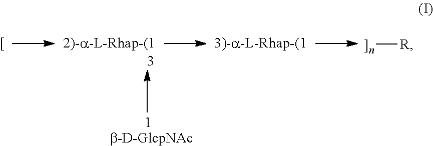
wherein R is a terminal reducing L-rhamnose or D-GlcpNAc and n is a number from about 3 to about 30; and
(e) an adjuvant.

14. A method of making a vaccine for treating Streptococcus pyogenes infection, comprising combining:
(a) a purified Spy0269 protein comprising the amino acid sequence SEQ ID NO:177;
(b) a purified mutant Spy0167 protein comprising the amino acid sequence SEQ ID NO:125;
(c) a purified mutant Spy0416 protein comprising the amino acid sequence SEQ ID NO:149; and
(d) a pharmaceutically acceptable carrier.

15. The method of claim 14 wherein at least one of the purified Spy0269, mutant Spy0167, and mutant Spy0416 proteins is produced recombinantly.

16. The method of claim 14 further comprising combining one or both of:
(e) a group A polysaccharide of formula 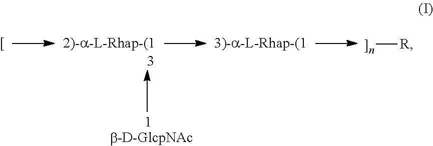
wherein R is a terminal reducing L-rhamnose or D-GlcpNAc and n is a number from about 3 to about 30; and
(f) an adjuvant.

17. The method of claim 16 wherein the adjuvant is alum.

18. An isolated host cell comprising one or more nucleic acid molecules which encode:
(a) a Spy0269 protein comprising the amino acid sequence SEQ ID NO:177;
(b) a mutant Spy0167 protein comprising the amino acid sequence SEQ ID NO:125; and
(c) a mutant Spy0416 protein comprising the amino acid sequence SEQ ID NO:149.

19. The isolated host cell of claim 18 which comprises:
(1) a first nucleic acid molecule which encodes the Spy0269 protein and a second nucleic acid molecule which encodes the mutant Spy0167 protein and the mutant Spy0416 protein; or
(2) a first nucleic acid molecule which encodes the Spy0269 protein and the mutant Spy0167 protein and a second nucleic acid molecule which encodes the mutant Spy0416 protein; or
(3) a first nucleic acid molecule which encodes the Spy0269 protein and a second nucleic acid molecule which encodes the mutant Spy0167 protein and the mutant Spy0416 protein.

20. The isolated host cell of claim 18 wherein each of the Spy0269 protein, the mutant Spy0167 protein and the mutant Spy0416 protein is encoded by a separate nucleic acid molecule.
